(19) United States
(12) Patent Application Publication
(10) Pub. No.: US 2005/0232922 A1
(43) Pub. Date: Oct. 20, 2005

(54) Oncofetal antigen specific T-lymphocyte mediated immune response: manipulation and uses of oncofetal antigen specific CD4, CD8 cytotoxic and suppressor T cells and interleukin-10

(76) Inventors: Joseph H. Coggin JR., Mobile, AL (US); James W. Rohrer, Wilmer, AL (US); Adel L. Barsoum, Mobile, AL (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 11/145,037

(22) Filed: Jun. 3, 2005

Related U.S. Application Data
(63) Continuation of application No. 10/294,524, filed on Nov. 14, 2002, now abandoned.
Application No. 10/294,524 is a continuation of application No. 09/173,912, filed on Oct. 16, 1998, now abandoned.
Application No. 09/173,912 is a continuation-in-part of application No. 08/835,069, filed on Apr. 4, 1997, now Pat. No. 6,335,174.
(60) Provisional application No. 60/014,903, filed on Apr. 5, 1996.

(51) Int. Cl. A61K 39/395

(52) U.S. Cl. 424/145.1

(57) ABSTRACT

Disclosed are methods for detecting cancer or determining the success of cancer therapy in an individual. These methods are based on analyzing the presence or frequency of cloned oncofetal antigen (OFA)- or immature laminin receptor protein (iLRP)-specific T lymphocyte subclasses obtained from the individual and which are stimulated with 44 kD OFA or iLRPA. A frequency of CD8 cytotoxic T cells relative to CD8 T suppressor cells indicates effectiveness of therapy, and a likelihood that protective immunity will develop. Also disclosed are kits for conducting these methods. Further disclosed are methods of rendering T suppressor lymphocytes cytotoxic, and methods of clonally expanding cytotoxic T lymphocytes in vivo.

### CROSS-REFERENCE TO RELATED APPLICATIONS

This application is a continuation of U.S. application Ser. No. 10/294,524, filed Nov. 14, 2002, which is a continuation of U.S. application Ser. No. 09/173,912, filed Oct. 16, 1998, abandoned, which is a continuation-in-part of U.S. application Ser. No. 08/835,069, filed Apr. 4, 1997, now U.S. Pat. No. 6,335,174, issued Jan. 1, 2002, and which claims priority to provisional application U.S. Ser. No. 60/014,903, filed Apr. 5, 1996.

### STATEMENT REGARDING FEDERALLY SPONSORED RESEARCH

This invention was produced in part using funds obtained through a grant from the National Institutes of Health. Consequently, the federal government has certain rights in this invention.

### BACKGROUND OF THE INVENTION

The present invention relates generally to the fields of immunology and protein chemistry. More specifically, the present invention relates to oncofetal antigen specific T-lymphocyte subclass mediated immune responses: manipulation and uses of oncofetal antigen specific CD4, CD8 cytotoxic and suppressor T cells and interleukin-10 for early cancer detection tests, for conventional therapy monitoring, and for immune-intervention through autologous T-cell therapy and anti-cancer vaccination.

Established tumors can grow and kill the host bearing such tumors even though lymphocytes obtained from that host animal can adoptively transfer tumor immunity to other syngeneic animals. Leffel, et al., Cancer Res. 37:4112, (1977); North, et al., J. Exp. Med. 145:275, (1977); Gershon, et al., Nature 213:674, (1967). Also, investigators have shown that a tumor-bearing animal can reject challenge with part of that tumor when inoculated with tumor cells at a different site on its body. Leffel, et al., supra; Vaage, J., Cancer Res. 31:1655, (1971). This phenomenon has been termed concomitant immunity. North, et al., supra; Gershon, et al., supra; Vaage, supra. Tumors can evade tumor-reactive lymphocyte-mediated destruction by inhibiting protective immune responses directly, by secretion of inhibitory cytokines, and indirectly, by activating inhibitory regulatory elements of the immune system . Vose, et al., Int. J. Cancer 245:579, (1979); Yu, et al., N. Engl. J. Med. 297:121, (1977); Zarling, et al., Cancer Immun Immunother. 7:243, (1980); Cone, et al., J. Clin. Invest. 43:2241, (1964); Berg et al., J. Immunol. 154:718, (1995); Bost, et al., J. Immunol. 154:718, (1995); Smith, et al., Am. J. Pathol. 145:18, (1994).

It has been suggested that rodents, like humans, challenged with carcinogens such as DNA-altering chemicals, radiation or oncogenic viruses respond as either “progressors”, which develop advanced lethal tumors and die or “regressors”, which fail to develop the fully malignant tumor cells giving rise to cancer. The “regressors” immunologically manage to control the tumor's growth or existence. These protective immune mechanisms at work in the regressors are widely believed to occur through cell-mediated responses mediated by a T-cell subclass, termed CD8 T-Cytotoxic or TC cells, and/or assisted by other antigen-specific T-lymphocyte subclasses including CD4 T-Helper 1 or TH-1 subclasses.

Approximately 60% of RFM mice develop lethal thymic lymphomas during a six-month period subsequent to fractionated, sublethal X-irradiation. Coggin, et al., Am. J. Pathol. 130:136, (1988); Rohrer, S. D., et al., J. Natl. Cancer Inst. 84:602, (1992). Systematic sampling of thymocytes of the irradiated mice during the first six months post-irradiation using intrathymic challenge assay into normal syngeneic mice revealed that when any OFA⁺ thymocytes were transferred to normal thymus, a high correlation of adoptive induction of T-cell lymphoma was observed, suggesting that oncogenic cells were induced in all irradiated recipients by six months. However, only approximately half of the irradiated donor mice developed lymphomas. The irradiated mice that survive the first 6 months never show any physical signs of tumor development.

It has been shown that mice which had been irradiated 11 months previously and appeared tumor-free, had developed clonable memory CD4 and CD8 effector T cells which were specific for a 44 kDa oncofetal antigen (OFA). Rohrer, J. W., et al., J. Immunol. 154:2266, (1995). It was also determined that age-matched, non-irradiated RFM mice yielded OFA-specific T cell clones; however the frequency of these T cell clones was significantly lower than the frequency in the long-term irradiation survivors, and the non-irradiated mice yielded no clones with high affinity anti-OFA T cell receptors. Immunobiology teaches that animals and humans which retain the capacity to respond to T or B-cell stimulating immunogens retain low affinity precursors and are able to respond to such non-self. Thus it is not surprising that such OFA-reactive memory T cells would be induced in the irradiated mice, since OFA⁺ thymus cells are detectable as early as 2 weeks after irradiation but are entirely absent from non-irradiated, normal RFM/UnCr mice Rohrer, S. D., supra; Payne, et al., J. Natl. Cancer Inst. 75:527, (1985).

However, even with these memory effector T cells that are tumor-reactive, challenge of previously irradiated mice yielded no increased resistance to RFM lymphoma cells. In fact, such previously irradiated mice showed significantly enhanced tumor growth kinetics compared to non-irradiated, age-matched controls that were challenged with the same tumor cells. Rohrer, J. W., supra. This is likely because the previously irradiated, long-term survivor mice had not only effector T cells, but also CD8⁺ non-cytotoxic T cells that did not secrete interferon-γ. These non-cytotoxic CD8 T cells must secrete some factor(s) which inhibits the cytotoxic activity of anti-OFA cytotoxic T cell clones but does not inhibit T_{C} clone cell proliferation. Rohrer, J. W., supra.

All modern summaries of tumor immunobiology from other laboratories attempting to characterize a host's immune response to emerging antigenic cancers [e.g., Renie and Rusting, Sci. Amer. September: 57-59 (1996); Cox, Intern. J. Rad Biol. 65:57-64 (1994); Levy and Bost, Critical Reviews in Immunology 16:31-57 (1996); Chang and Shu, Critical Reviews in Oncology/Hematology 22:213-228 (1996); Kavanaugh and Carbone, Hematology/Oncology Clinics of North America: 4:927-951 (1996)] focus on the means by which the primary tumors and metastases “escape” the host's various humoral and cellular-mediated immune responses directed against the tumor. The focus has been instead on unshared, individual tumor specific transplantation antigen (TSTA). Rarely is a shared, host-cell encoded, tumor associated transplantation antigen (TATA) mentioned as the target for the specificity of these immune response. The 44 kD oncofetal antigen (44 kD OFA) is an antigen which is normally expressed in embryonic and fetal tissue as phase-specific, developmentally regulated, embryonic antigen. This OFA is distributed widely on all tumors of rodents and humans as a tumor-specific, but not a tumor subclass-specific, antigen or immugen. See, e.g., Coggin, et al. J. Natl. Cancer Inst., (in press) (1996); Rohrer, S., et al, J. Natl. Can. Inst., 84:602-609 (1992); Rohrer, J. W., et al, J. Immunol. 152:754-764 (1994); Rohrer, J., et al, J. Immunol., 154L 2266-2280 (1995); Rohrer and Coggin, J. Immunol., 155:5719-5727 (1995); Henderson and Finn, Advances in Immunology 62:217-256 (1996); Coggin, Shared Cross-Protective OFAs on Chemically Induced Rodent Sarcomas. Immunology Today. 10(3):76-78 (1989); Coggin, Molecular Biotherapy 1(4):223-228 (1989); Barsoum and Coggin, Journal of Biological Response Modifiers. 8:579-592 (1989); Barsoum and Coggin, Inter. J. Cancer 48:248-252 (1991); Barsoum and Coggin, Int. J. Biochem. 24:483-489 (1993); Coggin, et al., Archives of Otolaryngology-Head and Neck Surgery 119:1257-1266 (1993); Rashid, et al, J. Nat'l Cancer Inst 86:515-526 (1994); Payne and Coggin, J. Nat'l Cancer Inst. 75(3):115-132 (1985)].

The prior art is deficient in effective means for screening individuals for tumor marker expression, particularly during early stage carcinoma and/or leukemia or lymphoma development. In addition, the prior art is deficient in effective means for monitoring a patient's immune response during cancer treatment or therapy of the cancer.

### SUMMARY OF THE INVENTION

The present invention fulfills these longstanding needs and desires in the art. A first embodiment of Applicants' invention is directed to a qualitative or semi-quantitative method for screening an individual for cancer. The semi-quantitative method involves the steps of obtaining a sample of T-lymphocytes from an individual; cloning the lymphocytes, wherein cloned lymphocytes contain memory CD4 helper cell subclasses, CD8 cytotoxic T-lymphocyte subclasses and CD8 non-cytotoxic T-suppressor lymphocyte subclasses; contacting the cloned lymphocytes with a composition containing oncofetal antigen (OFA) (which is defined herein as 44 kDa OFA glycoprotein, the 37 kDa protein portion thereof, or an antigenically active fragment) or immature laminin receptor protein (iLRP), thereby stimulating OFA- or iLRP-specific T-cells comprising memory CD4 helper cells, CD8 cytotoxic T lymphocytes and CD8 non-cytotoxic T-suppressor lymphocytes; and determining a frequency of each of the T lymphocyte subclasses relative to each other as an indication of cancer. The qualitative method involves a different sequence of these steps. Once the T-lymphocyte sample is obtained, the composition containing OFA or iLRP is added, thereby stimulating OFA specific T-cells including memory CD4 helper cells, CD8 cytotoxic T lymphocytes and CD8 non-cytotoxic T-suppressor lymphocytes. The stimulated T-lymphocytes are then cloned and the presence of the various T cell subclasses, e.g., presence of cytotoxic T lymphocytes relative to said non-cytotoxic T-suppressor lymphocytes, is determined as an indication of cancer.

A related aspect of the present invention is directed to a method of monitoring cancer therapy, which like the aforementioned method, may be qualitative or semi-quantitative. The method entails the steps of: obtaining a sample of T-lymphocytes from a cancer patient undergoing therapy; cloning the lymphocytes, wherein cloned lymphocytes contain T cell subclasses including OFA- or iLRP-specific memory CD4 helper cells, CD8 cytotoxic T lymphocytes and CD8 non-cytotoxic T-suppressor lymphocytes; contacting the lymphocytes with a composition containing OFA or iLRP, preferably in purified form, thereby stimulating OFA- or iLRP-specific T-cell subclasses comprising memory CD4 helper cells, CD8 cytotoxic T lymphocytes and CD8 non-cytotoxic T-suppressor lymphocytes; and determining the presence or frequency of each of said T lymphocyte subclasses relative to each other as an indication of efficacy of the therapy. If a rapid, qualitative test is desired, the steps are performed in the same sequence as above, i.e., stimulating followed by cloning, whereas the semi-quantitative test based on frequency involves stimulating followed by cloning. A high frequency of CD8 cytotoxic cells relative to CD8 T suppressor cells, therapy is effective and the development of protective immunity is likely. Preferred sources of T-lymphocytes for practicing these methods include peripheral blood lymphocytes or in the case of therapy monitoring in a cancer patient, tumor infiltrating lymphocytes at a residual tumor site.

Another related aspect of the present invention is directed to a method of stimulating T-lymphocyte subclasses comprising memory CD4 helper cells, CD8 T_{C} cytotoxic lymphocytes and CD8 non-cytotoxic T-suppressor lymphocytes. A sample of T-lymphocytes is obtained from an individual, and the T-lymphocytes are contacted (e.g., cultured in the presence of) OFA or iLRP, thereby stimulating T cell subclasses including memory CD4 helper cells, CD8 Tc cytotoxic lymphocytes and CD8 non-cytotoxic T-suppressor lymphocytes.

Another related aspect of the present invention is directed to a kit useful in a method for detecting cancer or monitoring cancer therapy. The kit contains at least one cytokine which is interleukin-2 and/or interleukin-6, gamma-interferon, a T-lymphocyte growth medium, autologous antigen processing cells, OFA or iLRP, at least one reagent for measuring T cell DNA stimulation, and CD4, CD8 and interleukin-10 phenotyping reagents.

Another aspect of the present invention is directed to a method for rendering T-suppressor cells cytotoxic, comprising administering to an individual an agent, preferably an anti-IL-10 antibody, that selectively kills the T suppressor cells or otherwise inhibits or neutralizes IL-10 production by T suppressor cells.

A further aspect of Applicants' invention is directed to a method of distinguishing CD8 cytotoxic T-lymphocytes from CD8 T suppressor in a fluid or tissue sample. A detectably labeled anti-gamma interferon antibody is added to the sample under conditions that allow antibody binding to occur, wherein binding of said antibody indicates presence of CD8 cytotoxic T-lymphocytes.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention are attained and can be understood in detail, more particular descriptions of the invention may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows that the culture supernatants from non-cytotoxic, anti-OFA, CD8 T cell clones inhibit interferon-γ secretion by anti-OFA CD4 and CD8 T cell clones. The data are presented as mean IFN-γ concentration (pg/ml)±SEM. Experiments were repeated 3 times. FIG. 1A shows the effect on IFN-γ secretion by anti-OFA CD4 T cell clone 7 after preincubation for 24 hours with various amounts of culture supernatant collected from non-cytotoxic T cell clones 9, 10, and 11 or from T_{C} clone 4 one week after restimulation of the CD8⁺ clones with irradiated RFM 5T lymphoma cells+irradiated RFM T cell-depleted spleen cells+recombinant murine IL-2. FIG. 1B shows the effect on secretion of IFN-γ by anti-OFA CD8 T_{C} cell clone 1 after preincubation with various amounts of culture supernatant from non-cytotoxic T cell clones 9, 10, and 11 or from T_{C} clone 4 collected one week after restimulation of those CD8⁺ clones as described in FIG. 1A above.

FIG. 2 shows the inhibitory activity of culture supernatants from non-cytotoxic CD8, anti-OFA T cell clones for IFN-γ secretion is not antigen-specific. The data are presented as mean IFN-γ concentration (pg/ml)±SEM. Experiments were repeated 3 times. FIG. 2A shows the effect on IFN-γ secretion by OFA-specific CD4 RFM T cell clone 7 after preincubation with various amounts of culture supernatant from non-cytotoxic OFA-specific CD8 T cell clones, 9, 10, and 11 or from T_{C} clone 4 collected 1 week after restimulation of those clones with irradiated RFM 5T lymphoma cells+irradiated RFM T cell-depleted spleen cells+IL-2. FIG. 2B shows the effect on IFN-γ secretion by 5T TSTA-specific CD4 RFM T cell clone 1 after preincubation with various amounts of culture supernatant collected from RFM non-cytotoxic T cell clones, 9, 10, and 11 or from RFM T_{C} clone 4 one week after restimulation of the clones as described in FIG. 2A above.

FIG. 3 shows that the inhibitory activity of culture supernatants from non-cytotoxic CD8, anti-OFA T cell clones for IFN-γ secretion is not MHC-restricted. The data are presented as mean IFN-γ concentration (pg/ml)±SEM. Experiments were repeated 3 times. FIG. 3B shows the effect on IFN-γ secretion by RFM OFA-specific CD4 T cell clone 7 after preincubation with various amounts of culture supernatant collected from RFM non-cytotoxic CD8 T cell clones 9, 10, and 11 or from T_{C} clone 4 one week after restimulation of those clones with irradiated RFM 5T lymphoma cells+irradiated RFM T cell-depleted spleen cells+IL-2. FIG. 3B shows the effect on IFN-γ secretion by BALB/c OFA-specific CD4 T cell clone 5 after preincubation with various amounts of culture supernatant collected from RFM non-cytotoxic T cell clones 9, 10, and 11 or from T_{C} clone 4 one week after restimulation of those clones as described in FIG. 3A.

FIG. 4 shows the culture supernatants from RFM non-cytotoxic CD8, anti-OFA T cell clones, but not from RFM anti-OFA T_{C} clones contain IL-10. Culture supernatants collected 1 week after restimulation of anti-OFA non-cytotoxic CD8⁺ clones and anti-OFA CD8⁺ T_{C} clones with irradiated 5T lymphoma cells+irradiated, T cell-depleted, RFM spleen cells+IL-2 were assayed for IL-10 by a quantitative ELISA assay. Supernatants were collected three different times and the data are presented as mean IL-10 concentration (pg/ml)±SEM. The lowest amount of IL-10 detectable with this assay is 13 pg/ml.

FIG. 5 shows the RFM 5T lymphoma cells are not the source of the IL-10. One week after restimulation, the CD8 cytotoxic and non-cytotoxic clone cells and the 5T lymphoma cells were separated by a combination of negative and positive selection with anti-CD4 and anti-CD8 monoclonal antibodies localized to Petri plates. After separation the unselected or selected populations were cultured for 48 hours and their supernatants collected and assayed for IL-10 by a quantitative ELISA assay. Supernatants were collected three different times and the data are presented as mean IL-10 concentration (pg/ml)±SEM. The lowest amount of IL-10 detectable with this assay is 13 pg/ml.

FIG. 6 shows the macrophages in the T cell clone stimulation cultures are not the source of IL-10. One week after restimulation of cytotoxic and non-cytotoxic CD8 T cell clones with irradiated 5T cells, the cultures are harvested and the T cell clones separated from the lymphoma cells by negative selection with CD4 antibody localized to Petri plates and positive selection with CD8 plates. Unselected cultures and cultures depleted of macrophages by anti-CD11b antibody+anti-rat IgG+complement cytotoxicity were cultured separately and 48 hours later supernatants were collected and assayed for IL-10 by ELISA assay. The data are presented as mean IL-10 concentration (pg/ml)+SEM for 3 repeat experiments. The lowest amount of IL-10 detectable with this assay is 13 pg/ml.

FIG. 7 shows the monoclonal rat anti-mouse IL-10 IgM antibody (A), but not monoclonal rat anti-mouse B220 IgM antibody (B), neutralizes the inhibition of IFN-γ secretion by non-cytotoxic, CD8, anti-OFA T cell clone culture supernatants. The data are presented as mean IFN-γ concentrations±SEM for 3 repeats of the experiment. The supernatants from RFM non-cytotoxic CD8 T cell clones 9, 10, and 11 and T_{C} clone 4 were collected 1 week after restimulation of those clones with irradiated 5T lymphoma cells+irradiated, T cell-depleted, RFM spleen cells+IL-2 and added at 10% (v/v) final concentration to cultures of RFM OFA-specific CD4 T cell clone 4 during its restimulation with irradiated 5T lymphoma cells+irradiated, T cell-depleted RFM spleen cells+IL-2.

FIG. 8 shows the monoclonal rat anti-mouse IL-10 IgM antibody (FIG. 8A), but not monoclonal rat anti-mouse B220 IgM antibody (FIG. 8B), neutralizes the inhibition of anti-5T cytotoxicity of T_{C} clone 1 by non-cytotoxic, CD8, anti-OFA T cell clone culture supernatants. The data are presented as mean % specific cytotoxicity±SEM for 3 repeats of the experiment. The supernatants from non-cytotoxic T cell clones 9, 10, and 11 and T_{C} clone 4 were added at 10% (v/v) final concentration. The effector cell:target cell ration was 50:1 and the culture supernatants were added to 5% (v/v) final concentration.

FIG. 9 shows the non-cytotoxic, CD8⁺, anti-OFA T cell clones become able to lyse RFM 5T lymphoma cells if monoclonal anti-IL-10, but not anti-B220, IgM is added for 24 hours before and during the cytotoxicity assay. The data are presented as mean % specific cytotoxicity±SEM for 3 repeat experiments. The effector cell:target cell ratio was 50:1 and the anti-IL-10 or anti-B220 IgM was added to a final concentration of 10 μg/ml.

FIG. 10 shows the macrophages in the cytotoxic clone 1 culture are not the targets for the non-cytotoxic supernatant inhibition of T_{C} clone activity. 24 hours before the regular 2 week restimulation of cytotoxic clone 1 by irradiated 5T lymphoma cells, the cells are harvested and treated with rat anti-mouse CD11b antibody+anti-rat IgG+complement to deplete any macrophages still present or with normal rat IgG+anti-rat IgG+complement as an isotype control antibody. The remaining cells are then cultured for 24 hours in IMDM containing 25% (v/v) final concentration of cytotoxic clone 4 supernatant or noncytotoxic clones 9, 10, or 11 supernatant. After the 24 hour incubation, the cells are washed in IMDM and assayed for anti-5T cytotoxic activity. This was repeated 3 times and the data represent the mean±SEM of % specific cytotoxicity.

FIG. 11. Expression of OFA by breast carcinoma patients' autologous tumor cells. The patients' breast carcinoma cells were tested for their ability to absorb monoclonal anti-OFA IgM 115 before addition of the antibody to an indirect ELISA assay using recombinant OFA/iLRP-conjugated plates. The data shown represent the mean μ the S.E.M. inhibition of maximal reaction by absorption with the tumor cells in 3 repeat assays.

FIG. 12. The surface antigen phenotype of the tumor-reactive T cells cloned from the 4 breast carcinoma patients. Monoclonal anti-CD4, anti-CD8, anti-CD3, anti-TCR Ab, and anti-TCR+facilitating Ab+low toxicity rabbit complement (for use with human cells)-mediated killing of breast carcinoma patient T cell clones was analyzed.

FIG. 13. Proliferative response of the patients' CD4+ T cell clones to 75 ng/well of purified oncofetal antigen bound to nitrocellulose particles. 10,000 viable cloned T cells taken 2 weeks after their latest restimulation with irradiated autologous breast carcinoma cells were incubated with 5×10⁵ irradiated autologous peripheral blood mononuclear cells+75 ng/well of purified RFM mouse 5T lymphoma 44 kD OFA conjugated to nitrocellulose particles (solid bars), 75 ng/well of purified normal thymus 44 kD protein (p44) conjugated to nitrocellulose particles (hatched bars), or an equivalent amount of bare nitrocellulose particles (open bars) for 24 hours, pulsed for an additional 24 hours with 5-bromodeoxyuridine and then assayed for BUDR incorporation using monoclonal anti-BUDR antibody on the cells after fixation in an ELISA assay.

FIG. 14. Proliferative response of the patients' CD8+ T cell clones to 75 ng/well of purified oncofetal antigen bound to nitrocellulose particles. 10,000 viable cloned T cells taken 2 weeks after their latest restimulation with irradiated autologous breast carcinoma cells were incubated with 5×10⁵ irradiated autologous peripheral blood mononuclear cells+75 ng/well of purified RFM mouse 5T lymphoma 44 kD OFA conjugated to nitrocellulose particles (solid bars), 75 ng/well of purified normal thymus 44 kD protein (p44) conjugated to nitrocellulose particles (hatched bars), or an equivalent amount of bare nitrocellulose particles (open bars) for 24 hours, pulsed for an additional 24 hours with 5-bromodeoxyuridine and then assayed for BUDR incorporation using monoclonal anti-BUDR antibody on the cells after fixation in an ELISA assay.

FIG. 15. Secretion of IFN-γ, IL-4, and IL-10 by breast carcinoma patients' OFA-reactive, CD4+ T cell clones upon antigen stimulation. CD4+ clones taken 2 weeks after their most recent restimulation with irradiated autologous tumor cells were cultured for 48 hours with irradiated, T cell-depleted autologous peripheral blood mononuclear cells μ irradiated autologous tumor cells in complete RPMI-1640 medium containing 100 U/ml of recombinant human IL-2. Culture supernatants from those cultures were then harvested and assayed for human IFN-γ, human IL-4, and human IL-10 by quantitative ELISA assays. Data are shown as pg/ml of cytokine secreted by each clone. A) Cytokine secretion subsequent to culture with irradiated autologous T-cell depleted PBML+irradiated autologous breast carcinoma cells; B) Cytokine secretion subsequent to culture with irradiated autologous T cell-depleted PBML in the absence of autologous breast carcinoma cells.

FIG. 16. Secretion of IFN-γ, IL-4, and IL-10 by breast carcinoma patients' OFA-reactive, CD8+ T cell clones upon antigen stimulation. CD8+ clones taken 2 weeks after their most recent restimulation with irradiated autologous tumor cells were cultured for 48 hours with irradiated, T cell-depleted autologous peripheral blood mononuclear cells μ irradiated autologous tumor cells in complete RPMI-1640 medium containing 100 U/ml of recombinant human IL-2. Culture supernatants from those cultures were then harvested and assayed for human IFN-γ, human IL-4, and human IL-10 by quantitative ELISA assays. Data are shown as pg/ml of cytokine secreted by each clone. A) Cytokine secretion subsequent to culture with irradiated autologous T-cell depleted PBML+irradiated autologous breast carcinoma cells; B) Cytokine secretion subsequent to culture with irradiated autologous T cell-depleted PBML in the absence of autologous breast carcinoma cells.

FIG. 17. All CD8+, OFA-reactive clones from patients MP and EP are cytotoxic to their autologous tumor cells, but the IL-10-secreting clones become cytotoxic only in the presence of neutralizing anti-IL-10 antibody. Cytotoxic activity against autologous and allogeneic breast carcinoma cells at a 50:1 effector:target ratio using OFA-reactive CD8 T cell clones from breast carcinoma patients MP and EP in the presence of 10 μg/ml of normal mouse IgG (A) or monoclonal mouse anti-human IL-10 IgG1 (B).

FIG. 18. Proliferation dose response of Patient JR's OFA-reactive CD4+ or CD8+ T cell clones to purified 5T thymic lymphoma 44 kD OFA, purified normal thymus 44 kD protein, or immature laminin receptor protein conjugated to nitrocellulose particles as measured by ELISA determination of BUdR incorporation. Response to OFA (closed circle and closed square), control normal thymus 44 kD protein (open square and open diamond), or iLRP (closed and open triangles) of OFA-reactive CD4 and CD8 clones taken 2 weeks after their latest restimulation with irradiated autologous tumor cells. They were cultured for 48 hours with irradiated autologous PBML and various doses of purified 44 kD OFA, recombinant iLRP, or purified normal thymus p44 in the presence of 100 U/ml of recombinant human IL-2. A, Response of CD4 clones. B, Response of CD8 clones.

FIG. 19. Proliferation dose response of an RFM mouse OFA-specific TC , TH1, and IL-10-secreting TS clone to purified 5T thymic lymphoma 44 kD OFA, purified normal RFM thymus 44 kD protein, various purified recombinant immature laminin receptor protein preparations and a number of control proteins conjugated to nitrocellulose particles as measured by ELISA determination of BUdR incorporation. Response of the 3 clones taken 2 weeks after their latest restimulation with irradiated RFM 5T thymic lymphoma cells and irradiated syngeneic spleen cells in the presence of 100 U/ml of recombinant murine IL-2. A) Response of TC Clone 1, B) Response of TH1 Clone 7, C) Response of TS Clone 9.

FIG. 20. Titration of serum from BALB/c mice immunized with 1 or 10 μg of Immature Laminin Receptor Protein conjugated to nitrocellulose or bare nitrocellulose against iLRP as measured by ELISA A405.

FIG. 21. Western immunoblot of purified OFA, iLRP, and NP40 soluble fraction of mouse thymocytes. Proteins were first electrophoresed on SDS-PAGE, transferred to nitrocellulose membrane and then probed with biotinylated murine anti-iLRP polyclonal antibody as described in Materials and Methods. Lane 1, OFA (5 μg); lane 2, riLRP (5μg) and lane 3, NP40 soluble fraction of thymocytes (50 μg). Arrow shows the position of the reactive band.

FIG. 22. The frequency of CD4+, CD8+, TCR+ and TCR+ T cell clones that are reactive to MCA1315 tumor cells from spleens of mice immunized with bare nitrocellulose, 1 μg iLRP:NC, or 10μg iLRP:NC particles. Monoclonal anti-CD4, anti-CD8, monoclonal anti-TCR, and monoclonal anti-TCR AB+facilitating AB+low toxicity rabbit complement (for use with mouse cells)-mediated killing of immunized mouse spleen T cell clones was analyzed.

FIG. 23. The proliferative response to 75 ng/well of purified recombinant immature laminin receptor protein, purified RFM 5T thymic lymphoma 44 kD OFA, purified normal RFM thymus 44 kD protein conjugated to nitrocellulose particles or bare nitrocellulose particles by CD4 clones established from BALB/c mice immunized with 1 μg, 10 μg or no iLRP:NC particles or bare nitrocellulose. The clones were cultured in the presence of irradiated syngeneic spleen cells in complete RPMI-1640 medium+100 U/ml of recombinant murine IL-2. For the last 24 hours, BUdR was added and BUdR incorporation was measured by ELISA using a monoclonal anti-BUdR antibody+a horseradish peroxidase-conjugated facilitating antibody and substrate. A450 was measured.

FIG. 24. The proliferative response to 75 ng/well of purified recombinant immature laminin receptor protein, purified RFM 5T thymic lymphoma 44 kD OFA, purified normal RFM thymus 44 kD protein conjugated to nitrocellulose particles or bare nitrocellulose particles by CD8 clones established from BALB/c mice immunized with 1 μg, 10 μg or no iLRP:NC particles or bare nitrocellulose. The clones were cultured in the presence of irradiated syngeneic spleen cells in complete RPMI-1640 medium+100 U/ml of recombinant murine IL-2. For the last 24 hours, BUdR was added and BUdR incorporation was measured by ELISA using a monoclonal anti-BUdR antibody+a horseradish peroxidase-conjugated facilitating antibody and substrate. A450 was measured.

FIG. 25. Secretion of IFN-γ, IL-4, and IL-10 by immature laminin receptor protein-immune or nitrocellulose-injected control BALB/c mouse CD4 T cell clones that are reactive to OFA/iLRP upon antigen stimulation. CD4+ clones taken 2 weeks after their most recent restimulation with irradiated syngeneic MCA1315 fibrosarcoma tumor cells were cultured for 48 hours wit irradiated, T cell-depleted syngeneic spleen cells+irradiated MCA1315 cells in complete RPMI-1640 medium containing 100 U/ml of recombinant murine IL-2. Culture supernatants from those cultures were then harvested and assayed for murine IFN-γ, murine IL-4, and murine IL-10 by quantitative ELISA assays. Data are shown as pg/ml of cytokine secreted by each clone μ S.E.M.

FIG. 26. Secretion of IFN-γ, IL-4, and IL-I 0. by immature laminin receptor protein-immune or nitrocellulose-injected control BALB/c mouse CD8 T cell clones that are reactive to OFA/iLRP upon antigen stimulation. CD8+ clones taken 2 weeks after their most recent restimulation with irradiated syngeneic MCA1315 fibrosarcoma tumor cells were cultured for 48 hours wit irradiated, T cell-depleted syngeneic spleen cells+irradiated MCA1315 cells in complete RPMI-1640 medium containing 100 U/ml of recombinant murine IL-2. Culture supernatants from those cultures were then harvested and assayed for murine IFN-γ, murine IL-4, and murine IL-10 by quantitative ELISA assays. Data are shown as pg/ml of cytokine secreted by each clone μ S.E.M.

FIG. 27. Determination that the CD8 T cell clones from the immature laminin receptor protein-immune BALB/c mice were the cells responsible for the IFN-γ and IL-10 secretion. CD8 clones taken 2 weeks after their most recent restimulation with irradiated syngeneic MCA1315 fibrosarcoma cells were cultured for 5 days with irradiated, T cell-depleted syngeneic spleen cells+irradiated MCA1315 cells in complete RPMI-1640 medium containing 100 U/ml of recombinant murine IL-2. At the end of that culture, the cells were split and T cells were positively selected by panning on anti-CD3-coated Petri plates. The CD3 (non-T cell) population and the CD3+ (T cell) population was collected, washed and they and unselected cells continued culture for 48 hours in complete RPMI-1640 medium containing 100 U/ml of recombinant murine IL-2. The supernatants were collected and assayed by quantitative ELISA for IFN-γ (A) and IL-10 (B). The results are presented as pg/ml of cytokine μ S.E.M.

FIG. 28. All CD8 clones from immature laminin-receptor protein-immune BALB/c mice are cytotoxic against OFA+BALB/c MCA1315 fibrosarcoma cells, but the IL-10-secreting CD8 clones can kill only after being incubated in the presence of monoclonal anti-IL-10 for 24 hours before and during the cytotoxicity assay. Cytotoxic activity against MCA1315 fibrosarcoma cells at a 50:1 effector:target ratio in the presence of 10 μg/ml of monoclonal anti-murine IL-10 or rat IgM as an isotype control.

FIG. 29. The cytotoxic activity of IFN-γ- or IL-10-secreting CD8+ T cell clones from immature laminin receptor protein-immune BALB/c mice is specific for OFA and is MHC-restricted. Cytotoxic activity against OFA+, syngeneic MCA1315 fibrosarcoma cells, OFA normal syngeneic spleen cells, and OFA+, allogeneic RFM 5T lymphoma cells was measured at a 50:1 effector:target ratio in the presence of 10 μg/ml of monoclonal anti-IL-10 antibody.

FIG. 30 shows the deduced a.a. sequence (SEQ ID NO:1) of 67LR cDNA showing the sequence of the two peptides (residues 18-40 and residues 43-52) isolated from mAb115-affinity purified OFA (underlined). The sequence of the peptide (residues 64-80) isolated from mAb-affinity purified P44 is shown in bold letters. MALDI-TOF mass spectrometry of trypsin digested P44 revealed proteolytic fragments entirely consistent with the predicted LRP a.a. sequence which covered 67% of the sequence length. Portions of the protein for which corresponding peptides were identified are shaded.

FIG. 31 shows a ELISA binding assay for the anti-OFA monoclonal antibodies (38.46, 38.7, 69.1, 115) to iLRP. MOPC-104E is the IgM-isotype control.

FIG. 32. Western blot showing the binding of the anti-OFA monoclonal antibodies [mAb 38.46 (lane 2), 38.7 (lane 3), 69.1 (lane 4) and 115 (lane 5)] to iLRP (position indicated by arrow). MOPC-104E (lane 1) is IgM-isotype control. Molecular weight markers are indicated.

FIG. 33. Inhibition ELISA. iLRP specifically inhibits the binding of four OFA-specific monoclonal antibodies to biotinylated OFA. 96-well plates were coated with goat anti-mouse IgM antibodies (300 ng/100 μl/well) and incubated with 100 μl of a predetermined amount of the anti-OFA mAbs: 38.46 (hatched columns), 38.7 (wide cross-hatched columns), 69.1 (narrow cross-hatched columns) and 115 (black columns). Several different concentrations of iLRP (31 ng-2 μg) and a fixed amount of biotinylated OFA were then incubated with each antibody as described in “Materials and Methods”. Each bar shows percentage inhibition of control,(mean±SEM, n=3).

FIG. 34. Expression of OFA by MCA1315 murine fibrosarcoma cells and its inhibition by iLRP. Surface expression of OFA on MCA1315 cells was determined by immunostaining with the OFA-specific mAb 69.1 and flow cytometry (left panel). Incubation of the mAb 69.1 with iLRP before immunostaining decreases the intensity of the fluorescence drastically (right panel). White: Cells stained with mAb 69.1. Black: Cells stained with an isotype control mAb (MOPC-104E). Results are expressed as log fluorescence intensity (at 488 nm) in arbitrary units versus relative cell numbers.

### DETAILED DESCRIPTION

It will be apparent to one skilled in the art that various substitutions and modifications may be made to the invention disclosed herein without departing from the scope and spirit of the invention.

As used herein, the term “oncofetal antigen” or “OFA” refers to an antigen which is normally expressed in embryonic and fetal tissue as phase-specific, developmentally regulated, embryonic antigen. The term embraces the 44-kD OFA-associated glycoprotein obtained from membrane extracts of fetal cells and tumor tissues of humans and rodents (species conserved) by monoclonal antibody capture, and the 37 kDa proteinaceous component thereof, and antigenically active fragments thereof. These OFAs are also capable of eliciting a T cell immune response. By “iLRP” it is meant 32-37 kDa immature laminin receptor protein as described in the working examples, below. OFA and iLRP are equivalent for purposes of the present invention.

As used herein, the term “tumor-specific transplantation antigen” or “TSTA” refers to individually specific noncross-protective tumor specific transplantation antigens.

As used herein, the term “tumor-associated transplantation antigen” or “TATA” refers to cross-protective tumor associated transplantation antigens. For example, oncofetal antigen TATA is found in tumors of chemically-, virally- or radiation-induced tumors of rodents and man.

As used herein the term “CD4 effector cells” refers to a subset of T cells which are associated with cell-mediated immune response. They are characterized by the secretion profiles and IFN-γ.

As used herein the term “CD8 effector cells” refers to a subset of T cells which express CD8 on their surface, are MHC class I-restricted, and function as cytotoxic T cells.

As used herein the term “OFA-specific T cell clones” refers to clones which are stimulated to proliferate by recognition of OFA peptide(s) bound to syngeneic MHC class I or class II proteins on the surface of antigen-presenting cells. These clones also are induced to secrete gamma-interferon, IL-2, and in some cases IL-10 upon recognition of OFA peptide(s) presented to them on MHC class I or class II proteins on syngeneic antigen presenting cells.

As used herein the term “anti-OFA T cell receptors” refers to the αβ or γδ T cell receptors which specifically recognize OFA peptide(s) associated with syngeneic class I or class II MHC proteins.

As used herein the term “non-cytotoxic CD8⁺ T cell” refers to CD8-expressing T lymphocytes which recognize and are stimulated to proliferate by some tumor antigen (e.g., OFA) peptide(s) presented by class I MCH proteins on the tumor cell, but cannot kill the tumor cells with which they interact. In some cases, this is because they secrete Interleukin-10 which inhibits their cytotoxic activity.

As used herein the term “cytotoxic CD8 T cell” refers to CD8-expressing T lymphocytes which recognize and are stimulated to proliferate by some tumor antigen (e.g., OFA) peptide(s) presented by class I MHC proteins on the tumor cell. These CD8 T cells kill the tumor cells with which they interact, but can be inhibited from doing so by exogenous IL-10.

As used herein the term “peripheral blood lymphocytes” or “PBLs” refers to lymphocytes in an animal's circulating blood.

As used herein the term “tumor infiltrating lymphocytes” or “TILs” refers to lymphocytes found within and around a tumor which presumably recognize some tumor antigen or peptides of it combined with class I or class II MHC proteins on the tumor cell. They are part of an immune response against the tumor, but some those TILs may be inhibitory to potentially protective immune responses. Some are CD4 and CD8 effector cells.

As used herein the term “antigen processing cells” refers to cells which take up proteins and process them into small peptides (8-9 amino acids) to be presented to T cells via the major histocompatibility molecules.

As used herein the term “intrathymic challenge assay” refers to an assay for thymoma pretumor cells in which subsequent to fractionated, sublethal, whole-body x-irradiation, graded doses of thymus cells from one strain of mouse are injected into the thymus of nonirradiated congenic mice which differ only in a T lymphocyte marker allele. Thus, thymic tumors that develop can be tested for that T cell marker allele to determine if the tumor arose from the donor thymocytes or from the recipient mouse thymocytes. By giving graded doses, one can determine the number of pre-malignant thymocytes in the donor thymus.

As used herein the term “RFM[UnCr mice” refers to a strain of mice bred at Charles Rivers Breeding Laboratories that have the H-2f MHC genotype, are albino, and which develop thymic lymphoma/leukemia subsequent to fractionated, sublethal whole-body X-irradiation. RFM is the strain name.

As used herein the term “5T” refers to the radiation-induced Lymphoblastic Lymphoma cell line XR11-5T isolated from the thymus of RF/M mouse. This cell line is of thymic origin and L3T4⁺Lyt-2⁺, and Thy-1⁺.

As used herein the term “IFN-γ” refers to an abbreviation for gamma interferon (or interferon-γ). Gamma interferon is a cytokine produced and secreted by activated T lymphocytes. It can protect cells from becoming infected with virus. It also can enhance MHC class I and II expression on B lymphocytes and macrophages, and at higher levels induces class II on many tissue cells to enhance antigen presentation. It increases IL-2 receptors on cytotoxic T lymphocytes, enhances cytotoxic activity of large granular lymphocytes and promotes B cell differentiation to IgG-producing cells. Gamma interferon is the principle cytokine responsible for macrophage arming factor activity which increases macrophage Fc receptor expression on macrophages as well as inducing the macrophages' respiratory burst, thereby enhancing their ability to kill infecting microbes as well as tumor cells. It can inhibit proliferation of Th2 CD4 T cells (T helper cells for antibody production). It is a marker cytokine for the CD4 effector T cells.

As used herein the term “IL-10” refers to a cytokine produced by a number of cell types including T lymphocytes and macrophages. Interleukin-10 can promote the growth and activation of some immune cells, but it is secreted by CD4 Th2 cells and inhibits activation of Th1 cells and especially inhibits their secretion of gamma interferon. It acts mostly through antigen-presenting cell inhibition, but the inventors of the present invention have shown that it inhibits antitumor cytotoxic T cell activity directly.

As used herein the term “Tc clone cell” refers to T lymphocytes which have been cloned from peripheral blood, spleen, lymph node, or from tumor-infiltrating lymphocytes. A clone of this type is cytotoxic for tumor cells and usually expresses CD8 and recognizes some tumor antigen peptide bound to autologous (or syngeneic) class I MHC proteins. It is specific in its killing in that it only kills those cells which express the tumor antigen peptide(s) on the tumor cells' class I MHC molecules. In the studies developing the present invention, they also secrete gamma interferon upon stimulation by the tumor cells or the tumor cell antigen peptide(s).

As used herein the term “MCA1315” refers to fibrosarcoma cells induced into the tumorigenic state by subcutaneous injection of BALB/c mice with methylcholanthrene (MCA). Tumor cell lines are then isolated and the different isolates are given Ser. Numbers.

As used herein the term “IMDM” refers to Iscove's Modified Dulbecco's Medium.

As used herein the term “ELISA” refers to the Enzyme Linked Immunosorption Assay.

The use and methods of preparation of oncofetal antigen or oncofetal antigen specific monoclonal antibodies for human and animal cancer detection, therapy, and therapy monitoring is disclosed in U.S. Pat. No. 4,686,180. A 44 kDa oncofetal antigen glycoprotein (gp) and a 200 kDa glycoprotein, possibly containing the 44 kDa component, have been shown to be a species-conserved, cell surface associated glycoprotein which serve as embryo-fetal and cancer specific antigens and immunogens in inbred pregnancy and in primary rodent cancer models. Oncofetal antigens are present in early and mid-gestation rodent and human fetus and are consistently re-expressed in tumor tissue, but are not present in normal term, neonate, or adult tissues.

A first embodiment of Applicants' invention is directed to a method of screening an individual for cancer or monitoring cancer therapy in a cancer patient. Such cancers include carcinomas, hematologic cancers and sarcomas arising from the 3 germ layers. The method is equally well suited for individuals suspected of having or presenting with cancer, post-surgical patients and patients undergoing cancer therapy such as chemotherapy, immunotherapy and/or radiation.

The test can be qualitative or semi-quantitative in nature, and entails obtaining a sample of T-lymphocytes from an individual and analyzing the sample for the presence or relative frequency of various OFA- or iLRP-specific T cell subclasses, e.g., by limiting dilution cloning analysis. In preferred embodiments, the sample is obtained from peripheral blood lymphocytes or tumor infiltrating lymphocytes. Separating the T-cells from other components of blood and other tissue such as non-lymphocytic cells, macrophages and non-cellular components, is conducted by standard techniques, such as the dilution of heparinized blood in growth medium and separation over Ficoll-Paque Plus by centrifugation and collecting the lymphocytes at the interface. It is preferred that the separated T cell sample is substantially free of the aforementioned substances, but it is not necessary. To conduct the semi-quantitative test, the T lymphocytes are cloned said lymphocytes, typically by diluting the cells and plating single cells onto different microtiter plate wells. The cloned lymphocytes comprise memory CD4 helper cell subclasses, CD8 cytotoxic T-lymphocyte subclasses and CD8 non-cytotoxic T-suppressor lymphocyte subclasses. The cloned lymphocytes are then contacted with (or exposed to) a composition containing oncofetal antigen (OFA) or immature laminin receptor protein (iLRP), preferably in purified form, which stimulates OFA- or iLRP-specific T-cells to proliferate. This procedure will generate relative frequencies of memory CD4 helper cells, CD8 cytotoxic T lymphocytes and CD8 non-cytotoxic T-suppressor lymphocytes. The amount of OFA or iLRP added to the T lymphocyte clones generally ranges from about 15-75 ng, up to about 10 ug per well in microtiter plates. Complexing the OFA or iLRP with a carrier (e.g., nitrocellulose or biologically inert particles such as latex), or adding an adjuvant enhances the re-stimulation effect and/or the processing of the “antigen” by antigen processing cells.

Following re-stimulation, the frequency of each of the OFA- or iLRP-specific T lymphocyte subclasses relative to each other is determined. A higher frequency of OFA- or iLRP-specific CD8 cytotoxic T lymphocytes compared to non-cytotoxic T suppressor lymphocytes is indicative of cancer cell destruction and tumor regression in the host, whereas a higher frequency of CD8 T suppressor cells indicates the inhibition of T cytotoxic lymphocytes destruction of cancer cells by the IL-10 produced by the T suppressor cells. In addition, the presence or relative frequency of T lymphocyte subclass known as CD4 Th1 cells may also be determined as they contribute to either direct or recruited tumor resistance, once they have been stimulated by 44 kD OFA. These cells arouse circulating macrophages which will kill tumor cells non-specifically. In regard to therapy monitoring, a relatively high frequency of the CD8 cytotoxic T cells indicates that therapy is effective and the possibility of remission is high, whereas a relatively low frequency of these cells compared to non-cytotoxic T suppressor cells indicates that therapy is ineffective and prognosis is poor. A variety of techniques may be employed. One technique involves flow cytometry, in which case the tumor-expressed OFA or iLRP is established and the subclasses of cloned T-cells are phenotyped. Another technique involves placing autologous living tumor cells of the patient in growth medium and adding CD4 or CD8 OFA- or iLRP-specific T-cell subclasses to the wells and detecting target cell killing in vitro. Yet another technique utilizes ELISA, in which case detectably labeled anti-OFA/iLRP antibodies bind OFA/iLRP on tumor target cells from the patient's biopsy to identify OFA/iLRP present. The more OFA/iLRP detected, the greater the frequency of T suppressor cells, which in the case of a cancer patient undergoing therapy is indicative of a large, fast growing tumor. ELISA is also used to quantitate the OFA/iLRP content in purified samples used to re-stimulate the T-lymphocytes. Yet another technique entails Western blot analysis which detects the molecular weight of OFA/iLRP present. A further technique involves re-stimulation and limited dilution cloning in vitro to generate specific OFA-activated T-cell subclones.

The qualitative test can be done faster, but it does not provide quantitative frequency data. To practice this method, the T lymphocytes are stimulated by the addition of the OFA/iLRP composition first, and then cloned. This sequence of steps for the determination of the presence of CD8 cytotoxic T lymphocytes relative to non-cytotoxic T-suppressor lymphocytes. A particularly preferred technique of distinguishing CD8 cytotoxic T-lymphocytes from CD8 T suppressor cells in a sample containing T cells in this method involves contacting the sample (contained in a suitable growth medium) with a detectably labeled anti-gamma interferon antibody. CD8 cytotoxic T lymphocytes secrete gamma interferon so binding of the antibody indicates presence of CD8 cytotoxic T lymphocytes in the sample.

Following separation from the blood or tissue sample, the T lymphocytes are cultured in a T cell growth medium comprising at least one cytokine, e.g., interleukin-2 and/or interleukin-6, and autologous antigen processing cells. These cells may be obtained from mononuclear T-lymphocytes left over in the unseparated T-cell fraction. They are lethally irradiated prior to addition to the medium. They bind anti-CD3 monoclonal antibodies. Interleukin-2 generates stable CD4 T-cell clones; interleukin-6 generates stable CD8 T cell clones; and autologous antigen processing cells, which have a CD3 marker and can be obtained from the same sample, process memory T cell precursors. Gamma-interferon is also added, as it inhibits the cloning of CD4 Th2 cells which should be avoided. A preferred T cell growth medium is RPM-1640. It is also preferred that the T cells are stimulated and cloned in this medium.

Another and related aspect of Applicants' invention is directed to a kit for conducting the method for detecting cancer or monitoring cancer therapy. The elements of the kit typically contain a cytokine which is interleukin-2 or interleukin-6, gamma-interferon, a T-lymphocyte growth medium, autologous antigen processing cells, preferably irradiated, 44 kD OFA or immature laminin receptor protein, at least one reagent for measuring DNA stimulation; and CD4, CD8 and interleukin-10 phenotyping reagents. As disclosed above, in preferred embodiments, the OFA/iLRP is preferably in purified form and is in admixture with an adjuvant or complexed with a carrier or support.

In addition, the presence of IL-10 or IL-10 mRNA in CD8+ T cells, which in preferred embodiments, is detected with two and three color fluorescence in fixed and permeabilized T-cell flow cytometry using either in peripheral blood lymphocytes or in tumor infiltrating lymphocytes in the tissues of the residual tumor bed, indicates a strong potential for tumor promotion and cancer regrowth. Thus, detecting IL-10 levels in -culture supernatants of clonally expanded T-cells or in tumor infiltrating lymphocyte-containing biopsy tissues taken from the tumor bed provides yet another technique for distinguishing the various clones of T cell subclasses and monitoring the progression of disease or the effectiveness of therapy. A kit for this method of measuring IL-10 levels would also include an anti-IL-10 monoclonal antibody or probes specific for detecting IL-10 mRNA.

The present invention discloses that the inhibitory substance secreted by the non-cytotoxic CD8 T cell clones can inhibit T cell secretion of interferon-γ, is not antigen-specific, and is not MHC-restricted. The inhibitory substance, however, is neutralized by anti-IL-10 monoclonal antibody but not by an isotype control antibody. Also, the supernatants of these antigen-restimulated, non-cytotoxic CD8 T cells contain IL-10, while the supernatants of antigen-restimulated, cytotoxic CD8 T cell clones do not. The present invention thus also discloses that inclusion of anti-IL-10 antibody in the cultures of the non-cytotoxic CD8 T cell clones, rescues their anti-tumor cytotoxic ability. Further, it is shown that the IL-10 does not come from macrophages or tumor cells, but from the clones. Macrophages are not the targets of the inhibitory activity, but appear to act on the T_{C} clone cells. Thus, the present invention demonstrates that CD8 T cells take on the functional activity of “suppressor” T cells for cell-mediated immunity by having the gene for IL-10 activated and the secretion of that cytokine can mask the functional potential of the secreting T cell itself.

It has been reported that in irradiated, long-term surviving RFM mice there is enhanced kinetics of tumor development upon challenge with RFM lymphoma cells. Splenic OFA-specific, non-cytotoxic, CD8⁺ T cells from such mice were cloned. Upon antigen stimulation, these non-cytotoxic CD8⁺ T cell clones secrete a factor that inhibits the ability of OFA-specific RFM T_{C} cell clones from killing 5T RFM lymphoma cells in vitro. The supernatants from non-cytotoxic, CD8⁺ T cells do not inhibit the tumor cell-induced proliferation of the T_{C} cell clones, however. The present invention demonstrates that OFA-stimulated, non-cytotoxic, CD8 T cell clone culture supernatants also inhibit interferon-γ secretion by stimulated CD4 and CD8 anti-OFA effector T cell clones in a dose-dependent manner. The inhibitor in those culture supernatants acts neither in an antigen-specific nor MHC-restricted manner. Culture supernatants of OFA-stimulated non-cytotoxic CD8 T cell clones' contain IL-10, while those from OFA-stimulated, RFM OFA-specific T_{C} clones do not. Moreover, the monoclonal anti-IL-10 antibody specifically blocks the inhibition of cytotoxic activity and interferon-γ secretion by OFA-specific CD8 and CD4 effector T cell clones in a dose-dependent manner in vitro. Incorporation of anti-IL-10 antibody into the cytotoxicity assays of the OFA-specific, non-cytotoxic CD8⁺ T cell clones against 5T tumor cells restores their cytotoxic activity.

Accordingly, another embodiment of Applicants' invention is directed to a method for rendering T-suppressor cells cytotoxic, and involves the administration to an individual an agent that inhibits or neutralizes IL-10 production by the T suppressor cells. This limits their ability to secrete IL-10. IL-10 is primarily responsible for the suppression of CD8 and CD4 cytotoxicity at the tumor site or in the peripheral blood of a person stricken with cancer. By decreasing or neutralizing IL-10 produced by T suppressor cells at the tumor site in vivo, the potency of the immune system is greatly enhanced because not only do the CD8 and CD4 T cells retain their cytotoxicity, but the T suppressor cells become cytotoxic as well. Agents that selectively kill CD8 T suppressor cells making IL-10, drugs that inhibit IL-10 synthesis, and substances that neutralize IL-10 activity such as anti-IL-10 antibodies, are useful in this embodiment of the invention.

Oncofetal antigen serves as a Tumor Associated Transplantation Antigen (TATA) in rodent cancer systems representative of all three germ lines giving rise to adult tissues and tumors. Oncofetal antigen or iLRP, in crude or purified form, as identified with oncofetal antigen-specific monoclonal antibodies and OFA-specific T-cells, can promote both B-cell mediated anti-oncofetal antigen antibody production as well as protective, T-cell mediated immunity in syngeneic rodents.

Human lung cancer patients appear to make IgG to oncofetal antigen that is present in the tumors. The antibody was detected by an ELISA absorption procedure with fresh autologuous biopsy material or purified mouse or human oncofetal antigen. Oncofetal antigen or iLRP, delivered in an appropriate dosage and frequency for vaccination, can promote tumor immunity to challenge, as well as prevent the induction of primary tumors in rodents. Oncofetal antigen on fetal cells has been conferred to interrupt chemical carcinogenesis in rats and viral carcinomas in hamsters when used as vaccine. T-cell mediated immune responses are credited with oncofetal antigen associated tumor protection.

The oncofetal antigen or iLRP stimulates and causes the clonal expansion of memory CD4 helper (Th1) and CD8 Tc cytotoxic lymphocytes as well as CD8 non-cytotoxic (Ts) T-suppressor lymphocytes in inbred mice experiencing and subsequently eliminating X-ray-induced lymphomagenesis or 3-MCA sarcoma production. These mice were never presented with oncofetal antigen via direct immunization. The mice immunologically “experienced” oncofetal antigen re-expressed and present on their own primary tumors after or during malignant transformation. The mice were nevertheless sensitized to the oncofetal antigen on their primary tumors and were found to carry oncofetal antigen specific T-cells that could be clonally expanded when stimulated with purified syngeneic or allogeneic mouse oncofetal antigen in culture medium containing specific supplements. 44 kDa oncofetal antigen, in the presence of selected cytokines, stimulates the enrichment of these clones in vitro. Highly stable CD4 and CD8 T-cell clones were thus derived and exhaustively tested for function in vitro. The clones selected as oncofetal antigen specific could functionally “help” as CD4 cells in tumor cell destruction by arousing macrophages or by stimulating expansion of CD8 protective effector cells which could kill autologuous tumor target cells in vitro. Other CD8 clones that arose were not cytotoxic but could ablate CD8 T-cell mediated oncofetal antigen TATA or TSTA specific cytotoxicity in response to the expression of oncofetal antigen on primary X-ray or MCA sarcoma tumor cells.

Accordingly, a further embodiment of the present invention is drawn to a method of stimulating and causing clonal expansion of memory CD4 helper cells, CD8 T_{C} cytotoxic lymphocytes and CD8 non-cytotoxic T-suppressor lymphocytes in vivo comprising administering an effective dose of purified oncofetal antigen or iLRP.

It is specifically contemplated that pharmaceutical compositions may be prepared using the purified oncofetal antigen or iLRP of the present invention. In such a case, the pharmaceutical composition comprises the purified oncofetal antigen or iLRP of the present invention and a pharmaceutically acceptable carrier. A person having ordinary skill in this art would readily be able to determine, without undue experimentation, the appropriate dosages and routes of administration of the OFA or iLRP. When used in vivo for therapy, the purified OFA or iLRP is administered to the patient or an animal in therapeutically effective amounts, i.e., amounts that eliminate or reduce the tumor burden. It will normally be administered parentally, preferably intravenously, but other routes of administration will be used as appropriate. The dose and dosage regimen will depend upon the nature of the cancer (primary or metastatic) and its population, the characteristics of the particular immunotoxin, e.g., its therapeutic index, the patient, the patient's history and other factors. The amount of purified OFA or iLRP administered will typically be in the range of about 0.1 to about 10 mg/kg of patient weight. The schedule will be continued to optimize effectiveness while balanced against negative effects or treatment. See Remington's Pharmaceutical Science, 17th Ed. (1990) Mark Publishing Co., Easton, Penn.; and Goodman and Gilman's: The Pharmacological Basis of Therapeutics 8th Ed (1990) Pergamon Press; which are incorporated herein by reference.

For parenteral administration the protein will most typically be formulated in a unit dosage injectable form (solution, suspension, emulsion) in association with a pharmaceutically acceptable parenteral vehicle. Such vehicles are preferably non-toxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. The purified OFA or iLRP will typically be formulated in such vehicles at concentrations of about 0.1 mg ml to 10 mg ml.

The following examples are given for the purpose of illustrating various embodiments of the invention and are not meant to limit the present invention in any fashion.

### EXAMPLE 1

Mice

RFM/UnCR male and female 6-10 week old mice used in these experiments were obtained through NIH from Charles Rivers Breeding Laboratories (Wilmington, Mass.).

### EXAMPLE 2

Tumor Cells

The RFM thymic lymphoma 5T used for restimulation of clone proliferation (Rohrer, S. D., supra.) was cultured in Iscove's Modified Dulbecco's Medium (IMDM) supplemented with 100 U/ml Penicillin G and 100 μg/ml Streptomycin sulfate, 10% control process serum replacement 3 (CPSR-3) (Sigma Chemical Company, St. Louis, Mo.), 2 mM L-glutamine, and 3.024 g/L sodium bicarbonate. The cells were maintained in a 37° C. humidified 5% CO₂, 95% air atmosphere. The BALB/c fibrosarcoma MCA1315 which was used to restimulate BALB/c anti-oncofetal antigen clones was cultured in the same medium under the same temperature-CO₂ conditions.

### EXAMPLE 3

Cell Lines

The gibbon T cell lymphoma MLA-144 (American Type Culture Collection, Rockville, Md.) constitutively secretes gibbon IL-2 [Rabin, et al., J. Immunol. 127:1952, (1981)], and was cultured in IMDM supplemented with 7.5×10⁻⁵ M α-thioglycerol, 2 mM L-glutamine, sodium bicarbonate (3.024 g/L), 100 U/ml Penicillin G, 100 μg/ml Streptomycin sulfate, and 10% CPSR-3 (Sigma Chemical Company, St. Louis, Mo.) (complete IMDM).

### EXAMPLE 4

Monoclonal Antibodies

Rat monoclonal anti-mouse IL-10 IgM antibody (clone AB-71-005) and rat monoclonal anti-mouse CD11b (Mac-1) (clone M1/70) were purchased from BioSource International (Camarillo, Calif.). Normal rat IgG which was used as a control isotype antibody was purchased from Pharmingen (San Diego, Calif.). Rat monoclonal anti-mouse B220 IgM antibody was purified by ammonium sulfate precipitation and Sephadex G-200 gel filtration from culture supernatants of hybridoma RA3-3A1/6.1. Rat monoclonal anti-mouse CD4 antibody (hybridoma GK1.5) and rat monoclonal anti-mouse CD8 antibody (hybridoma 53-6.72) were purified by ammonium sulfate precipitation and protein G affinity chromatography from culture supernatants. These hybridomas were obtained from the American Type Culture Collection (Rockville, MD) and maintained in the laboratory.

### EXAMPLE 5

T Cell Clone Maintenance

The clones were cultured in sterile IMDM supplemented not only with 100 U/ml of recombinant murine IL-2 and 100 U/ml of recombinant murine. IFN-γ, but also with 10 U/ml of recombinant murine IL-6. Sterile filtered MLA-144 culture supernatant was used as the source of IL-2 (at 25% v/v). The RFM clones were restimulated with irradiated 5T cells and the BALB/c clones were restimulated with irradiated MCA1315 cells every two weeks in the presence of irradiated syngeneic spleen cells and complete IMDM supplemented at 25% v/v final concentration with MLA-144 culture supernatant to maintain the clones' viability and proliferation.

### EXAMPLE 6

Determination of T Cell Clones Cytotoxic T Cell Activity Against 5T Lymphoma Target Cells

Cytotoxicity assays were performed using the CytoTox 96 non-radioactive cytotoxicity assay kit produced by Promega (Fisher Scientific, Atlanta, Ga.). The assay quantitatively measures lactic dehydrogenase (LDH), a stable cytosolic enzyme that is released upon cell lysis. Released LDH in culture supernatants is measured with a 30 minutes coupled enzymatic assay resulting in the conversation of a tetrazolium salt to a red formazan product [Decker, et al., J. Immunol. Methods 15:61, (1988)]. The amount of color formed was proportional to the number of lysed cells. Color was quantitated using a Biotek ELISA reader measuring absorbance at 492 nm. Preliminary experiments determined that use of 10,000 viable 4T or 5T lymphoma cells would allow release of enough LDH upon lysis to give a strong absorbance. Each time a cytotoxicity assay was done, duplicate control wells containing only target cells, only effector cells, or only medium were run to control for spontaneous release of LDH by effector and target cells and for any color provided by the medium itself. Initially, all data had the medium control absorbance value subtracted from them. Duplicate wells containing only medium to which 10 μl of 10× lysis solution was added were used for a volume correction control. The average absorbance of that control was subtracted from the absorbance values obtained from the target cell maximum release wells. The percent specific cytotoxicity was calculated using the formula listed below:

$$\% Cytotoxicity = \frac{( Exp . - Effector Spontaneous ) - Target Spontaneous}{Target Maximum - Target Spontaneous} \times 100$$

This assay has much less spontaneous release of LDH than one gets of ⁵¹Cr in a traditional ⁵Cr release cytotoxicity assay and so higher specific cytotoxicity percents are achieved.

At the time of the two-week restimulation of the clones to maintain their proliferation, the cloned cells were harvested, washed in IMDM, and a viability count was done. A portion of the cells was saved out to be used in the cytotoxicity assay. Into 8 wells of V-bottomed 96 well plates, were placed 200 μl of medium-washed target 5T lymphoma cells such that there were 10,000 cells/well in the target spontaneous release control and the target maximal release control wells. Into 6 wells/clone of V-bottomed 96 well plates were placed 100 μl of medium-washed target 5T lymphoma cells such that there were 10,000 live target cells/well. Into each of two wells/clone was added 100 μl of medium-washed cloned T cells at 12.5 clone cells:l target cell, 25 clone cells:l target cells, or 50 clone cells:1 target cell. These are the experimental wells. Into 6 wells/clone were placed 200 μl of medium-washed cloned T cells at the same concentrations as in the experimental wells except that no target cells are present; These served as the effector spontaneous release wells. The 96 well plates were centrifuged at 250×g for 4 minutes to pellet all cells and then incubated for 4 hours at 37° C. in a humidified, 95% air/5% CO₂ atmosphere. At the end of this incubation, 10 μl of 10× lysis solution/100 μl of medium was added to each of the maximal release wells to lyse the targets. The plates were then continued to be incubated at 37° C. for another 45 minutes. The plates were then centrifuged at 250×g for 4 minutes to pellet remaining cells and 50 μl of culture supernatant from all wells was transferred to a flat-bottomed 96 well ELISA plate. 50 μl of reconstituted substrate mix in assay buffer was then added to each well and the plates were incubated at room temperature for 30 minutes. This substrate solution contained lactate, NAD (nicotinamide-adenine dinucleotide), INT (p-idonitrotetrazolium violet chloride), tetrazolium salt, and the enzyme diaphorase at optimal concentrations for these volumes. 50 μl of stop solution was added to each well, any bubbles were removed and the absorbance at 492 nm wavelength was determined using a Biotek ELISA reader.

### EXAMPLE 7

Determination of Inhibitory Activity of Supernatants from n On-Cytotoxic CD8⁺ T Cell Clones on Interferon-γ Secretion

One day before the required every two week re-exposure of the clones to irradiated 5T tumor cells in the presence of irradiated spleen cells and IL-2, some of the cells from the clones to be tested were harvested, washed three time sin IMDM, and viability counts were performed. The cells were seeded into 24 well plates at 10⁵ viable cells/ml in IMDM containing IL-2±various amounts of culture supernatant taken from non-cytotoxic CD8⁺ clones or from cytotoxic clone 4. The culture supernatants used were obtained one week after re-stimulation with irradiated 5T tumor cells in the presence of irradiated T cell-depleted RFM spleen cells+IL-2. The cytotoxic clones were incubated±the supernatants for 24 hours and then harvested, washed three times in IMDM and counted fro viability. The supernatant-treated clone cells were then restimulated by irradiated 5T lymphoma cells or MCA1315 fibrosarcoma cells in the presence of irradiated T cell-depleted syngeneic spleen cells+IL-2 for 48 hours and the supernatants collected, sterilized by filtration and assayed for interferon-γ by ELISA.

### EXAMPLE 8

ELISA Determination of Interferon-γ Secretion by the T Cell Clones

An interferon-γ assay kit from Genzyme Corp. (Cambridge, Mass. ) was used. Briefly, a 96-well flat-bottomed ELISA plate was coated with monoclonal anti-mouse IFN-γ antibody in coating buffer (0.1 ml/well), the wells sealed with plastic sealant, and incubated overnight in a humidified box at 4° C. The coating solution was aspirated from the wells and each well washed with 200 μl of washing buffer followed by aspiration. This wash was repeated three times. The plate was then blotted dry and 200 μl of blocking/dilution buffer added to each well. The plate was sealed and incubated at 37° C. for 30 minutes. At the end of this incubation, the plate was unsealed and the liquid aspirated from the wells. The 100 μl of medium (negative control) was placed in two wells, 100 μl of recombinant IFN-γ (diluted in medium to 125 to 8200 pg/ml) placed in two wells/concentration (standard curve), and 100 μl of each test sample was placed in two wells/sample. The plate was sealed and incubated at room temperature for 2 hours. After that incubation, the liquid was aspirated from the wells and each well was washed four times with washing buffer at room temperature and the plate blotted dry.

The 100 μl of diluted polyclonal goat anti-mouse IFN-γ antibody was then added to each well and the plate sealed and incubated for 2 hours at room temperature. The liquid was then aspirated from the plate and the plate washed four times with washing buffer and blotted dry. The 100 μl of diluted polyclonal donkey anti-goat Ig antibody that was conjugated with horseradish peroxidase was added to each well, the plate sealed, and incubated at room temperature for 1 hour. The liquid was aspirated from the plate and the plate was washed four times with washing buffer then blotted dry and 100 μl of diluted substrate reagent (OPD chromagen in substrate reagent buffer/peroxide solution) was added to each well. The plate was incubated at room temperature until a faint yellow color was discernible in wells containing 125 pg/ml mouse IFN-γ, which was usually 4 to 6 minutes. At that point, 100 μl of 2 N sulfuric acid was added to each well in the same order as the substrate reagent was added to stop the reaction. The plate was then read in a Biotek ELISA reader measuring absorbance at 492 nm. The average absorbance reading of duplicate wells was determined and the average absorbance of the negative control subtracted from all averages. The average absorbance for each concentration of IFN-γ used in the standards (on the y-axis) was plotted against the concentration of IFN-γ (on the x-axis) on semilog graph paper. The concentration of IFN-γ in the test culture supernatants was determined by using the standard curve that is generated. The standard curve was linear between 250 and 4100 pg/ml.

### EXAMPLE 9

ELISA Determination of IL-10 Secretion by the T Cell Clones

An IL-10 ELISA assay kit from Bio-Source International (Camarillo, Calif.) was used. Briefly, in a 96 well flat-bottomed ELISA plate coated with monoclonal anti-mouse IL-10 antibody was added 100 μl of the standard diluent to the blank and zero wells and 100 μl of standards, experimental supernatants, and controls were added to appropriate wells. The plate was covered and incubated for 1.5 hours in a 37° C. incubator. After that incubation, the liquid was aspirated from the wells and the wells washed 4× with wash buffer. The plate was then inverted and allowed to drain. To all wells except blank wells was then added 100 μl of biotinylated anti-IL-10 antibody. The plate was then covered and incubated at 37° C. for 45 minutes. The liquid was then aspirated and the wells washed 4× with wash buffer and drained. Following that, 100 μl of 1:100 diluted horseradish peroxidase (HRP)-conjugated Streptavidin solution was added to all wells. The plate was then covered and incubated at 37° C. for 45 minutes. The liquid was then aspirated and the wells washed 4× with wash buffer and drained. 100 μl of stabilized TMB chromogen was added to all wells and the plate covered and incubated at room temperature in the dark for 20 minutes. 100 μl of Stop Solution was then added to each well, gently mixed, and the absorbance at 450 nm determined for each well on a BioTek ELISA reader. The blank well contained only the chromogen and stop solution. All wells were done in duplicate. The average absorbance reading of duplicate wells was determined and the average absorbance of the negative control subtracted from all averages. The average absorbance for each concentration of IL-10 used in the standards (on the y-axis) was plotted against the concentration of IL-10 (on the x-axis) on semilog graph paper. The concentration of IL-10 in the test culture supernatants was determined by using the standard curve that was generated. The sensitivity of this ELISA was <13 μg IL-10/ml and the standard curve was linear between 31.2 pg/ml and 2000 pg/ml.

### EXAMPLE 10

Ability of Anti-IL-10 to Block Inhibitory Supernatant Effects on IFN-γ Secretion and Cytotoxicity by T Cell Clones

One day before the two-week restimulation of the RFM T cell clones with 5T lymphoma cells, the cultures were harvested, washed thrice in IMDM and a portion of the cells counted for viability. 2×10⁵ viable cells/ml were seeded into wells in a 24 well plate in IMDM+IL-2. Into most cultures was added culture supernatant from non -cytotoxic, oncofetal antigen-specific CD8 T cell clones 9, 10, or 11 or culture supernatant from cytotoxic CD8 T cell clone 4 to a final concentration of 10% (v/v). To this solution was added various concentrations of monoclonal anti-IL-10 or anti-B220 IgM antibodies and the cultures incubated at 37° C. for 24 hours. At the end of this incubation, the cells were harvested, washed thrice in medium, added to a restimulation culture as described previously (Ragin, et al., supra.) and 48 hours later supernatant was collected and assayed for IFN-γ. Determination of anti-IL-10 blocking of the inhibition of cytotoxicity was done the same way except that after the 24 hour incubation with inhibitory supernatant±anti-IL-10 antibody, the cells were harvested, washed, counted, and put in a cytotoxicity assay as described above.

### EXAMPLE 11

Determination of Anti-IL-10 Conversion of Non-Cytotoxic CD8, Oncofetal Antigen-Specific T Cell Clones to Cytotoxic Clones

In order to determine if the clones that were secreting IL-10 were being inhibited by it, the cells were harvested one day before the two week restimulation culture and set up with 101 μg/ml anti-IL-10 IgM or anti-B220 IgM as described above for 24 hours. The cells were then harvested, washed thrice in IMDM, and viability counts performed. The cells were then added to an anti-5T cytotoxicity assay as described above, except that anti-IL-10 or anti-B220 was added to a final concentration of 10 μg/ml.

### EXAMPLE 12

IL-10 Produced by Non-Cytotoxic T Cell:5T Tumor Cell Cultures during Clone Restimulation was not Due to Tumor Cell IL-10 Production

One week after restimulation of cytotoxic and non-cytotoxic CD8 T cell clones with irradiated 5T cells, the cells were harvested, washed three times in IMDM and the tumor cells separated out by two serial positive selections on anti-mouse CD4-coated sterile bacterial Petri plates and two serial positive selections on anti-mouse CD8-coated plates. A modification of the method of Wysocki and Sato (Decker, et al., supra.) was used in that the antibody was coated on the plates the day of cell separation. After non-adherent cells were gently washed away, sterile PBS was added and the plates agitated, followed by pipetting off the cells attached to the Petri plates. These cells were then washed three times in IMDM, viability counts done, and the tumor cells cultured for 48 hours in IMDM and culture supernatants collected, sterile filtered, and assayed for IL-10 as described above.

The T cell clones were separated from the tumor cells by a combination of negative selection using anti-CD4 antibody+facilitating antibody+low toxicity rabbit complement to remove CD4 T cells (including the 5T cells). The remaining cells were washed three times in IMDM, and positively selected on anti-CD8-coated Petri plates using a modification of the method of Wysocki and Sato [Wysocki, et al., Proc. Natl. Acad. Sci. USA 75:2844, (1978)] as described above. The extent of depletion and enrichment were determined by immunofluorescent microscopy analysis. The resulting CD4⁻, CD8⁺ T cells were cultured in IMDM+recombinant IL-2 for 48 hours and the supernatant was sterile filtered and assayed for IL-10 by ELISA. The 5T lymphoma cells and the T cell clones could be separated because the 5T tumor cells are CD4+, CD8⁺ T cells (Coggin, et al., supra.) while the clones are CD4⁻, CD8⁺ T cells (Payne, et al., supra.).

### EXAMPLE 13

Macrophages were Neither the Source nor the Target of the IL-10

To show that macrophages, which were in the clone restimulation cultures, were not the source of the IL-10 subsequent to 5T restimulation of non-cytotoxic CD8 T cells, the T cell clone cultures were harvested 1 week after restimulation with irradiated 5T lymphoma cells+irradiated T cell-depleted spleen cells. The clone cells were serially negatively and positively selected for CD4 and CD8 as described above or the cells were treated with anti-CD11b+anti-rat IgG antibody+low-toxicity rabbit complement to eliminate macrophages and the selected cell subpopulations separately were cultured for 48 hours in IMDM+recombinant IL-2. The culture supernatants were then harvested and sterile filtered. IL-10 was assayed by ELISA as described above.

Similarly, to determine that macrophages were not the target of the IL-10, anti-oncofetal antigen CD8⁺ cytotoxic T cell clone 1 cultures were harvested one day before the required 5T restimulation of the clones and the cells washed three times in medium and treated with rat anti-mouse monoclonal CD11b antibody+anti-rat IgG+low toxicity rabbit complement (to eliminate macrophages) or with rat IgG isotype control antibody+anti-rat IgG+low toxicity rabbit complement. The remaining cells were washed three times with IMDM and treated for 24 hours with supernatants from non-cytotoxic clones 9, 10, or 11 or from cytotoxic T cell clone 4 as described previously (Rohrer, J. W., et al., supra.). The clone cells were then assayed for anti-5T cytotoxicity as described above.

### EXAMPLE 14

Statistical Analysis of Data

Most data were analyzed for significant differences using Student's t-test. The data from experiments in which dose response curves were generated were analyzed using Analysis of Variance. A p value<0.05 was considered significant.

### EXAMPLE 15

Supernatants from Non-Cytotoxic, Anti-OFA CD8 T Cell Clones Inhibit Interferon-γ Secretion by Anti-OFA CD4 and CD8 T Cell Clones

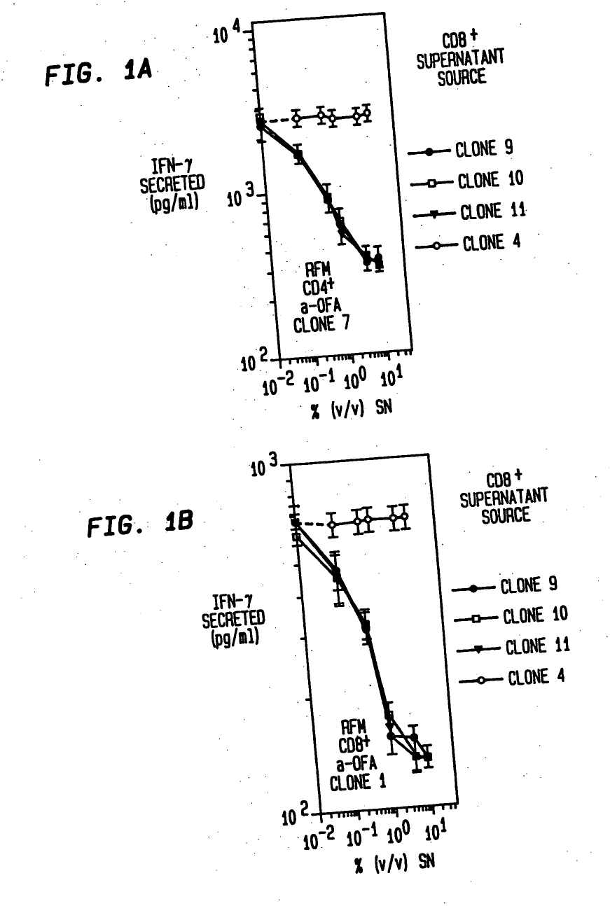

Supernatants from non-cytotoxic, CD8 T cell clones derived from long-term survivors of radiation carcinogenesis inhibit anti-oncofetal antigen cytotoxic T cell clone killing of syngeneic, oncofetal antigen+5T lymphoma cells (Rohrer, J. W., et al., supra.). The culture supernatants of three of these non-cytotoxic CD8 clones was assayed for their ability to inhibit the secretion of interferon-γ by the anti-oncofetal antigen CD4 T cell clone 7 and the anti-oncofetal antigen CD8 cytotoxic T (T_{c}) clone 1 subsequent to their restimulation by irradiated 5T RFM lymphoma cells. The supernatant from oncofetal antigen-specific T_{c} cell clone 4 was used as a negative control. Incubation for 24 hours in IMDM containing as much as 10% (v/v) final concentration supernatant from cytotoxic clone 4 had no inhibitory activity on the ability of either CD4 clone 7 (FIG. 1A) or cytotoxic CD8 clone 1 (FIG. 1B) to secrete interferon-γ after a 48 hour stimulation culture with irradiated RFM spleen cells±irradiated RFM 5T lymphoma cells. The supernatants from the three non-cytotoxic CD8 T cell clones, however, inhibited gamma interferon secretion in a dose-dependent manner with a 50% inhibition at 0.35-0.4% supernatant concentration (FIGS. 1A and 1B).

### EXAMPLE 16

Inhibitor of Interferon-γ Secretion in the Supernatants of Anti-OFA, Noncytotoxic, CD8 T Cell Clones is an Antigen-Non-Specific Inhibitor

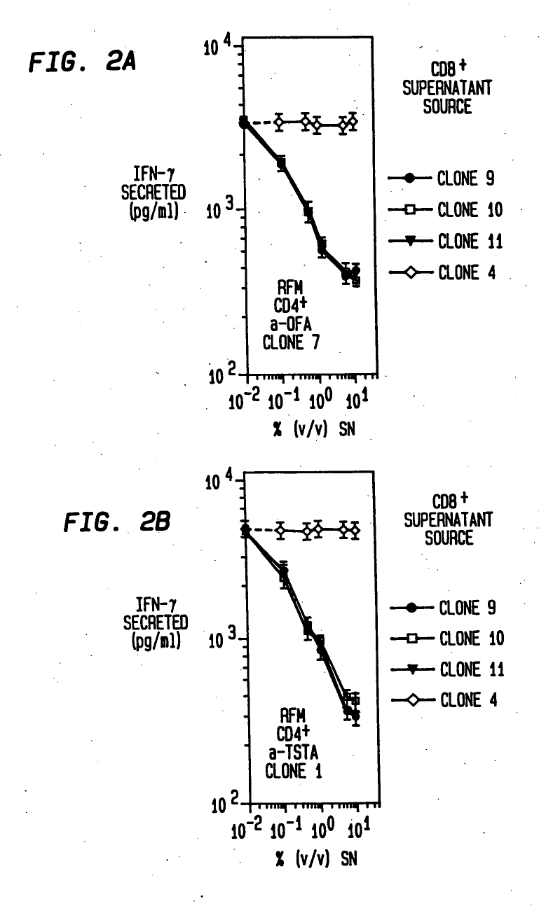

To determine if the inhibitor of interferon-γ secretion was an antigen-specific suppressor factor or not, the experiment described above was repeated except that two RFM tumor-reactive CD4 T cell clones as target cells were used. Clone 7 is oncofetal antigen-specific (Rohrer, J. W., et al., supra.) and clone 1 is specific for a 5T lymphoma TSTA [Rohrer, J. W., et al., J. Immunol. 152:754, (1994)]. As before, the cytotoxic clone supernatant had no inhibitory activity at any concentration, but all three supernatants from the oncofetal antigen specific, non-cytotoxic CD8 T cell clones inhibited both oncofetal antigen- and TSTA-specific T cell clone secretion of gamma interferon in a dose dependent manner (FIGS. 2A and 2B). Once again 50% inhibition was found at 0.35 to 0.40% (v/v) supernatant concentration. That both clones are inhibited suggests that the active factor is not oncofetal antigen-specific.

### EXAMPLE 17

Inhibitor of Interferon-γ Secretion in the Supernatants of Anti-OFA, Non-Cytotoxic, CD8 RFM T Cell Clones is not MHC-Restricted

To demonstrate that the inhibitor is not MHC-restricted, a RFM CD4 T cell clone 7 that recognizes an oncofetal antigen peptide:H-2^{f} class II protein complex (Rohrer, J. W., et al., supra.) and the BALB/c CD4 T cell clone 5 that recognizes an oncofetal antigen peptide:H-2d class II protein complex (Decker, et al, supra.) as the target cells for inhibition of gamma interferon secretion were utilized. If the inhibitor was MHC-restricted, it should only be able to inhibit the RFM clone.

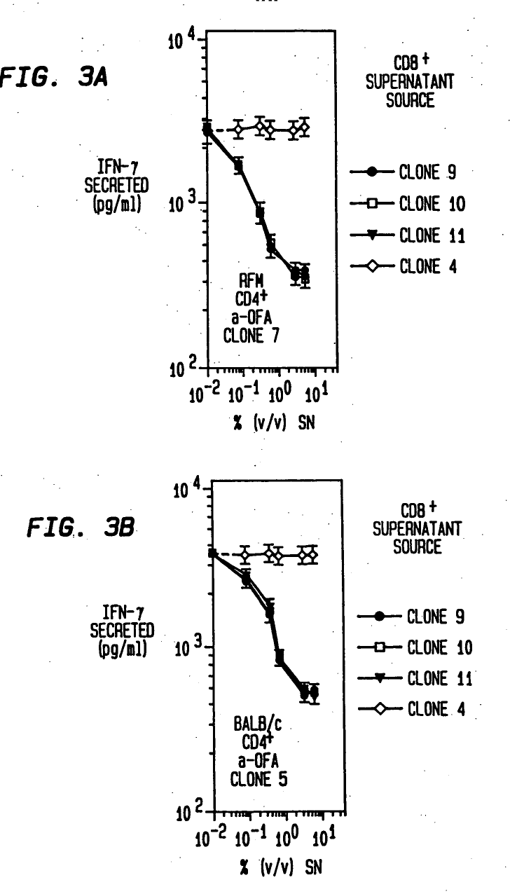

FIGS. 3A and 3B show that both RFM and BALB/c anti-oncofetal antigen clone interferon-γ secretion was inhibited in a dose-dependent manner by the culture supernatants of RFM non-cytotoxic T cell clones 9, 10, and 11. The inhibition was not the result of the presence of spent medium since no significant inhibition was seen if as much as 10% supernatant from RFM anti-oncofetal antigen, cytotoxic CD8 T cell clone 4 was used (p>0.95). However, 50% inhibition of both BALB/c and RFM target cell secretion of interferon-γ was obtained at 0.35 to 0.40% (v/v) supernatant concentration from the RFM anti-oncofetal antigen, non-cytotoxic CD8 T cell clones tested. None of the inhibitory supernatants were significantly different from any of the others (p>0.94), but each was significantly more inhibitory than that of cytotoxic clone 4 (p<0.01). Also, the dose response of the inhibitory supernatants was not significantly different on the RFM target clone than on the BALB/c target clone (p<0.02). Therefore, the inhibitory factor was neither antigen-specific nor MHC-restricted and so may be a cytokine.

### EXAMPLE 18

Supernatants from 5T Lymphoma Cell-Stimulated Non-Cytotoxic T Cell Clones Contain IL-10

Since IL-10 has been shown to be able to inhibit interferon-γ secretion by CD4⁺ T_{H}1 cells in mice [Fiorentino, et al., J. Exp. Med. 170:2081 (1989)] and since the inhibitor did not target cells by recognition of antigen:MHC expression, the supernatants of three different non-cytotoxic, anti-oncofetal antigen CD8 T cell clones and of three anti-oncofetal antigen T, cell clones for IL-10 secretion were assayed. FIG. 4 shows that while there was no IL-10 above the level of detection in the culture supernatants of 5T tumor cell-stimulated oncofetal antigen-specific, RFM T_{C} clones 2, 4, and 8 (Rohrer, J. W., et al., supra.), the supernatants from non-cytotoxic CD8 T cells had from 12.7-14.2 times more IL-10 than the T_{C} clone supernatants after stimulation with irradiated 5T lymphoma cells. This difference was significant at the p<0.0001 level. The inhibitory supernatants were used in 100 μl volumes in inhibition assays and had a 50% inhibition concentration of 0.35 to 0.40% (FIGS. 1-3). Since those supernatants had from 177 to 209 pg/ml, the 50% inhibition concentration for IL-10 in these assays is 6.2-8.4 pg/well, if IL-10 is the inhibitor.

### EXAMPLE 19

RFM 5T Lymphoma Cells are not the Source of IL-10 in 5T Cell-Restimulated Non-Cytotoxic CD8 T Cell Clone Cultures

After 24 hour restimulation with irradiated 5T cells, both unselected populations of cells and CD4⁻, CD8⁺ T cells produced from 208 to 255 pg/ml of IL-10 in a second culture in IMDM+IL-2. However, neither the selected CD4⁺, CD8⁺ T cells (tumor cells) nor phenotype selected or unselected cytotoxic T cell clones produced any detectable IL-10. Neither the selected nor unselected non-cytotoxic T cell clone cultures were significantly different from one another in the amount of IL-10 produce (p>0.9).

### EXAMPLE 20

Macrophages in the Restimulated Non-Cytotoxic T Cell Clone Cultures are not the Source of IL-10

Unselected, non-cytotoxic CD8 T cell clone cultures or cultures of CD4⁻, CD8⁺ T cell clone cultures produced 202-230 pg/ml of IL-10 in 24 hours after selection. The cytotoxic T cell clone cultures after restimulation did not produce detectable IL-10 and elimination of macrophages by anti-CD11b antibody+anti-rat IgG+complement did not affect the amount of IL-10 detected in cultures of either cytotoxic (p=1) or non-cytotoxic T cell clones (p>0.96) subsequent to restimulation.

### EXAMPLE 21

Anti-IL-10 Neutralizes the Non-Cytotoxic CD8 T Cell Clone Supernatant Inhibition of Interferon-γ Secretion

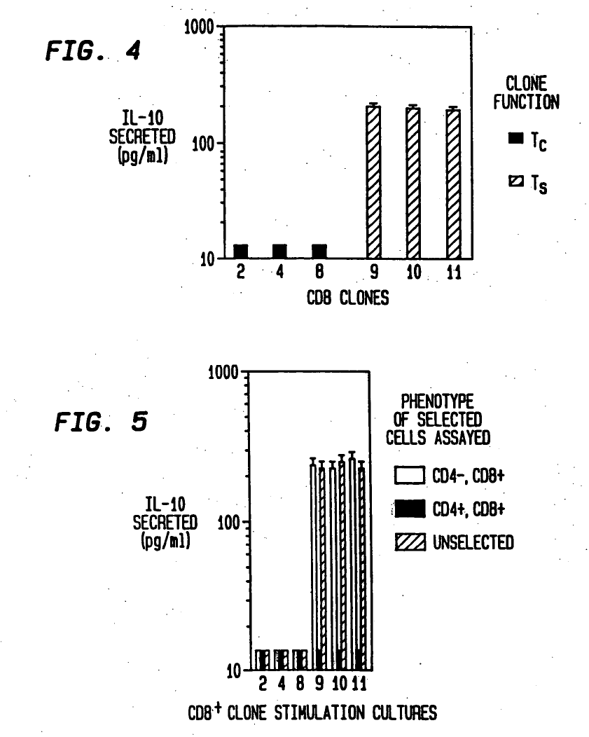

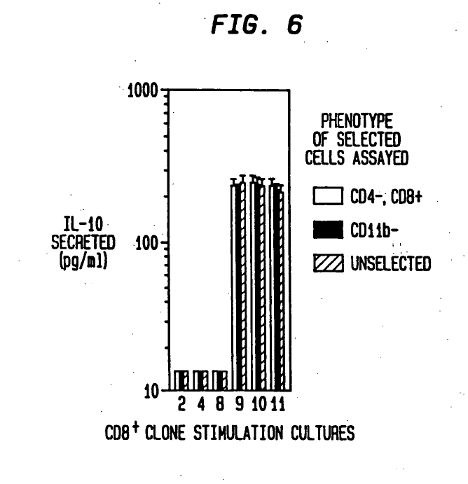

Monoclonal rat anti-mouse IL-10 IgM was titrated into the non-cytotoxic CD8 T cell clone supernatant:CD4 anti-oncofetal antigen T cell clone 4 incubation mixture to a final concentration varying from 1 to 25 μg/ml. As a control antibody, rat anti-mouse B220 IgM was titrated in to the same concentrations. FIG. 5A shows that even as little as 1 μg/ml of anti-IL-10 significantly increases the amount of interferon-γ secreted by clone 4 after stimulation with 5T lymphoma cells (p<0.03). As the amount of anti-IL-10 increases, the restoration of IFN-γ secretion increases until normal levels are reached by 25 [g/ml. Addition of this antibody had no effect on the T_{C} clone 4 supernatant treated CD4 clone secretion of IFN-γ (p>0.8). FIG. 5B shows that the presence of identical amounts of an irrelevant rat IgM monoclonal antibody does not block the non-cytotoxic CD8 T cell clone supernatant inhibition of anti-oncofetal antigen CD4 clone gamma interferon secretion (p>0.9).

### EXAMPLE 22

Anti-IL-10 Antibody Neutralizes the Non-Cytotoxic CD8 T Cell Clone Supernatant Inhibition of OFA-Specific CD8 Cytotoxic T Cell Activity

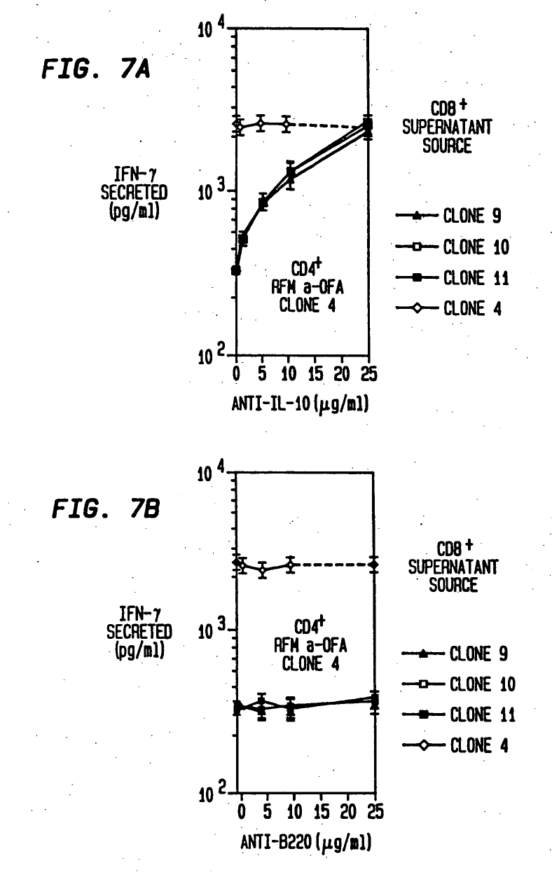

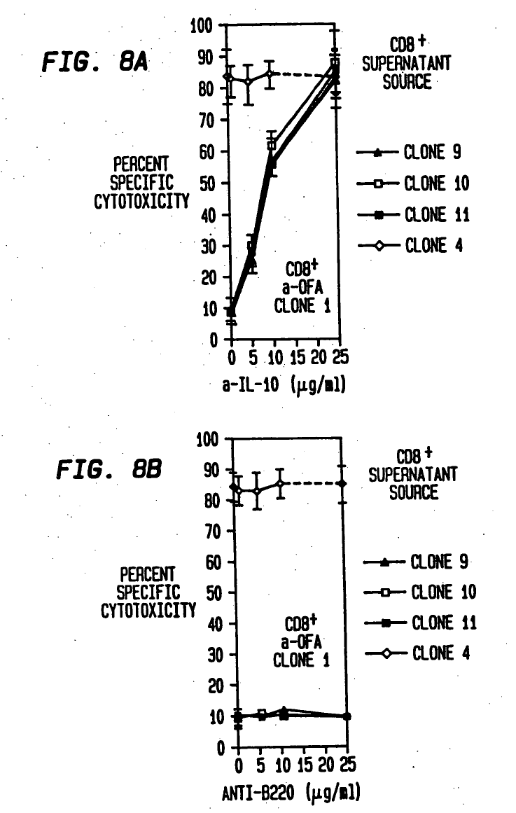

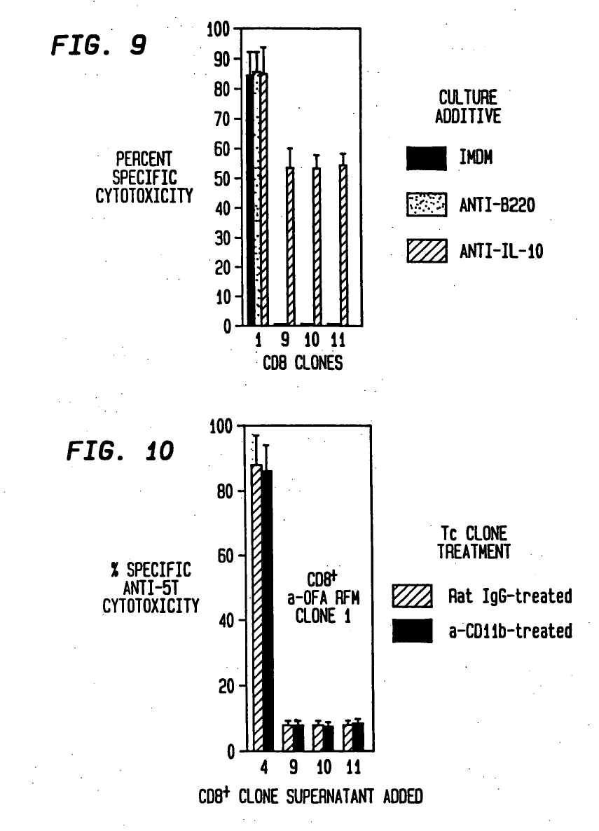

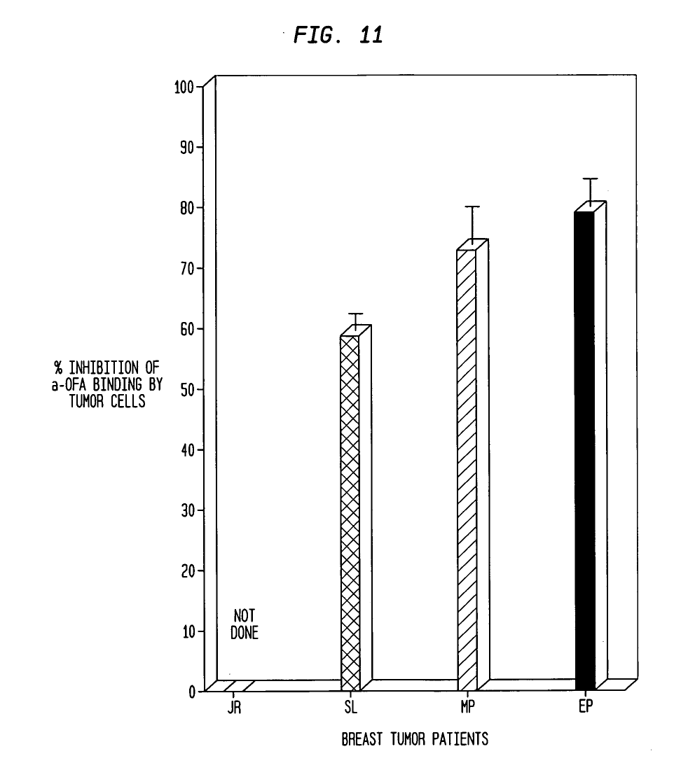

Since the supernatants from the non-cytotoxic CD8 T cell clones could inhibit tumor cell killing by oncofetal antigen-specific T_{C} clone cells (Rohrer, J. W., et al., supra.), the ability of anti-IL-10 monoclonal antibody to block inhibition by those supernatants of T_{C} clone 1 killing of RFM 5T lymphoma cells was determined. As in the experiment above, the anti-IL-10 or anti-B220 antibodies were titrated into the 24 hour incubation of the target clone with 5% supernatant from non-cytotoxic CD8 clones 9, 10, or 11 or the same amount of supernatant from T_{C} clone 4. FIG. 6A shows that as little as 5 μg/ml of anti-IL-10 antibody can significantly restore the cytotoxic activity of the anti-oncofetal antigen T_{C} clone (p<0.02). As the dose of anti-IL-10 antibody increases, so does the amount of specific cytotoxicity obtained with maximal activity restored at 25 μg/ml anti-IL-10 (p=0.001). FIG. 6B shows that the isotype control anti-B220 antibody does not significantly restore the cytotoxic activity of the T_{C} clone at any concentration used (p>0.9). Neither antibody affected the anti-5T cytotoxicity of T_{C} clone 1 cells which had been pre-treated with T_{C} clone 4 supernatant (which lacks IL-10 and is non-inhibitory) (p>0.8).

### EXAMPLE 23

Anti-IL-10 Antibody Restores Anti-5T Cytotoxic Activity to Oncofetal Antigen-Specific, Non-Cytotoxic CD8T Cell Clones

Because IL-10 is in the culture supernatants of 5T lymphoma cell-stimulated non-cytotoxic CD8 T cell clones and anti-IL-10 blocks the inhibitory activity of those culture supernatants, that the oncofetal antigen-specific, non-cytotoxic CD8 T cell clones might themselves be inhibited from killing 5T cells by the presence of their own IL-10 during activation was examined. Therefore, 10μg/ml anti-IL-10 or anti-B220 were added to the cytotoxicity assay containing noncytotoxic T cell clones 9, 10, and 11 plus irradiated, T cell-depleted RFM spleen cells+irradiated 5T lymphoma cells plus IL-2. For a positive control, the anti-oncofetal antigen T_{C} clone 1 was used.

FIG. 7 shows that in the presence of an anti-IL-10 antibody, all of the “non-cytotoxic” CD8 clones had significant cytotoxic activity against 5T cells (p<0.002). These clones, however, did not kill normal RFM spleen cells (data not shown). The amount of cytotoxicity is similar to that exhibited by clone 1 cells that had been pre-treated with the inhibitory supernatant in the presence of 10μg/ml of anti-IL-10 (FIG. 5A). No cytotoxic activity was restored by addition of anti-B220, i.e., the effect is specific. Thus, the inhibitory clones can function as effectors if the suppression induced by the IL-10 secreted by the clones is neutralized.

### EXAMPLE 24

Harvest of Human Mononuclear Cells (Lymphocytes and Monocytes)

Purification of human peripheral blood mononuclear leucocytes (lymphocytes and monocytes) is performed using a modification of the method of Boyum [Boyum, A., Nature 204:793 (1964)]. The modification involves the use of sterile Ficoll sodium diatrizoate solution of the proper density, viscosity, and which is isotonic with human leucocytes (Ficoll-Paque Plus) instead of just Ficoll. This modification has been shown to be an easy one-step, rapid, reproducible method for the preparation of viable lymphocytes in high yield from peripheral blood [Harris and Ukaejiofo, Brit. J. Haematol. 18:229 (1970); Ting and Morris, Vox Sang., 20:561 (1971); Fotino, et al., Ann. Clin. Lab. Sci. 1:131 (1971); Bain and Pshyk, Transplantation Proc. 4:163 (1972); Wybran, et al., J. Immunol. 110:1157 (1973); Fotino, et al., Vox Sang 21:469 (1971)].

a. Heparinized human blood is diluted 1:2 with RPMI-1640 tissue culture medium supplemented with 2mM L-glutamine, 100 units/ml of Penicillin G and 100 μg/ml of Streptomycin.

b. The diluted blood is layered in 4 ml aliquots onto a 3 ml layer of Ficoll-Paque Plus in sterile 15 ml conical centrifuge tubes with an internal diameter of 1.3 cm. This layering is done so that minimal mixing of the blood and the Ficoll-Paque Plus occurs.

c. The tubes containing the Ficoll-Paque Plus and the blood are centrifuged at 400×g for 30 minutes at 18-20° C.

d. At the end of this centrifugation, the mononuclear leucocytes are located in a band between the plasma and the Ficoll-Paque Plus and the erythrocytes and granulocytes are in a pellet at the bottom of the tube.

e. The plasma is pipetted off and the mononuclear cell layer from each tube is pipetted into a 50 ml centrifuge tube (all tubes' mononuclear cell layers combined into one tube) and 3 cell volumes of RPMI-1640 (as prepared in 1 a, above) is added to the 50 ml tube.

f. The mononuclear cell fraction tube is centrifuged at 60-100×g for 10 minutes at 18-20° C.

g. The supernatant is removed and the mononuclear cell pellet is resuspended in 10 ml of RPMI-1640 tissue culture medium supplemented as described in step 1a, above, and the cells transferredto a sterile 15 ml centrifuge tube and centrifuged at 60 100×g for 10 minutes at 18-20° C.

h. The supernatant is removed and discarded and cells resuspended in 1 ml of RPMI-1640 medium supplemented as in 1 a additionally containing 100 U/ml of recombinant human Interleukin-2, 10 U/ml of recombinant human Interferon-γ, and 10 units/ml of recombinant human Interleukin-6 and 10% (v/v) fetal calf serum (termed from here on complete RPMI-1640). IL-2 is utilized as a growth factor for T lymphocytes; -interferon is used to inhibit the outgrowth of Th2 helper T cells for antibody production [Gajewski and Fitch, J. Immunol. 140:4245 (1988)]; IL-6 is used to promote the outgrowth and function of T cytotoxic (TC) lymphocytes. Rogers, et al., J. Immunol. Methods 15:61 (1991). The cells are counted for viability using Trypan blue dye exclusion on a hemacytometer with a light microscope. Phillips, In: Tissue Culture: Methods and Applications. P. F. Kruse, Jr., ed. Academic Press, New York, pp. 406-408 (1973).

### EXAMPLE 25

Culture of Harvested Human Peripheral Blood Mononuclear Cells

a. The harvested, counted human blood mononuclear leucocytes (lymphocytes and monocytes) were cultured in complete RPMI-1640 (as defined in 1 h above) after addition of 3000 rad-irradiated autologous tumor cells. The cultures were set up in appropriate volumes such that there were 5×10⁵ viable blood mononuclear leucocytes/ml of culture and 5×10⁵ viable irradiated autologous tumor cells/ml of culture.

b. During this culture all T lymphocytes capable of responding to antigens expressed by the tumor cells become activated and begin to proliferate while non-responding lymphocytes and all monocytes begin to die. Thus, every 2-3 days, cell viability counts were done and culture volume adjusted to allow a viable cell density permitting continued growth and viability of the responding cells. After about 1 week, the responding cells constitute the majority of remaining cells and culture volumes were expanded to keep the cell density from outgrowing the nutrients and growth factors present.

c. Every two weeks the tumor-reactive lymphocytes must be restimulated with irradiated autologous tumor cells in the presence of autologous irradiated peripheral blood mononuclear leucocytes to keep IL-2 growth factor receptors expressed so they can continue to proliferate.

d. After the initial two weeks of culture subsequent to purification of the mononuclear cells from human blood, the residual living cells were counted and cloned by limiting dilution at 0.2 cell/well in 96 well plates. Rohrer, J. W., et al., J. Immunol. 152:754 (1994). Each well contained 10⁵ viable-irradiated autologous tumor cells (as the source of antigen) and 10⁵ viable-irradiated autologous peripheral blood mononuclear leucocytes to serve as antigen-presenting cells. After two weeks, those wells with one colony per well were harvested and expanded in the presence of irradiated autologous tumor cells and irradiated autologous peripheral blood mononuclear cells in complete RPMI-1640 medium.

e. After the clones were expanded and stabilized in their growth, they were cultured in RPMI-1640 which has all the supplements of complete RPMI-1640 except for -interferon and IL-6.

f. All cultures were done at 37° C. in a 95% air/5% CO₂ humidified atmosphere.

### EXAMPLE 26

Determination of T Cell Clone Specificity by Proliferation in Response to Oncofetal Antigen Protein (OFA)

a. Two days before clones were to be restimulated with autologous tumor cells, some of the culture was harvested, washed, and a viability count done. 2-10,000 viable tumor-reactive clone cells were then seeded into each well of 96 well culture plates along with 5×10⁵ viable 3000 rad-irradiated autologous peripheral blood mononuclear leucocytes plus various doses (15-300 ng/well) of purified OFA or non tumor cell membrane proteins on nitrocellulose particles prepared using the method of Strandring and Williams [Standring and Williams, Biochem. Biophys. Acta 508:85 (1978)] and Abou-Zeid et al. [Abou-Zeid, et al., J. Immunol. Methods 98:5 (1987)] as described previously (Rohrer, J. W., et al, supra.). The cells are cultured in complete RPMI-1640 medium.

b. The cultures are incubated for 48 hours at 37° C. in a humidified 95% air/5% CO₂ atmosphere.

c. The cultures are then pulsed with 10 μM 5-bromodeoxyuridine (100 μl/well) and cultured for another 24 hours under the conditions described in 3b.

d. At the end of that incubation, the plates are centrifuged at 300×g for 10 minutes at 4° C. to pellet the cells. The supernatant is then removed by tapping onto absorbent paper and the plates dried for 60 minutes at 60° C. After the hour of drying, the cells are fixed in 70% ethanol (200 μl/well) for 30 minutes at room temperature.

e. At the end of that incubation, the supernatant is removed by tapping onto absorbent paper and the protein-binding areas of the plate blocked by a 30 minute room temperature incubation with 200 μl of 1% (w/v) nonfat dry milk protein in 50 mM Tris-HCl; 150 mM NaCl, pH 7.4.

f. After that incubation, the blocking buffer is removed by tapping the plates onto absorbent paper. Each well then receives 100 μl of 1:100 diluted anti-bromodeoxyuridine antibody which is conjugated with horseradish peroxidase and will bind to the DNA into which bromodeoxyuridine was incorporated during the S phase of the cell cycle of proliferating cells. This is incubated 90 minutes at room temperature.

g. After this incubation, the antibody solution is removed by tapping on absorbent paper and then the wells are rinsed 3 times with 200 μl of 0.1 M phosphate-buffered saline, pH 7.4, being careful not to disturb the cells on the bottom of the wells. Excess fluid is removed by tapping on absorbent paper.

h. 100 μl of room temperature-equilibrated substrate solution 3,3′5,5′-tetramethylbenzidine (TMB) in 15% (v/v) DMSO is added to each well. The plate is covered and mixed at room temperature until color development is sufficient for optical density measurement (5-3 0 minutes). When the required color intensity is achieved, the reaction is stopped by adding 25 μl of 1 M sulphuric acid to each well.

i. The optical density is read in a microELISA plate reader at 450 nm within 5 minutes. This assay is as sensitive as using [3H]-thymidine incorporation to measure proliferation [Porstmann, T., et al., J. Immunol. Methods 82:169 (1985)], but has the advantage of not dealing with radioactive material.

### EXAMPLE 27

Determination of T Cell Subclass and Cytokine Produced by Cloned or Peripheral Blood T Lymphocytes

To determine the subclass of OFA-specific T lymphocyte clones, whole peripheral blood T lymphocytes, or tumor reactive peripheral blood lymphocytes, flow cytometry is used while measuring CD4, CD8, and αβ or δγ T cell receptor expression.

a. First, the cells being observed are divided into two sets and stained for three color analysis. Both sets are stained with FITC conjugated monoclonal anti-human CD4 and R-PE-conjugated monoclonal anti-CD8 and one is also stained with Cy-chrome conjugated monoclonal anti-human αβ TCR antibody while the other set is stained with Cy-chrome anti-human δγ TCR antibody. All three fluorochromes are excited by the 488 nm laser line, but will emit at 520 nm (FITC), 576 nm (R-PE), and 670 nm (Cy-Chrome).

b. To inhibit non-specific staining, a 10-fold excess of irrelevant monoclonal mouse antibodies of the same isotype is included in the buffer along with the three fluorochrome-conjugated antibodies. Also the buffer (Dulbecco's PBS, pH 7.2) contains 0.1% (v/v) sodium azide to block shedding of antigen.

c. The concentration of the antibodies needed to give optimal specific staining is determined experimentally whenever a new lot of antibody is obtained. All of these antibodies are obtained from Pharmingen, Inc.

d. These data indicate which clones are CD4+, which clones are CD8+, and which type of TCR each clone uses.

e. The same experimental methods are used with uncloned peripheral blood T lymphocytes freshly purified from cancer patients or normal controls or after being stimulated once with autologous tumor and then waiting for the tumor-reactive T lymphocytes to expand in culture.

f. These studies on freshly isolated mononuclear leucocytes determine if the cancer causes an overall change in CD4⁺ or CD8⁺T cell frequencies or in the frequency of each, which uses a given type of T cell antigen receptor.

g. The experiments with expanded, but not cloned tumor-reactive T lymphocytes determine if any change is induced by the cancer in the frequency of these T cell subsets which can recognize tumor-expressed antigens.

To determine more clearly what functional activity these T cells have, three color analysis is utilized, but intracellular interferon-γ, intracellular IL-10, and surface CD4 or CD8 is observed. Interferon gamma is made and secreted by Th1 helper cells for cell-mediated immunity and by CD8 cytotoxic T lymphocytes. IL-10 is a cytokine which inhibits cell-mediated immunity and gamma interferon secretion especially and it has been found to be made by OFA-specific CD8 T cell clones which are not cytotoxic, but through IL-10 can inhibit anti-tumor cytotoxic T cell function. Rohrer, J. W., et al., J. Immunol. 154:2266 (1995); Rohrer and Coggin, J. Immunol. 155:5719 (1995).

h. The clones and tumor-reactive, uncloned peripheral blood mononuclear cell cultures from cancer patients is cultured with 3 μM monensin for 4-6 hours before cell harvest to block intracellular transport of proteins and thus have an accumulation of cytokines in the Golgi apparatus of the cells.

i. Freshly harvested and purified peripheral blood mononuclear leucocytes is placed in culture for 2 hours in complete RPMI-1640 supplemented as complete RPMI-1640 (as described in section 1h) except that no gamma interferon or IL-6 is present. After the 2 hours at 37° C. in a humidified 95% air/5% CO₂ atmosphere, monensin is added to 3 μM final concentration and the cells continued in culture for 4-6 hours.

j. In order to block nonspecific staining via Fc receptor binding, all cells are incubated with a 10-fold excess of irrelevant mouse monoclonal antibody of the same isotype as the fluorochrome conjugated antibodies for 5 minutes before and continually during staining of the cells.

k. The cells are divided into two groups and stained with the experimentally determined optimal amount of FITC-conjugated monoclonal anti-human CD4 CD8 to determine which clones are making IL-10 (and are, thus, probably inhibitory, non-cytotoxic T cells) as well as determining which and how many CD4 clones are making either or both of these cytokines.

l. Using this technique with freshly harvested mononuclear leucocytes from cancer and normal patients, it is determined if there is an overall effect of the cancer on certain cytokine-producing T cell populations.

m. Using this technique with tumor-reactive, but uncloned peripheral blood T cells from cancer patients demonstrates whether the cancer has an effect on certain cytokine-producing tumor-reactive T cell populations.

n. While it has been shown that the amount of fluorescence detected for most intracellular cytokines is proportional to the amount found secreted by those same cells in culture supernatants, [Elson, et al., J. Immunol. 154:4294 (1995); Jung, et al., J. Immunol. Methods 159:197 (1993)] that is not the case for interferon. Elson, et al., supra.; Vikingson, et al., J. Immunol. Methods 173:219 (1994). Thus, culture supernatants are taken 48 hours and 96 hours after restimulation of clones and of uncloned, tumor-reactive cancer patient peripheral blood T cells and assay by ELISA for interferon-as described previously. Rohrer, J. W. et al., supra.

### EXAMPLE 28

Human Breast Carcinoma Patients Develop Clonable Oncofetal Antigen-Specific Effector And Regulatory T Lymphocytes 1

In this example, breast carcinoma patients' peripheral blood mononuclear leucocytes were stimulated in vitro with autologous tumor cells in the presence of IL-2, γ-IFN, and IL-6 for 2 weeks, to determine if 44 kD OFA is also immunogenic for human T lymphocytes. The tumor-reactive cells were then restimulated and cloned by limiting dilution and the clones analyzed. We established 24, 19, 11, and 16 tumor-reactive clones from the 4 respective patients. Of those 4, 6, 4, and 7, respectively, proliferated specifically to purified OFA. Both CD4 and CD8 OFA-specific clones were established which responded equally well to purified OFA or iLRP. All were CD3+, αβ TCR+. All CD4 clones secreted γ-IFN, but neither IL-4 nor IL-10. Both γ-IFN-secreting cytotoxic CD8 clones and IL-10-secreting inhibitory CD8 clones were established. Thus, during human cancer development, the same types of OFA-specific effector and regulatory T cells are induced as during murine T lymphomagenesis.

Thus, it was found that CD4+ TH1, CD8+ TC and IL-10-secreting, non-cytotoxic CD8+ T cells which proliferated specifically in response to purified OFA were clonable from all 4 breast carcinoma patients selected as they became available for biopsy. While the number of tumor-reactive clones able to be established and the profile of the OFA-specific clones varied among the patients, all had all three subclasses of T cell clones.

It was also found that recombinant immature laminin receptor protein (30) stimulated the OFA-specific clones' proliferation in a dose response identical with that shown using purified OFA. This matched exactly similar experimental results using iLRP to stimulate murine 44 kD OFA-specific T cell clones established from survivors of X-irradiation-induced lymphomagenesis (27). This strengthens the identification of OFA as immature laminin receptor protein which has been suggested by peptide sequences and monoclonal antibody reactivity. Thus, it appears that OFA, not only is detectable on human tumor cells, but also induces the same type of T lymphocyte immune responses during tumor development in man as it does in experimental animals.

Patients

The four women were 30-45 years old, had invasive ductal carcinoma of the breast, and tumor tissue and initial blood was taken at the time of mastectomy. Subsequent blood samples were obtained every two weeks by venipuncture for 8 more weeks.

Peripheral Blood Mononuclear Cell Purification

Peripheral blood mononuclear cells were purified from the breast carcinoma patients' blood using a modified method of Boyum (31). Briefly, the heparinized blood is diluted 1:2 in sterile RPMI-1640 medium and 4 ml aliquots is layered over 3 ml aliquots of Ficoll-Paque Plus (Pharmacia Biotech, Piscataway, N.J.) in 15 ml tissue culture treated sterile polystyrene centrifuge tubes (Sarstedt, Newton, N.C.) and then centrifuged for 35 min. at 400×g at 20 C. Following pipetting off of the upper plasma, the lymphocyte layer at the interface is removed from each tube, pooled and diluted 1:3 in RPMI-1640 medium, mixed gently to resuspend the cells and centrifuged at 100×g for 10 min. at 20 C. After removing the supernatant, the pellet is resuspended in 10 ml medium and centrifuged at 100×g for 10 minutes at 20 C. The pellet is then resuspended in sterile RPMI-1640 medium and counted for viability using Trypan blue dye exclusion.

Preparation of Autologous Human Breast Carcinoma Cell

Breast tumor tissue in excess of that needed for pathologic diagnosis was obtained from the surgical Pathology Laboratory at the University of South Alabama-Knollwood Hospital and used for this study. The tissue was minced into very small pieces, which were then passed through sieves of gradually reduced mesh by applying gentle pressure with the piston of a disposable plastic syringe and the cells were washed through the sieve in sterile RPMI 1640 medium containing 10% fetal calf serum, 100 U/ml penicillin and 100/μg/ml streptomycin. The cell suspension was then passed through a nylon mesh centrifuged at 2000 rpm for 10 min and the sedimented cells used for T cell stimulation. Such cell preparations were 65-75% viable. Tumor cells not used for in vitro stimulation of T cells at that time were cryopreserved in sterile RPMI 1640 with 10% (v/v) dimethyl sulfoxide and 50% (v/v) fetal calf serum, in a freezer at −70° C. When needed for restimulation, tumor cells were thawed rapidly, diluted in an excess of sterile RPMI 1640 plus 10% (v/v) FCS and washed twice.

Cell Lines

The anti-OFA IgM-producing hybridoma 115 (24) is carried as an ascites tumor from which ascites fluid is collected and mAb 115 purified as described previously (26).

Anti-T Cell mAbs

Monoclonal mouse anti-human CD4 IgG1 from hybridoma 34930.111 was obtained from R&D Systems, Inc. (Minneapolis, Minn.). Monoclonal mouse anti-human CD8 IgG1 from hybridoma RPA-T8, monoclonal mouse anti-human CD3 IgG1 from hybridoma UCHT1, and monoclonal mouse anti-human TCR IgM from hybridoma V 5T-TCR.01 were obtained from Pharmingen (San Diego, Calif.).

T Cell Clone Production

Peripheral blood mononuclear cells isolated as described above from breast carcinoma patient heparinized blood were cultured with irradiated autologous breast carcinoma cells in sterile RPMI-1640 medium containing 2 mM L-glutamine, 100 U/ml Penicillin G, 100 μg/ml streptomycin sulfate, and 10% CPSR-3 serum replacement supplement (complete RPMI 1640) (Sigma Chemical, St. Louis, Mo.). The cultures contained 100 U/ml of recombinant human IL-2, 10 U/ml of recombinant human γ-interferon, and 10 U/ml of recombinant human IL-6 (R&D Systems, Minneapolis, Minn.). We utilized IL-2 as a growth factor for T cells, γ-IFN to inhibit outgrowth of TH₂ helper T cells for Ab production (32), and IL-6 to promote outgrowth and function of T_{C} cells (33). After two weeks of culture, the reactive cells (that is, T cells which proliferated during initial culture of breast carcinoma patient peripheral blood mononuclear leucocytes (PBML) with irradiated autologous breast carcinoma cells in the presence of the cytokine-supplemented RPMI-1640 medium) were restimulated with irradiated autologous tumor cells+irradiated autologous peripheral blood mononuclear cells in complete RPMI-1640 medium containing IL-2, IL-6, and γ-interferon as described above and cloned by limiting dilution (1 tumor-reactive T cell/5 wells) using essentially the same technique previously published (27) except that the tumor stimulus was the autologous breast carcinoma cells and antigen presenting cells were from the irradiated peripheral blood mononuclear cells (autologous). After growth of the clones had stabilized, only recombinant human IL-2 was added (no IL-6 or γ-IFN) at subsequent restimulations. These clones had to be restimulated with autologous tumor cells in the presence of cytokines and autologous peripheral blood mononuclear cells every two weeks to maintain viability and proliferation. During cultures set up to determine which cytokines were produced by the clones, the clones were restimulated in the presence of irradiated autologous tumor cells and irradiated peripheral blood mononuclear cells which had been depleted of T lymphocytes by negative selection on anti-CD3 mAb-coated Petri plates using the method of Wysocki and Sato (34) except that anti-CD3 Ab was used and was added to the plates on the day of the cell separation.

Determination of T Cell Clone Specificity by Proliferation in Response to Antigen

At the time of the 2-week restimulation of the clones to maintain their proliferation, the cloned cells were harvested, washed in complete RPMI-1640, and a viability count done. A portion of the cells was saved to be used in the proliferation assay. The proliferation assay was done with 10,000 viable cloned cells/well+irradiated autologous peripheral blood mononuclear cells+various doses of purified 44 kD OFA protein from a murine thymic lymphoma cell line or purified 44 kD protein from normal murine thymus or recombinant immature laminin receptor protein (iLRP) on nitrocellulose particles or an equivalent amount of unconjugated nitrocellulose particles in 96 well plates. After 24 hours, 10 μl of 5-bromodeoxyuridine (BUDR) is added to each well to a final concentration of 10 μM BUDR/well. The cells are continued to be incubated for another 24 hours. Proliferation is assayed using the Biotrak bromodeoxyuridine incorporation assay (Amersham, Arlington Heights, Ill.). At the end of that incubation, the plates are centrifuged at 300×g for 10 minutes and the labeling medium removed. The cells are dried for 1 hour at 60 C. The cells are then fixed with an ethanol fixative for 30 minutes at room temperature, the fixative removed, the wells coated with blocking buffer (1% protein in 50 mM Tris-HCl; 150 mM NaCl, pH 7.4) and incubated for 30 minutes at room temperature. The blocking buffer is removed and 100 μl of 1:100 diluted peroxidase-labeled anti-BUdR added to each well and the plates incubated for 90 minutes at room temperature. The antibody solution is then removed and the wells washed three times with 300 μl/well of wash buffer. 200 μl of 3,3′,5,5′-tetramethylbenzidine (TMB) in 15% (v/v) DMSO is added to each well and the plate covered and incubated for 5-30 minutes at room temperature. When the required color density is reached, the reaction is stopped by adding 25 μl of 1M sulphuric acid to each well and the plate read on a microELISA reader at 450 nm.

Determination of T Cell Clone Surface Ag Phenotype by mAb and Complement Depletion

One week after antigen restimulation, part of each T cell clone culture was harvested, washed three times by centrifugation in complete RPMI-1640 medium, and a viability count was done. The cells were diluted to 1×10⁶ viable cells/ml and their surface antigen phenotype was determined by cytotoxicity with mAbs+facilitating antiserum+complement, as previously described (27). The counted cells were pelleted and resuspended in 1 ml of anti-CD4, anti-CD8, anti-CD3, or anti-αβTCR Ab diluted optimally in complete RPMI-1640 medium. For all Abs used, the optimal dilution was 1:15. Control Ab was normal mouse IgG. After Ab and complement treatment, cells were pelleted by centrifugation, washed three times in complete RPMI-1640 medium and resuspended in 1 ml complete RPMI-1640. A viability count was done by Trypan blue dye exclusion. The percentage of cells specifically killed or lysed by the experimental antibody and complement treatment was calculated by knowing the number of total and viable cells in each tube at the beginning and comparing the nonspecific killing effect of the control Ab+facilitating antiserum+complement with the killing by the experimental Abs+facilitating Ab+complement.

ELISA of IFN-γ, IL-4, and IL-10 Production by T Cell Clones

Quantikine assay kits for IFN-γ, IL-4, and IL-10 from R&D Systems (Minneapolis, Minn.) were used. They utilize horseradish peroxidase labeled anti-cytokine antibody to detect cytokine that is captured on the anti-cytokine-coated plates. TMB is the substrate that is added and the color reaction is stopped with 2 N sulphuric acid and color read at 450 nm. The γ-interferon standard curve was linear between 5 pg/ml and 500 pg/ml and the minimum amount detectable in this assay was 3 μg/ml. The IL-4 standard curve was linear between 8 pg/ml and 2000 pg/ml with the minimum amount detectable being 4.1 pg/ml. The IL-10 standard curve was linear between 5 pg/ml and 500 pg/ml. The minimum amount of IL-10 detectable was 3 pg/ml.

Determination of T Cell Clones' Cytotoxic T Cell Activity Against Autologous Breast Carcinoma Target Cells

Cytotoxicity assays were performed using the CytoTox96 nonradioactive cytotoxicity assay kit produced by Promega (Fisher Scientific, Norcross, Ga.). The assay quantitatively measures lactate dehydrogenase (LDH), a stable cytosolic enzyme that is released upon cell lysis. Released LDH in culture supernatants is measured with a 30 min. coupled enzymatic assay resulting in the conversion of a tetrazolium salt to a red formazan product (35). The amount of color formed is proportional to the number of lysed cells. Color was quantitated using a Titertek Multiskan MC ELISA reader (Fisher Scientific, Norcross, Ga.) which measured absorbance at 492 nm. The setup of the assay was the same as previously described for testing mouse clone cytotoxicity against mouse tumors (27) except that the medium used was RPMI-1640 and autologous breast carcinoma cells were used as targets. The percent specific cytotoxicity was calculated using the formula listed below:

$$\% Cytotoxicity = \frac{( Experimental - Effector Spontaneous ) - Target Spontaneous}{Target Maximum - Target Spontaneous}$$

There is much less of a spontaneous release of LDH in this assay than of 51 Cr in a traditional 51 Cr release cytotoxicity assay, and therefore higher specific cytotoxicity percentages are achieved. Determination of the ability of anti-IL-10 to convert noncytotoxic CD8, OFA-specific T cell clones to cytotoxic clones. To determine whether the clones from patient MP and EP that were secreting IL-10 were being inhibited by it, the cells were harvested one day before the 2 week restimulation culture and set up in with 10 μg/ml mouse monoclonal anti-human IL-10 IgG1 (from hybridoma MAB217; R&D Systems, Inc, Minneapolis, Minn.) or normal mouse IgG for 24 hours as described previously (27). The cells were then harvested, washed three times with complete RPMI-1640 medium and viability counts done. The cells were then added to a 4 hour cytotoxicity assay against autologous and allogeneic breast carcinoma cells as described above and previously (27) except that anti-IL-10 or control IgG was added to a final concentration of 10 μg/ml.

ELISA Assay for OFA/iLRP on Breast Carcinoma Cells

Flat-bottomed 96-well plates [Nunc-Immunoplate 1 (Vanguard International) were coated with riLRP 300 ng/100 μl/well and post coated with 1% Bovine Serum Albumin (BSA) in PBS, pH 7.2. A direct binding curve for the anti-OFA mAb115 was first generated by incubating 100 μl of a serial dilution of the antibody in 0.5% BSA in PBS at 37° C. for 1 hr with the riLRP. The plate was washed four times for 5 min each with PBS-T solution (PBS containing 0.5% Tween-20). The plate was further incubated with a biotinylated goat anti-mouse μ-chain specific antibody at 1:5000 dilution in 0.5% BSA in PBS for 1 hr. The plate was washed again as described previously and 100 μl of an AB reagent (avidin:biotinylated horseradish peroxidase, Vector Laboratories; one drop of each in 10 ml PBS-T) were added to each well of the microplate and incubated for 30 min at room temp. The plate was washed as described previously. Finally, 100 μl of the substrate solution (2,2′-azino-di-(3-ethylbenz-thiazoline sulfonate in 0.1 citrate puffer, pH 4) were added to each well. After an incubation period of 30 min, the colored product was measured spectrophotometrically at 410 nm in a microELISA reader. The tests were done in triplicate. For assaying for OFA/riLRP on breast carcinoma cells a competitive ELISA was performed using a dilution of the mAb 115 which was below the saturation point (about 70% of the plateau level) to obtain maximal sensitivity to inhibition. 5×104 breast carcinoma cells from each patient were incubated with 0.5 ml of the antibody dilution overnight at 10° C. The cells were sedimented by centrifugation and 100 μl of the supernatant were applied to each of three wells on a microplate coated with riLRP and the ELISA reaction continued as described above. Although, we were unable to obtain sufficient normal breast tissue cells from the patients, all assays were run with OFA+MCA1315 fibrosarcoma cells as a positive control and normal BALB/c mouse spleen cells (OFA) as a negative control. The percent inhibition was calculated from the formula:[1-(Pre-absorption Ab OD410-background OD410)/Post-absorption Ab OD410-background OD410)]×100. Experimental values are presented as the mean±S.E.M. of the number of individual assays. This assay has been very reproducible since its development in this lab in 1985 (24). As previously published (24), only 2-9% absorption of the 115 IgM anti-OFA monoclonal antibody is seen with normal human tissues, while human tumors of various types absorb from 22-89% of the 115 IgM anti-OFA activity. Thus, approximately 10 times as much antibody is reproducibly absorbed by cancer cells as is by normal human tissue.

Recombinant iLRP

The cDNA encoding iLRP was cloned from a 7-day gestation embryonic library prepared from Swiss/Webster mouse. The coding region was cloned into an expression vector under control of the tac promoter, and the protein was expressed in E. coli. Inclusion bodies were isolated and solubilized in 6M guanidine hydrochloride in 20 mM Tris, pH 8.0, 0.1 M NaCl, 2 mM EDTA, 0.02% azide. The solubilized protein was added to six volumes of 20 mM Tris pH 8.0, 1 M guanidine HCl, 2 mM reduced glutathione, 0.2 mM oxidized glutathione, was renatured for 18 hours at 4C, and then dialyzed against 20 mM Tris pH 8.0, 0.1 M NaCl, 0.04% azide. Preparation and solubilization of 5T plasma membranes Membrane fractions from 5T lymphoma cells grown in culture or normal thymus cells were prepared using the method of Standring and Williams (36). Protease inhibitors aprotinin (100 KIU/ml), PMSF (1 μM), leupeptin (15 μM), N— -p-tosyl-L-lysine chloromethylketone (50 μM), and soybean trypsin inhibitor (5 μg/ml) were used to minimize membrane protein degradation.

Preparation of Ag-Bearing Nitrocellulose Particles

OFA was isolated from 5T lymphoma membrane extract by immunoaffinity chromatography on a 115 mAb-Sepharose column, as previously described (24). Eluted 5T membrane material or the normal thymus membrane preparation was mixed with the sample buffer (v/v) and subjected to 10-20% gradient SDS-PAGE according to the method of Laemmli (37). Separated proteins were transferred to nitrocellulose (38), made visible by staining with Ponceau S (39) and a nitrocellulose strip carrying the 44 kD bands was cut. Each nitrocellulose strip was processed to obtain Ag-bearing particles using the method of Abou-Zeid et al. (40).

Statistics

Where multiple experiments were performed on each tissue, the data was analyzed for significant differences using Student's t-test. The data from experiments in which dose response curves were generated were analyzed using analysis of variance. A p value of <0.05 was considered significant.

Results

The breast carcinoma patients' tumors expressed oncofetal antigen. To be sure that the lack of OFA-reactive T cell clones did not just reflect the lack of sensitization during tumor development, tumor tissue taken from each patient was assayed for its ability to absorb anti-OFA monoclonal antibody 115 reactivity to riLRP in an ELISA absorption assay (24). FIG. 11 shows that all patients' tumors that were tested absorbed 57%-78% of the anti-OFA/iLRP activity. Patient JR's tumor cells had been used up in other assays so could not be tested. Thus, the breast carcinomas from the 3 patients that were tested were expressing oncofetal antigen. We were unable to obtain sufficient normal breast tissue from the patients to use as direct negative controls. However, all assays were done not only with the human breast carcinoma tissue, but also, BALB/c mouse fibrosarcoma cells as positive controls and BALB/c mouse spleen cells as OFA negative normal tissue controls. This assay has been very reproducible over the past 13 years and normal human tissue always absorbs at least one-tenth as much antibody as does human cancer tissue (24).

Human Breast Carcinoma Patients' Peripheral Blood Contains Clonable CD4+ and CD8+ Tumor-Reactive T Lymphocytes

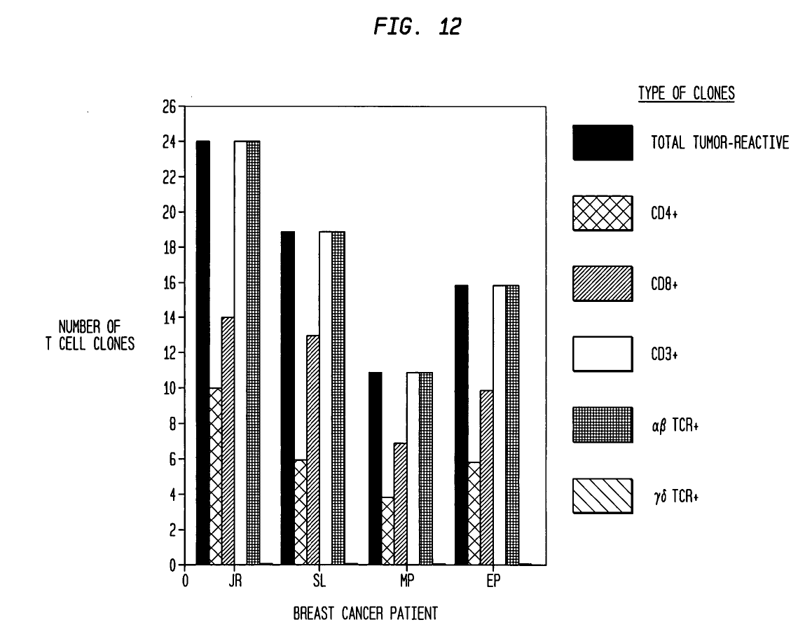

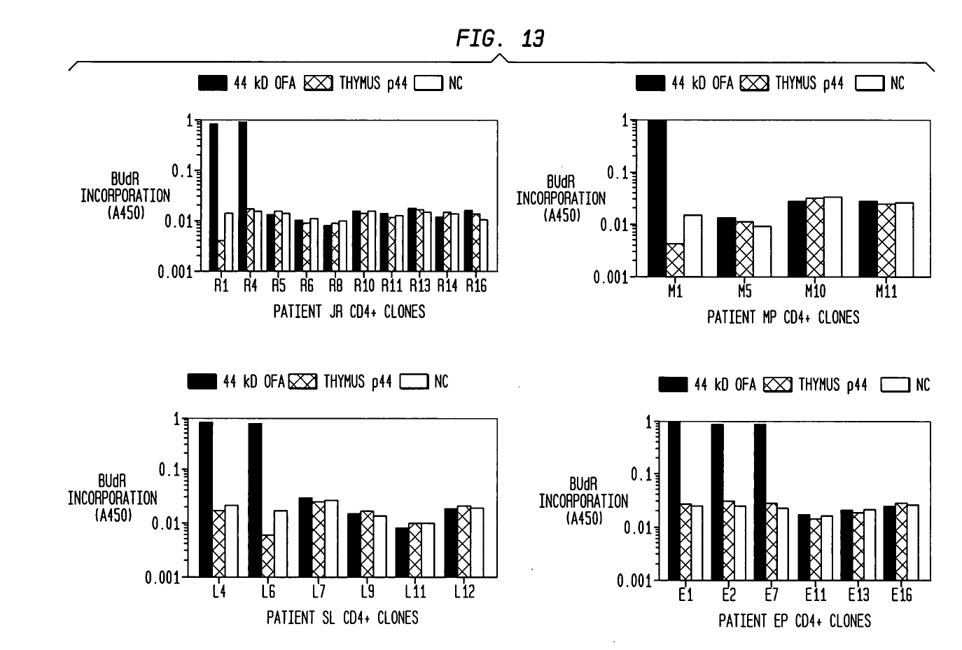

Culture of peripheral blood mononuclear leucocytes (PBML) with irradiated autologous breast carcinoma cells in the presence of recombinant human IL-2, γ-IFN, and IL-6 followed by restimulation with irradiated autologous tumor cells and limited dilution cloning, allowed establishment of 24, 19, 11, and 16 tumor-reactive T cell clones from the four patients respectively (FIG. 12). The term tumor-reactive is an operational definition in that these T cells grew out of cultures of breast carcinoma patient's PBML cultured with irradiated autologous breast carcinoma cells in the presence of the cytokines mentioned above and were restimulated by autologous breast carcinoma cells in the presence of irradiated autologous PBML and cytokines during cloning. Thus, presumably, the T cells which proliferated did so because of recognition of some epitope on the autologous tumor cells since subsequent culture with only irradiated autologous PBML and cytokines did not stimulate proliferation or cytokine production by the clones (FIGS. 13, 15, 16, and data not shown). All of the clones express CD3 and T cell antigen receptors. All clones were killed >87% with monoclonal anti-CD3 +facilitating anti-mouse IgG antibody+complement or monoclonal anti-human αβ TCR IgM+complement. Isotype control antibodies+facilitating antibody+complement killed <2.2% of the cells. Anti-αβ TCR IgM+complement similarly killed <3% of the cells. Of the CD3+, αβ TCR+ clones, 36.8 (±2.4)% were CD4+, CD8 T cell clones and 63.2 (μ 2.4)% were CD4, CD8+ T cell clones. However, analysis of uncloned, CD3+ cells (T lymphocytes) purified from the peripheral blood of the tumor patients showed 64.9±4.8% CD4+ cells and 35.1±4.8% CD8+ cells (data not shown). Thus, while the tumor-reactive T cells had an inverted frequency of CD4:CD8 T cells (0.58), the uncloned peripheral blood T cells showed a normal 1.85 CD4:CD8 ratio. This may reflect some artifact of cloning culture or may reflect a skewing toward CD8 T cells in the tumor-reactive portion of the total T cell population during breast carcinoma development. A similar CD8 predominance was also seen in clones derived from irradiation-induced T lymphoma developing RFM mice (27). Both CD4+, αβ TCR+ and CD8+, αβ TCR+ T cell clones that are OFA-reactive are established from breast carcinoma patient peripheral blood.

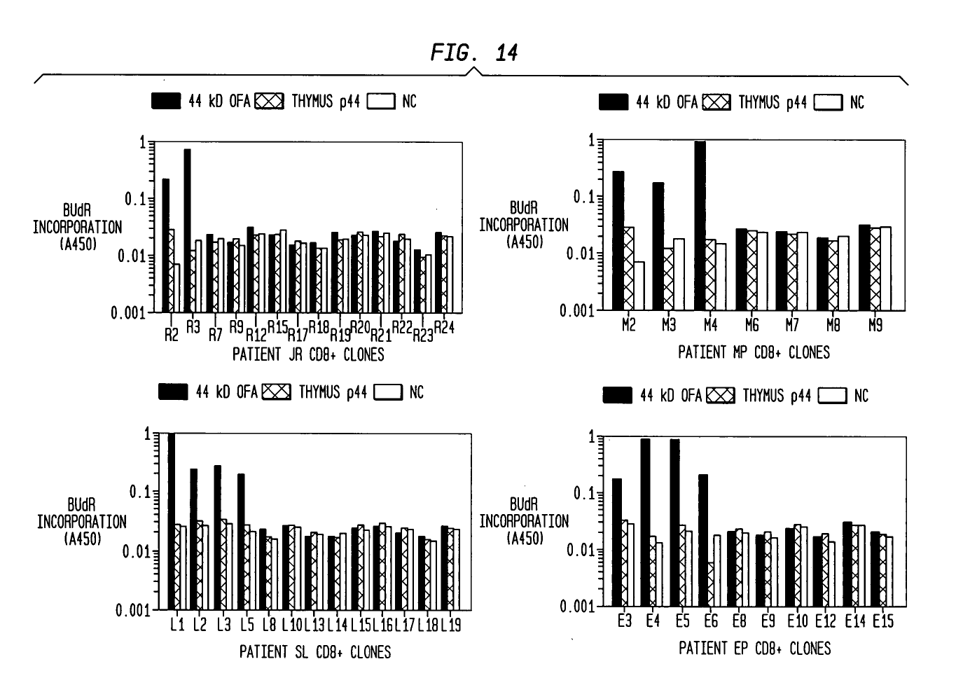

FIG. 13 shows that from 20% to 50% (mean=32.1±7.6%) of the CD4+ tumor-reactive clones from the 4 breast carcinoma patients proliferated specifically to 75 ng/well of purified 44 kD oncofetal antigen protein:nitrocellulose particles. While the OFA was purified from the 5T mouse thymic lymphoma, the proliferation was specific in that there was no incorporation of 5-bromo-deoxyuridine over background levels when the clones were cultured in the presence of 75 ng/well of purified normal murine thymus 44 kD protein (not OFA):nitrocellulose particles or unconjugated nitrocellulose particles. The stimulation index for OFA-reactive CD4 clones to purified 44 kD OFA:nitrocellulose particles compared to the response to bare:nitrocellulose particles was from 34.1 to 65.4 (mean±S.E.M.=49±4.9). FIG. 14 similarly shows that from 14.3% to 42.9% (mean±S.E.M.=32±7.4%) of the CD8+ tumor-reactive clones from the 4 breast carcinoma patients proliferated specifically to 75 ng/well of purified 44 kD oncofetal antigen protein:nitrocellulose particles, but no clones responded above background levels to normal murine thymus p44:nitrocellulose particles. The stimulation index for the OFA-reactive CD8 clones to purified 44 kD OFA:nitrocellulose particles compared to bare nitrocellulose particles was from 9.3 to 68.8 (mean±S.E.M.=28.9±6.2). Culture of the clones with normal thymus p44:nitrocellulose particles induced no more proliferation than with bare nitrocellulose particles (FIGS. 13 and 14). Thus, approximately 30% of the breast carcinoma patients' T cell clones specifically proliferate to OFA presented by irradiated autologous PBML and approximately 70% of the clones respond to some non-OFA epitope on the autologous tumor cells. There also appears to be two populations of CD8 anti-OFA T cells in all patients observed in that some proliferate vigorously to 75 ng/well of OFA while others proliferate approximately only one-seventh as much to the same dose of OFA:nitrocellulose particles. This was also previously seen in irradiated RFM mice which had survived T lymphoma development (27).

Cytokine Profiles of CD4+ OFA-Specific Clones from Breast Carcinoma Patients

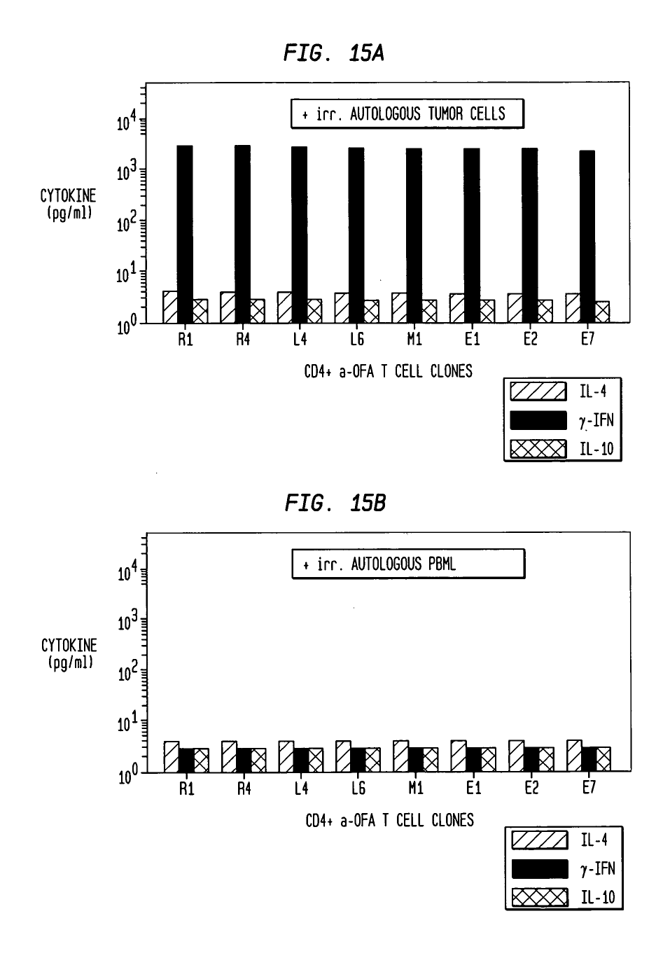

Supernatants taken from 48 hour restimulation cultures of OFA-specific CD4 T cell clones in the presence of recombinant human IL-2 and irradiated T cell-depleted autologous peripheral blood mononuclear cells±irradiated autologous breast carcinoma cells were assayed for γ-IFN, IL-4, and IL-10 by ELISA. None of the CD4 T cell clones from any of the patients secreted amounts of IL-4 or IL-10 above the level of sensitivity while all secreted >2000 pg/ml of γ-IFN (FIG. 15A). That these clones express the cytokine profile of TH1 cells is to be expected since they were initially cultured, cloned, and expanded in the presence of γ-IFN which inhibits the growth of TH2 CD4+ T cells (32). That the interferon was the product of the T cell clones that were being restimulated and is OFA-induced is suggested by the fact that cultures containing the CD4 clones plus irradiated autologous T cell-depleted PBML and IL-2, but lacking the irradiated autologous tumor cells, did not have any γ-IFN detectable above the level of sensitivity of the assay (FIG. 15B).

Cytokine Profiles of CD8+ OFA-Specific Clones from Breast Carcinoma Patients.

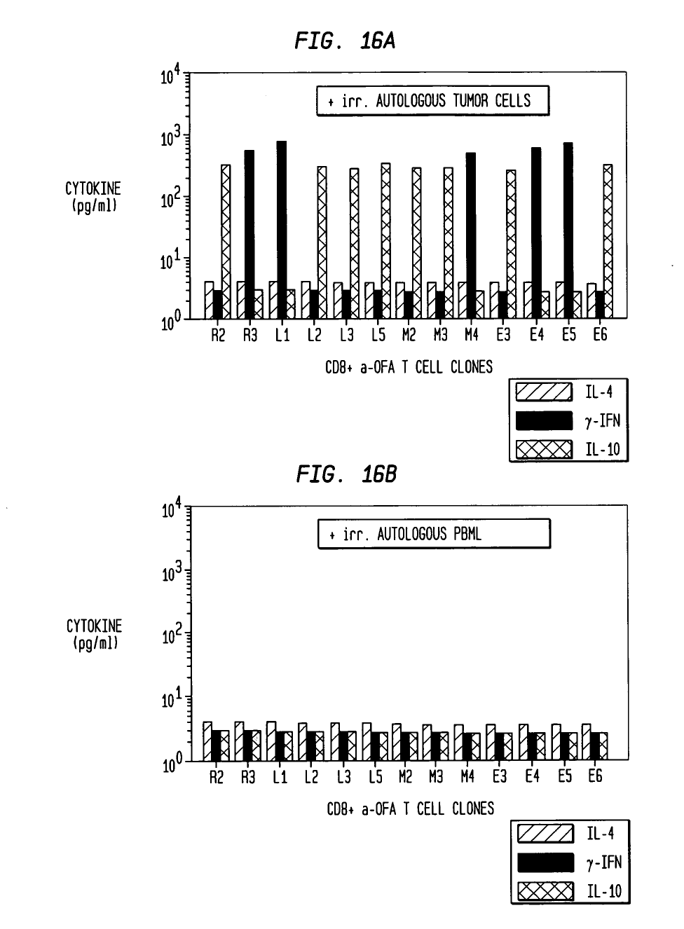

Supernatants taken after 48 hour restimulation cultures of OFA-specific CD8 T cell clones in the presence of recombinant IL-2 and irradiated T cell-depleted autologous PBML±irradiated autologous breast carcinoma cells were assayed as described above for γ-IFN, IL-4, and IL-10. None of the CD8 T cell clones secreted detectable amounts of IL-4. However, two non-overlapping populations of CD8 clones were shown by their IL-10 and γ-IFN secretion profiles (FIG. 16A). Patients JR and EP had 50% of their CD8 OFA-specific clones secreting γ-IFN and 50% secreting IL-10. Patient SL had 3 of 4 CD8 clones secreting IL-10 and patient MP had 2 of 3 CD8 clones secreting IL-10. The clones not secreting IL-10 secreted γ-IFN. Approximately equivalent amounts of cytokine were secreted no matter which cytokine was secreted in that they all secreted between 400 and 1000 pg/ml of either IL-10 or gamma interferon. Thus the CD8 clones established express the cytokine profiles we previously described for murine cytotoxic T cells (γ-IFN) and for non-cytotoxic, inhibitory T cells (IL-10) established from long-term survivors of irradiation-induced lymphomagenesis (27). That the γ-IFN and IL-10 were the products of the T cell clones that were being restimulated and were OFA-induced is suggested by the fact that cultures containing the CD8 clones plus the irradiated autologous T cell-depleted PBML and IL-2, but lacking the irradiated-autologous tumor cells, did not have any γ-IFN or IL-10 detectable over the level of the sensitivity of the assays (FIG. 16B).

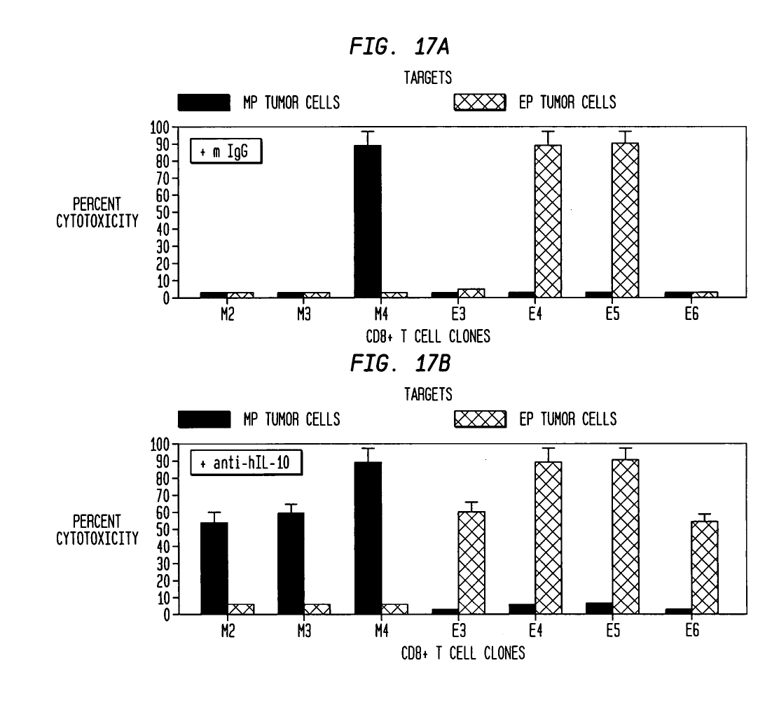

The CD8 OFA-specific clones kill only autologous breast carcinoma cells and that cytotoxicity is inhibited by IL-10 secretion. Because we had previously found that IL-10-secreting clones were T_{C} cells, but could only be shown as such in the presence of neutralizing anti-IL-10 antibody (29) and because autologous tumor cells were limited in number, we combined analysis of the cytotoxic activity of the CD8+ OFA-specific T cell clones from patients MP and EP with analysis of the specificity of the cytotoxicity and the IL-10 inhibitory activity on that cytotoxicity. Thus, the CD8 clones were cultured with autologous or allogeneic breast carcinoma cells at an effector:target ratio of 50:1 for 48 hours±monoclonal anti-human IL-10 or mouse IgG as an isotype antibody control. FIG. 17A shows that only 1 of 3 CD8 MP clones and 2 of 4 CD8 EP clones were cytotoxic to autologous tumor cells in the presence of isotype control antibody. No clones were cytotoxic to non-autologous tumor cells. However, in the presence of anti-IL-10 (FIG. 17B), all CD8 clones were cytotoxic to their autologous tumor cells, but were still not cytotoxic to allogeneic breast carcinoma cells. The clones which required anti-IL-10 to be able to be cytotoxic were less cytotoxic than were those 3 clones that could kill the tumor cells in the absence of anti-IL-10. Those clones which were cytotoxic without any antibody addition being required were also the clones which were secreting γ-IFN (clones M4, E4, and E5). These were also clones that proliferated more vigorously to OFA (S.I. of 62.3, 68.8, and 40.8, respectively). Thus, the CD8+ OFA-specific clones which secrete IL-10 are inhibited from acting as effector cells by the IL-10 they are secreting and, presumably, may act as inhibitors of neighboring effector cells in vivo due to their secretion of IL-10.

Because both patient's CD8 T cell clones kill the autologous tumor cells significantly better (p<0.01) than the other patient's tumor cells and both tumors express OFA (FIG. 11), the cytotoxicity may be MHC-restricted and thus, the clones may be traditional TC cells. This is true for both the IFN-γ-secreting TC clones (FIG. 17A) and for the IL-10-secreting CD8 clones whose cytotoxicity is apparent only when they are assayed in the presence of neutralizing anti-IL-10 antibody (FIG. 17B). While, we do not have the patients' MHC types to directly demonstrate MHC-restriction, the fact that the clones are CD3+, CD8+, TCR+, is suggestive that the requirement for the tumor targets to be autologous to the effector cells in order to get cytotoxicity is due to MHC recognition and restriction of the cytotoxic T cell clones. Human OFA-specific T cell clones recognize immature laminin receptor protein as well as OFA.

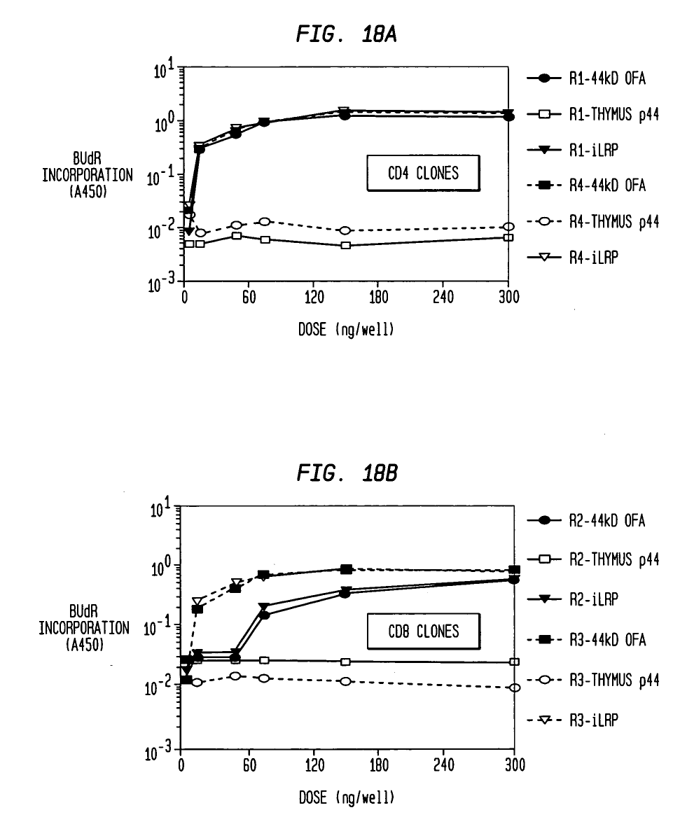

FIG. 18 shows the proliferative response of the CD4 clones (A) of patient JR and the CD8 clones (B) of patient JR to various doses of purified 44 kD OFA, normal thymus 44 kD protein (p44), and recombinant iLRP. While there was no proliferative response to normal thymus 44 kD protein (not OFA) by any of the clones, all of the clones responded by proliferation to both purified 44 kD thymic lymphoma-derived OFA and iLRP. In fact, while 3 of the 4 clones were optimally responding to OFA at a dose of 75 ng/well and the other clone could respond to no less than 75 ng/well, the dose response curve of each to OFA was identical to the dose response curve to iLRP. Thus, the human T cell clones induced during breast carcinoma development that recognized OFA also recognized in a quantitatively identical manner iLRP. The data shown are only those for patient JR's OFA-specific T cell clones, but they are representative of the other three patients' clones as well (data not shown). The data presented herein suggest that similar events may occur during the development of breast carcinomas in humans and show that just as in experimental animals, OFA is an autoimmunogen for T lymphocytes which have potential protective effector activity, but also lead to induction of T lymphocytes which secrete IL-10 that inhibits cytotoxic activity (FIG. 17). While for some tumors, the cytotoxic T cells appear to kill both autologous and allogeneic tumor cells in man (52,53), it is clear from FIGS. 17A and 17B that both the outright cytotoxic CD8 T cells and the TC whose cytotoxicity is blocked by their IL-10 secretion are both able to kill autologous, but not allogeneic breast carcinoma cells even though OFA is expressed by both targets (FIG. 11). Some αβ TCR+ T cells express NK1.1 (54) and appear to be restricted by CD1 instead of MHC (55). Those NK T cells can be important in anti-tumor cytotoxicity (56,57), and in IL-12-induced immunity (58). Therefore, it is important that our OFA-reactive CD8 clones are restricted, at least, to autologous tumor cells (which is probably due to MHC-restriction) and thus are probably classical cytotoxic T cells. Because our clones react to iLRP and iLRP has been shown to induce αβ TCR+ T cells (59), our work shows that typical TCR+ TH1 and TC reactive to OFA and iLRP are induced during breast carcinoma development in humans also.

All patients' tumors in this study expressed OFA which confirmed previous data showing OFA to be present (24,25) and suggesting that it should serve as a TAA in humans as it does in mice (26-29). Also, all patients had peripheral blood T lymphocytes which reacted very vigorously to irradiated autologous tumor cells in the presence of exogenous IL-2, γ-IFN, and IL-6. Unlike the mouse spleen cells taken either from tumor-immune animals or from tumor-surviving animals, the human breast cancer patients' T lymphocytes proliferated enough from the beginning of culture with autologous tumor cells that the culture had to be split within 2-3 days after initiation. After about 12 days, the cultures began to show some cell death which is normal. Subsequent to restimulation with irradiated autologous tumor cells in the presence of the same cytokines as above, limited dilution cloning resulted in from 11-24 tumor-reactive clones. None of the clones subsequently responded by proliferation or cytokine production to culture with irradiated autologous PBML and the cytokines described above unless irradiated autologous tumor cells or purified OFA:nitrocellulose particles were present. They were, thus, operationally defined as tumor-reactive and some of them were reactive to the tumor due to recognition of OFA/iLRP expression by the tumor cells. Of these from 16.7%-43.8% were clones which proliferated specifically to purified 44 kD OFA. When dose responses to OFA were determined it was found that all but the IL-10-secreting clones responded optimally to low doses of OFA. This may suggest that those clones were the progeny of memory T lymphocytes which composed part of the anti-OFA T cell response in vivo that had undergone affinity maturation (60). That the IL-10-secreting clones required higher doses of OFA to respond optimally may suggest that these cells are naive and induced by the tumor to inhibit the anti-tumor immunity. Suppressor T cells often have appeared to be short-lived T cells continually re-seeded from the thymus and so would have shown a reactivity to antigen that appeared due to a low affinity TCR. This same higher affinity interaction of the effector T cells with OFA and an apparent lower affinity interaction by the IL-10-secreting T cells was seen in long-term survivors of X-ray induced lymphomagenesis (27).

Not only was there an apparent affinity maturation in the anti-OFA T cell response yielding T cell clones with higher affinity TCRs during breast carcinoma development, but the CD4:CD8 ratio appeared to be inverted in the tumor-reactive and OFA-reactive clones compared to uncloned peripheral blood T lymphocytes from the same patients. While this may represent an artifact of the cloning procedure, a similar inversion was previously seen in the tumor-reactive clones from lymphomagenesis survivor mice (27). Because OFA is present on early to midgestation fetuses in experimental animals and man, it is of interest that a similar CD8:CD4 inversion occurs during pregnancy in humans concurrent with a reduction in the potency of immunity (61). That this may actually reflect a change in the phenotype of circulating tumor-reactive T cells in vivo during tumor growth is suggested by the observation of a similar inversion during IL-2 treatment of lymphoma- and mastocytoma-bearing mice (62). In that case, an increase in therapeutic, anti-tumor CD8 TC cells occurred at the site of the tumor. Tumor-infiltrating T lymphocytes in human renal cell carcinoma also had a lower CD4:CD8 ratio than is normal (63).

The survival of established tumors in hosts in which concomitant immunity can be demonstrated through adoptive transfer or partial tumor excision and rechallenge with the same tumor at a different site has often been explained by selection for poorly immunogenic tumor cells subsequent to induction of immunity (8) or by tumor-induced immune suppression (12,17). It has been demonstrated that IL-10 is secreted by tumor cells (15). Tumor-infiltrating lymphocytes in human renal cell carcinoma have been shown to have a high frequency of IL-10-secreting cells (64,65) and inhibited in vitro cytotoxicity to autologous tumor cells (66). We have previously shown that the irradiation-induced lymphomagenesis survivor mice CD8 T cell clones which were non-cytotoxic and did not secrete γ-IFN secreted IL-10 which could inhibit cytotoxic T cell activity in vitro and may have explained the enhancement of tumor development in those survivor mice in vivo (27,67). We now find that the same populations of T lymphocytes appear to be clonable from breast carcinoma patients. Also, patients SL and MP who had 75% and 67% of their CD8 clones secreting IL-10 both had to re-enter the hospital for a second mastectomy operation during the course of this study. This indicates that the profile of T cells reactive to OFA is predictive of the therapeutic outcome.

We show (below) that the peptide sequence of 67% of the OFA protein is 100% homologous with the iLRP sequence; that monoclonal a-iLRP antibodies bind OFA and that monoclonal anti-OFA antibodies are inhibited from binding OFA by iLRP. We show herein that the CD4 and CD8 human OFA-reactive T cell clones show the same dose response to iLRP as to purified OFA. Murine a-OFA T cell clones also respond to iLRP. Thus, OFA is iLRP or closely related. Besides the sequence and immunological cross-reactivity, functional and temporal similarities also are found. Oncofetal antigen is detectable on cells exposed to carcinogenic insult before they are histologically visible as transformed cells (28). This protein is also expressed in early to midgestation during fetal development, but not expressed in term fetus, normal neonatal or adult tissues (24). The 32-44 kD immature laminin receptor protein is expressed during the same time frame in fetal development (68), is conserved like OFA between many species (30), and is overexpressed in cancer cells and correlates with their metastatic potential (69).

Immature laminin receptor protein is 32-44 kD, but the precursor LRP appears to dimerize to form one component of the high affinity mature 67 kD LRP (70). This dimer, however, is combined non-covalently with a galactoside-binding protein, galectin-3 to form the mature high affinity LRP (71). During tumor progression, galectin-3 is down-regulated (72), while the 32-44 kD monomeric form is over-expressed as the tumor becomes more aggressive (73-76). Because we have found that iLRP (OFA)-reactive effector and inhibitor T lymphocytes are induced during development of breast carcinomas, it is of interest that the expression of iLRP on breast carcinomas (73,74), carcinoma of the colon, (75) and in uterine adenocarcinoma (76) appears to be associated with poor prognoses for the patients. Thus, the frequency of IL-10-secreting CD8+ a-iLRP T cells is also predictive for success of therapy of such tumors. Indeed, in renal cell carcinomas, TC which have infiltrated the tumor bed are dysfunctional (66,77) and IL-10 is often present in the tumor-bed microenvironment (78) with part of it being produced by the anti-tumor T lymphocytes which infiltrate the tumor site (64,65). Thus, we see in breast carcinomas, a tumor antigen (iLRP), which is important for tumor cell invasiveness, induces potentially protective T lymphocytes, but also induces other T lymphocytes to secrete a cytokine (IL-10) capable of inhibiting the effector T cells.

### REFERENCES I

1. Lotze, M. T. and O. J. Finn. 1990. Recent advances in cellular immunology: Implication for immunity to cancer. Immunol. Today 11:190.
2. Berke, G. 1991. Lymphocyte-triggered internal target cell disintegration. Immunol. Today 12:396.
3. Scott, O. C. A. 1991. Tumor transplantation and tumor immunity. Cancer Res. 51:757.
4. Hewitt, H. B. et al. 1976. A critique of the evidence for tumor of spontaneous origin. Brit. J. Cancer 33:241.
5. Embleton, M. J. 1991. Letter to the Editor. Cancer Res. 51:5433.
6. Van Waes, C., J. L. Urban, J. L. Rothstein, P. L. Ward, and H. Schreiber. 1986. Highly malignant tumor variants retain tumor-specific antigens recognized by T helper cells. J. Exp. Med. 164:1547.
7. Boon, T. 1992. Toward a genetic analysis of tumor antigens. Adv. Cancer Res. 58:177.
8. Restifo, N. P., R. Esquivel, A. C. Asher, H. Stotter, R. J. Barth, J. R. Bennick, J. J. Mule, J. W. Yewdell, and S. A. Rosenberg. 1991. Defective presentation of endogenous antigens by a murine sarcoma:implications for the failure of an anti-tumor response. J. Immunol. 147:1453.
9. Barsoum, A. and J. Coggin, Jr. 1991. Isolation and partial characterization of a soluble oncofetal antigen from murine and human amniotic fluids. Inter. J. Cancer 48:248.
10. Rector, J. T., R. N. Lausch, and J. E. Oakes. 1984. Identification of infected cell-specific monoclonal antibodies and their role in host resistance to ocular herpes simplex virus type 1 infection. J. Gen. Virol. 65:657.
11. Harding, R. A., J. G. McArthur, J. A. Gross, D. H. Raulet, and J. P. Allison. 1992. CD28-mediated signaling co-stimulates murine T cells and prevents induction of energy in T cell clones. Nature 356:607.
12. Vaage, J. 1971. Concomitant immunity and specific depression of immunity by residual or reinjected tumor tissue. Cancer Res. 31:1655.
13. Berg, D. and R. G. Lynch. 1991. Immune dysfunction in mice with plasmacytomas. I. Evidence that transforming growth factor contributes to the altered expression of activation receptors on host B lymphocytes. J. Immunol. 146:2865.
14. Smith, D. R., S. L. Kunkel, M. D. Burdick, C. A. Wilke, M. B. Orringer, R. I. Whyte, and R. M. Strieter. 1994 Production of interleukin-10 by human bronchogenic carcinoma. Am. J. Pathol. 130:136.
15. Yu, A., H. Watts, N. Jaffe, and R. Parkman. 1977. Concomitant presence of tumor-specific cytotoxic and inhibitor lymphocytes in patients with osteogenic sarcoma. N. Engl. J. Med. 297:121.
16. Finke, J. H., A. H. Zea, J. Stanley, C. L. Longo, H. Mizoguchi, R. R. Tubbs, R. H. Wiltrout, J. J. O'Shea, s. Kudoh, E. Klein, R. M. Bukowski and A. C. Ochoa. 1993. Loss of T cell receptor chain and p561ck in T-cells infiltrating human renal cell carcinoma. Cancer Res. 53:5613.
17. Bost, K. L., S. C. Bieglik, and B. M. Jaffe. 1995. Lymphokine mRNA expression by transplantable murine B lymphocyte malignancies:Tumor-derived IL-10 as a possible mechanism for modulating the anti-tumor response. J. Immunol. 154:718.
18. Srivastava, P. S. and L. J. Old. 1988. Individually distinct transplantation antigens of chemically induced mouse tumors. Immunol. Today 9:78.
19. Hellstrom, K. E., I. Hellstrom, and J. P. Brown. 1978. Unique and common tumor-specific transplantation antigens of chemically induced mouse sarcomas. Int. J. Cancer 21:317.
20. De Leo, A. B., H. Shikn, T. Takahashi, M. John, and L. J. Old. 1977. Cell surface antigens on chemically induced sarcomas of the mouse. I. Murine leukemia virus-related antigens and alloantigens of cultured fibroblasts and sarcoma cells: description of a unique antigen on BALB/c MethA sarcoma. J. Exp. Med. 146:720.
21. Leffel, M. S. and J. H. Coggin, Jr. 1977. Common transplantation antigens on 3-MCA-induced sarcomas detected by three methods of assay. Cancer Res. 37:4112.
22. Hellstrom, K. E. and I. Hellstrom. 1967. Cell bound-immunity to autologous and syngeneic mouse tumors induced by methylcholanthrene and plastic discs. Science 156:981.
23. Coggin, J. H., Jr. 1989. Cross-reacting tumor associated transplantation antigen on primary 3-methylcholanthrene-induced BALB/c sarcomas. Mol. Biother. 1:223.
24. Payne, W. J., Jr. and J. H. Coggin, Jr. 1985. Mouse monoclonal antibody to embryonic antigen: Development, cross-reactivity with rodent and human tumors, and preliminary polypeptide characterization. J. Natl. Cancer Inst. 75:527.
25. Gussack, G. S., S. D. Rohrer, R. B. Hester, P. I. Liu, and J. H. Coggin, Jr. 1988. Human squamous cell carcinoma lines express oncofetal antigen 44 kilodalton polypeptide defined by monoclonal antibody to mouse fetus. Cancer 62:57.
26. Barsoum, A. L. and J. H. Coggin, Jr. 1989. Immunogenicity of a soluble, partially purified OFA from murine fibrosarcoma in syngeneic mice. J. Biol. Resp. Modifiers 8:579.
27. Rohrer, J. W., C. Culpepper, A. L. Barsoum, and J. H. Coggin, Jr. 1995. Characterization of RFM mouse T lymphocyte anti-oncofetal antigen immunity in apparent tumor-free, long-term survivors of sublethal X-irradiation by limiting dilution T lymphocyte cloning. J. Immunol. 155:2266.
28. Rohrer, S. D., R. N. Sarli, A. L. Barsoum, R. B. Hester, and J. H. Coggin, Jr. 1992. Expression of 44 kilodalton oncofetal antigen as a premalignancy marker in X-irradiation-induced murine T cell lymphoma. J. Natl. Cancer Inst. 84:602.
29. Rohrer, J. W. and J. H. Coggin, Jr. 1995. CD8 T cell clones inhibit antitumor T cell function by secreting IL-10. J. Immunol. 155:5719.
30. Menard, S., V. Castronovo, E. Tagliabue, and M. E. Sobel. 1997. New insights into the metastasis-associated 67 kD laminin receptor. J. Cell. Biochem. 67:155.
31. Boyum, A. 1968. Isolation of mononuclear cells and granulocytes from human blood. Scand. J. Clin. Lab. Invest. 21, Suppl. 97:77.
32. Gajewski, T. F., and F. W. Fitch. 1988. Antiproliferative effect of IFN-gamma in immune regulation. I. IFN-γ inhibits the proliferation of Th2 but not Th1 murine helper T lymphocyte clones. J. Immunol. 140:4245.
33. Rogers, L. A., A., Zlotnik, F. Lee, and K. Shortman. 1991. The maintenance of lytic specificity during the development of clones of cytotoxic T lymphocytes from single precursor cells. J. Immunol. Methods 143:241.
34. Wysocki, L. J. and V. L. Sato. 1978. Panning for lymphocytes: A method for cell selection. Proc. Natl. Acad. Sci. (USA) 75:2844.
35. Decker, T. and M.-L. Lohmann-Mathes. 1988. A quick and simple method for the quantitation of lactate dehydrogenase release in measurements of cellular cytotoxicity and tumor necrosis factor (TNF) activity. J. Immunol. Methods 15:61.
36. Standring, R. and A. F. Williams. 1978. Glycoproteins and antigens of membranes prepared from rat thymocytes after lysis by shearing or with detergent Tween-40. Biochem. Biophys. Acta 508:85.
37. Laemmli, U. K. 1970. Cleavage of structural proteins during the assembly of the head of bacteriophage T4. Nature 227:680.
38. Towbin, H., T. Staehlin, and J. Gordon. 1979. Electrophoretic transfer of proteins from polyacrylamide gels to nitrocellulose sheets:Procedure and some applications. Proc. Natl. Acad. Sci. (USA) 76:4350.
39. Harlow, E. and L. Lane. 1988. Antibodies: A Laboratory Manual. Cold Spring Harbor Laboratory, Cold Spring Harbor, N.Y., p. 494.
40. Abou-Zeid, C. E. Filley, J. Steele, and G. A. W. Rook. 1987. A simple new method for using antigens separated by polyacrylamide gel electrophoresis to stimulate lymphocytes in vitro after converting bands cut from Western blots into antigen-bearing particles. J. Immunol. Methods 98:5.
41. Wahl, D. V., W. H. Chapman, I. Hellstrom, and K. E. Hellstrom. 1974. Transplantation immunity to individually unique antigens of chemically induced bladder tumors in mice. Int. J. Cancer 14:114.
42. Wepsic, H. T., B. Zbar, H. J. Rapp, and T. Borsos. 1970. Systemic transfer of tumor immunity: Delayed hypersensitivity and suppression of tumor growth. J. Natl. Cancer Inst. 44:955.
43. North, R. H. and D. P. Kirstein. 1977. T cell-mediated concomitant immunity as the expressors of nonspecific immunity to unrelated tumors and bacterial parasites. J. Exp. Med. 145:275.
44. Gershon, R. K., R. L. Carter, and K. Kondo. 1967. On concomitant immunity in tumor-bearing hamsters. Nature 213:674.
45. Chen, L., S. Ashe, W. A. Brady, I. Hellstrom, K. E. Hellstrom, J. A. Ledbetter, P. McGowan, and P. S. Linsley. 1992. Costimulation of anti-tumor immunity by the B7 counterreceptor for the T lymphocyte molecules CD28 and CTLA-4. Cell 71:1093.
46. Traversari, C., van der Bruggen, P., I. F. Luescher, C. Lurquin, P. Chromez, A. Van Pel et al. 1992. A nonapeptide encoded by human MAGE-1 is recognized on HLA-A1 by cytolytic T lymphocytes directed against tumor antigen MZ2-E. J. Exp. Med. 176:1453.
47. Gaugler, B., B. Ban den Eynde, P. van der Bruggen, P. Romero, J. J. Gaforio, E. DePlaen et al. 1993. Human MAGE-3 codes for an antigen recognized on a melanoma by autologous cytolytic T lymphocytes. J. Exp. Med. 179:921.
48. Bernhard, H., J. Karbach, T. Wolfel, P. Busch, S. Storkel, M. Stockle. 1994. Cellular immune response to human renal cell carcinomas:Definition of a common antigen recognized by HLA-A2-restricted cytotoxic T lymphocyte clones. Int. J. Cancer 59:837.
49. Kittlesen, D. J., L. W. Thompson, P. H. Gulden, J. C. a. Skipper, T. A. Colella, J. A. Shabonowitz, D. F. Hunt, V. H. Engelhard, and C. L. Slingluff, Jr. 1998 Human melanoma patients recognize an HLA-A1-restricted CTL epitope from tyrosinase containing two cysteine residues: Implication for tumor vaccine development. J. Immunol. 160:2099.
50. Kawakami, Y., S. Eliyahu, C. H. Delgado, P. F. Robbins, K. Sakaguchi, E. Apella et al. 1994. Identification of a human melanoma antigen recognized by tumor-infiltrating lymphocytes associated with in vivo tumor rejection. Proc. Natl. Acad. Sci. (USA) 91:6458.
51. Rosenberg, S. A., B. S. Packard, P. M. Aebersold, D. Solomon, S. L. Topalian, S. T. Toy, P. Simon, M. T. Lotze, J. C. Yang, C. A. Seipp, C. Simpson, C. Carter, S. Bock, D. Schwartzentruber, J. P. Wei, and D. E. White. 1988. Use of tumor infiltrating lymphocytes and interleukin-2 in the immunotherapy of patients with metastatic melanoma: Preliminary report. N. Eng. J. Med. 319:1676.
52. Kim, T. Y., A. C. Von Eschenbach, M. D. Filaccio, K. Hayakawa, D. R. Parkinson, C. M. Balch, and K. Itoh. 1990. Clonal analysis of lymphocytes from tumor peripheral blood and nontumorous kidney in primary renal cell carcinoma. Cancer Res. 50:5263.
53. Mitropoulos, D., S. Kooi, J. Rodriguez-Villanueva, and C. D. Platsoucas. 1994. Characterization of fresh (uncultured) tumor-in filtrating lymphocytes (TIL) and TIL-derived T cell lines from patients with renal cell carcinoma. Clin. Exp. Immunol. 97:321.
54. MacDonald, H. R. 1995. NK1.1+ T cell receptor-+cells: new clues to their origin, specificity, and function. J. Exp. Med. 182:633.
55. Bendelac, A., O. Lantz, M. E. Quimby, J. W. Yewdell, J. R. Bennink, and R. R. Brutkiewicz. 1995. CD1 recognition by mouse NK1+ T lymphocytes. Science 268:863.
56. Ballas, Z. K. and W. Rasmussen. 1991. Lymphokine activated killer (LAK) cells: VI. NK1.1+, CD3+ LAK effectors are derived from CD4, CD8, NK1.1 precursors. Cell. Immunol. 134:296.
57. Takeda, K., S. Seki, K. Ogasawara, R. Anzai, W. Hashimoto, K. Sugiura, M. Takahashi, M. Satoh, and K. Kumagai. 1996. Liver NK1.1 + CD4+ T cells activated by IL-12 as a major effector in inhibition of experimental tumor metastasis. J. Immunol. 156:3366.
58. Anzai, R., S. Seki, K. Ogasawara, W. Hashimoto, K. Sugiura, M. Sato, K. Kumagai, and K. Takeda. 1996. Interleukin-12 induces cytotoxic NK1+ T cells in the lungs of euthymic and athymic mice. Immunology 88:82.
59. Ferrarini, M., S. Heltai, S. M. Pupa, S. Menard, and M. R. Zocchi. 1996. Killing of laminin receptor-positive human lung cancers by tumor-infiltrating lymphocytes bearing gamma delta+ T cell receptor. J. Natl. Cancer Inst. 88:436.
60. Cerottini, J.-C., and H. R. MacDonald. 1989. The cellular basis of T-cell memory. Ann. Rev. Immunol. 7:77.
61. Barnett, M. A., R. P. Learmouth, E. Pihl, and E. C. Wood. 1983. T helper lymphocyte depression in early human pregnancy. J. Reprod. Immunol. 5:55.
62. Mass, R. A., P. A. M. Roest, M. J. Becker, I. S. Weimar, H. F. J. Dullens, and W. Denotter. 1992 Effector cells of low dose IL-2 immunotherapy in tumor-bearing mice: Tumor cell killing by CD8+ cytotoxic T lymphocytes and macrophages. Immunobiol. 186:214.
63. Elasser-Beile, U., U. Wetterauer, W. Schultze-Seemann, H. Gallati, J. S. Monting, and S. von Kleist. 1996. Analysis of the immune reactivity of infiltrating and peripheral lymphocytes from patients with renal cell carcinoma by measuring cytokine secretion. Cancer Immunol. Immunother. 42:93.
64. Wang, W., C. Redovan, R. Tubbs, T. Olencki, E. Klein, S. Kudoh, J. Finke, and R. M. Bukowski. 1995. Selective cytokine gene expression in renal cell carcinoma tumor cells and tumor-infiltrating lymphocytes. Int. J. Cancer 61:780.
65. Maeurer, M. J., D. M. Martin, C. Castelli, E. Elder, G. Leder, W. J. Storkus, and M. T. Lotze. 1995. Host immune response in renal cell cancer: Interleukin-4 (IL-4) and IL-10 mRNA are frequently detected in freshly collected tumor-infiltrating lymphocytes. Cancer Immunol. Immunother. 41:111.
66. Van den Hove, L. E., S. W. Van Gool, H. Van Poppel, L. Baert, L. Coorevits, B. Van Damme, and J. L. Ceuppens. 1997. Phenotype, cytokine production and cytolytic capacity of fresh (uncultured) tumor-infiltrating T lymphocytes in human renal cell carcinoma. Clin. Exp. Immunol. 109:501.
67. Coggin, J. H., Jr., J. W. Rohrer, and A. L. Barsoum. 1997. A new immunobiological view of radiation-promoted lymphomagenesis. Int. J. Radiat. Biol. 71:81.
68. Van den Brule, F. A., J. Price, M. E. Sobel, R. Lambotte, and V. Castronovo. 1994. Inverse expression of two laminin binding proteins, 67LR and galectin-3, correlates with the invasive phenotype of trophoblastic tissue. Biochem. Biophys. Res. Commun. 201:388.
69. Sobel, M. E. 1993. Differential expression of 67 kD laminin receptor in cancer. Cancer Biol. 4:311.
70. Landowski, T. H., E. A. Dratz, and J. R. Starkey. 1995. Studies of the structure of the metastasis-associated 67 kDa laminin binding protein: Fatty acid acylation and evidence supporting dimerization of the 32 kDa gene product to form the mature protein. Biochem. 34:11276.
71. Castronovo, V. 1993. Laminin receptors and laminin-binding proteins during tumor invasion and metastasis. Invasion Metastasis 13:1.
72. Castronovo, V., F. A. Van Den Brule, P. Jackers, N. Clausse, F. T. Liu, C. Gillet, and M. E. Sobel. 1996. Decreased expression of galectin-3 is associated with progression of human breast cancer. J. Pathol. 179:43.
73. Pellegrini, R., S. Martignone, E. Tagliabue, D. Belotti, R. Bufalino, N. Cascinelli, S. Menard, and M. I. Colnaghi. 1995. Prognostic significance of laminin production in relation with its receptor expression in human breast carcinomas. Breast Cancer Res. Treat. 35:195.
74. Martignone, S., S. Menard, B. Bufalino, N. Cascinelli, R. Pellegrini, E. Tagliabue, S. Andreola, F. Rilke, and M. I. Colnaghi. 1993. Prognostic significance of the 67 kilodalton laminin receptor expression in human breast carcinomas. J. Natl. Cancer Inst. 85:398.
75. Mafune, K., T. S. Ravikumar, J. M. Wong, H. Yow, L. B. Chen, and G. D. Steele, Jr. 1990. Expression of a Mr 32,000 laminin-binding protein messenger RNA in human colon carcinoma correlates with disease progression. Cancer Res. 50:3888.
76. Van den Brule, F. A., C. Cuicu, A. Berchuck, R. C. Bast, M. Deprez, F. T. Liu, D. N. Cooper, C. Pieters, M. E. Sobel, and V. Castronovo. 1996. Expression of the 67-kD laminin receptor, galectin-1, and galectin-3 in advanced human uterine adenocarcinoma. Hum. Pathol. 27:1185.
77. Kudoh, S., C. Redovan, P. Rayman, M. Edinger, R. R. Tubbs, A. Novick, J. H. Finke, and R. M. Bukowski. 1997. Defective granzyme B gene expression and lytic response in T lymphocytes infiltrating human renal cell carcinoma. J. Immunother. 20:479.
78. Angevin, E., F. Kremer, C. Gaudin, T. Hercend, and F. Triebel. 1997. Analysis of T-cell immune response in renal cell carcinoma:polarization to type 1-like differentiation pattern, clonal T-cell expansion and tumor-specific cytotoxicity. Int. J. Cancer 72:431.

### EXAMPLE 29

Immature Laminin Receptor Protein Immunization Induces Anti-Oncofetal Antigen B and T Lymphocyte Immunity

In this example, it is shown that T lymphocyte clones specific for OFA which were established from X-irradiated RFM mice which survived T lymphoma development proliferate equally well to both OFA and iLRP stimulation. Immunization of BALB/c mice with iLRP-conjugated nitrocellulose particles induces IgG antibody which specifically binds iLRP and purified OFA. The amount of anti-iLRP/OFA IgG produced is dependent on the dose of iLRP used to immunize the mice. Both CD4 and CD8 T lymphocyte clones reactive to syngeneic tumor cells were established from iLRP-immune mice. All clones from iLRP-immune mice proliferated specifically to iLRP and OFA. All CD4 clones secreted γ-IFN, but not IL-4 or IL-10. Both γ-IFN-secreting, CD8 TC cells and IL-10-secreting, inhibitory CD8 T cell cells were cloned. The IL-10-secreting CD8 clones were converted to TC cells in the presence of neutralizing anti-IL-10 antibody. Thus, iLRP induces OFA-specific T and B cell responses.

Mice

RFM/UnCR male and female 8-10 week old mice were used as sources of spleen cell antigen-presenting cells for restimulation of RFM OFA-specific T cell clones. BALB/cAnN female 8-10 week old mice were used for immunization with iLRP and subsequent tumor challenge. All mice were purchased from Charles Rivers Breeding Laboratories (Wilmington, Mass.) and were maintained in the vivarium of the Department of Comparative Medicine, University of South Alabama.

Tumor Cells

MCA1315 fibrosarcoma cells (BALB/c) and 5T lymphoma cells (RFM) were grown in RPMI-1640 medium supplemented with essential and nonessential amino acids, sodium pyruvate, sodium bicarbonate, 100 U/ml penicillin G, 100 μg/ml streptomycin sulfate, 10% heat-inactivated fetal calf serum (Sigma Chemical Co., St. Louis, Mo.) (complete RPMI). The cells were maintained at a 37 C humidified 5% C_{O}2/95% air atmosphere. The MCA1315 cells were used for challenge of iLRP-immune mice and for restimulation of T cells derived from those mice for cloning. The 5T lymphoma cells were used for purification of membranes and purified OFA 44 kD protein preparation.

Cell Lines

The gibbon T cell lymphoma MLA-144 [American Type Culture Collection (ATCC), Rockville, Md.] constitutively secretes gibbon IL-2 (32). It was cultured in complete RPMI-1640 and was used as a source of IL-2 for established, expanded clones. The anti-OFA IgM-producing hybridoma 115 (3) is carried as an ascites tumor from which ascites fluid is collected and mAb 115 is purified, as described previously (19). RFM T lymphocyte clones 1, 7, and 9 (which were established from long-term surviving, X-irradiated RFM mice and which appeared to have arisen spontaneously in response to X-ray-induced primary T cell lymphoma development in those mice) were restimulated every two weeks with irradiated RFM 5T lymphoma cells in the presence of irradiated RFM mouse spleen cells in complete RPMI medium supplemented with 10% MLA-144 culture supernatant as a source of IL-2. The phenotype and in vitro activity of these clones has been thoroughly described (13,20).

Anti-T Cell mAbs

Monoclonal anti-CD8 Ab was purified by ammonium sulfate precipitation and protein G affinity chromatography from culture supernatants of hybridoma 53-6.72. Monoclonal anti-CD4 Ab was purified by ammonium sulfate precipitation and protein G affinity chromatography from culture supernatants of hybridoma GK1.5. These hybridomas were obtained from ATCC and are maintained currently in the laboratory. The monoclonal anti-αβ and anti-αβ TCT mAbs from hybridomas H57-597 and GL3, respectively, were obtained from Pharmingen (San Diego, Calif.).

Antigen Preparation

A full-length cDNA of iLRP was cloned into an expression vector under the control of the tac promoter and expressed in E. coli. Inclusion bodies were isolated and solubilized in 6M guanidine hydrochloride in 20 mM Tris, pH 8.0, 0.1 M NaCl, 2 mM EDTA, 0.2% sodium azide. The solubilized protein was renatured and dialyzed against 20 mM Tris, pH 8.0, 0.1 M NaCl, and 0.04% sodium azide. The purity of riLRP was checked by SDS-PAGE. Only one band was seen after staining with Coomassie R250. For the preparation of Ag-bearing NC particles, the method of Abou-Zeid et al. (33) was used as described previously (20). Briefly, solubilized riLRP was subjected to 12% SDS-PAGE according to the method of Laemmli (34) and separated proteins were transferred to NC (35), made visible by staining with Ponceau S (36) and the NC bands carrying the riLRP were cut. The NC bands were dissolved and sterilized in DMSO during 1 h incubation at room temperature. Precipitation was subsequently obtained by the dropwise addition of 0.05 M carbonate/bicarbonate (pH 9.6) while shaking continuously. Each suspension was washed three times with sterile PBS. Purified 44 kD OFA from RFM 5T lymphoma cells was purified as previously described (19).

A 44 kD protein was purified from normal RFM mouse thymus as a non-OFA negative control for proliferation assays. After CO2 asphyxiation of RFM mice, their thymuses were removed, washed in cold Tris-buffered saline, pH 7.4, and were minced with scissors. Thymus tissues were homogenized in 0.5% NP40 in TBS containing the following protease inhibitors: aprotinin (100 KIU/ml), phenylmethylsulfonylfluoride (PMSF, 1 μM), leupeptin (15 μM), N-α-p-tosyl-L-lysine chloromethyl ketone (TLCK, 50 μM), and soybean trypsin inhibitor (5 μg) in an Elvenheim glass homogenizer, and the homogenates were kept for an hour on ice. The mixtures were then centrifuged for 15 minutes at 2,000×g to remove the nuclei and for 1 hr. at 100,000×g to remove insoluble material. Protein concentration of the NP40 extract was determined using the BCA method (37) and adjusted to 1 mg/ml. The NP40 extract was mixed with an equal volume of 2× Laemmli's sample buffer (34), boiled for 5 min., then SDS-PAGE, Western blotting on nitrocellulose, and staining of the blot with Ponceau S, as described above in this section was done.

After staining, the nitocellulose with Ponceau S, a band corresponding in molecular weight to 44 kD riLRP/OFA was cut with a clean scalpel, solubilized in DMSO and processed for the production of fine antigen-bearing particles as described above using the procedure of Abou-Zeid et al. (33).

For testing the specificity of the established clone proliferation to riLRP, the same procedure for making antigen-bearing NC particles was used except that purified recombinant Brucella abortus protein BCS30 (38), r firefly luciferase, r BAG-1 (39), r OB (leptin), r human IL-9 receptor and riLRP that had been affinity purified with anti-iLRP monoclonal IgG antibodies from hybridomas 43515, 43519, and 43532.

Biotinylation of IgG Fraction

Biotinylation of mouse anti-riLRP IgG was performed as described previously (40). In brief, 1 mg/ml of IgG was dialyzed against 0.1 M NaHCO3 overnight and was incubated on a shaking platform with 60 μg of N-hydroxysuccinimido-biotin (Pierce Chemical Co., Rockford, Ill.; which was dissolved in dimethyl sulfoxide) for 4 hr at 25° C. Thereafter, the mixture was dialyzed extensively with PBS for 60 hr, and the buffer was changed six times. The protein concentration of the biotinylated IgG was measured and stored at 4° C. until used.

SDS-PAGE and Western Blotting of thymocyte extract, purified OFA and riLRP. The NP40 thymocyte extract was mixed with an equal volume of 2× Laemmli's sample buffer (34), boiled for 5 min and applied (50 μg/lane) to 12% homogeneous gel (Bio-Rad Laboratories, Hercules, Calif.). Purified native OFA and riLRP (5 μg/lane) were also applied on separate lanes on the same gel. After electrophoresis, Western blotting on nitrocellulose membranes (Schleicher and Schuell, Keene, N.H.) was performed (35). After blocking of the unoccupied sites with blocking buffer (100 mM NaCl, 10 mM Tris, pH 7.4) containing 5% nonfat dry milk, the membrane was probed with biotinylated murine anti-riLRP IgG diluted (1 μg/ml) in 20 mM Tris-HCl, pH 7.8, 150 mM NaCl, and 0.05% Tween-20 containing 1% BSA for 1 hour at room temperature. After extensive washing, immunoreactivity was detected using the ABComplex (Vector Labs, Burlingame, Calif.), which was developed with both 3,3′-diaminobenzidine and hydrogen peroxide (Bio-Rad Laboratories, Richmond, Calif.) according to the instructions of the manufacturer.

Immunization of BALB/c Mice

Mice were divided into 3 groups and each group (n=5) was immunized i.p. twice, separated by 14 days with either bare NC particles, NC particles to which was bound 1 μg or 10 μg of riLRP suspended in 1 ml PBS. The amount of nitrocellulose/mouse was kept constant (50 mg). Two weeks after the second injection, the mice were bled from the retro-orbital plexus using capillary tubes. One mouse/group had its spleen harvested and used for establishment of tumor-reactive T lymphocyte clones.

T Cell Clone Production

Spleens were excised from BALB/c mice two weeks after their second i.p. injection with NC, 1 μg of iLRP:NC, or 10 μg of iLRP:NC particles. Splenic T lymphocytes were stimulated in vitro with irradiated BALB/c MCA1315 fibrosarcoma cells and the reactive cells cloned by limiting dilution using a modification of the method previously published (13). Essentially the same technique was used except that the culture medium was complete RPMI-1640 medium instead of Iscove's Modified Dulbecco's Medium. The cells were cultured in this medium supplemented with 100 U/ml of recombinant mouse IL-2, 10 U/ml of recombinant mouse IFN-γ, and 10 U/ml of recombinant mouse IL-6 during initial stimulation, cloning, and expansion of the harvested clones that were established. The IL-2 was used as a growth factor for the T cells, IFN-γ was used to inhibit outgrowth of TH2 helper T cells for Ab production (41), and IL-6 was used to promote outgrowth and function of TC cells (42). After growth of the expanded clones had stabilized, sterile filtered MLA-144 culture supernatant was used as the source of IL-2 (at 10% v/v) and no IFN-γ or IL-6 was added. These clones had to be re-stimulated with irradiated OFA+ MCA1315 cells every 2 wk in the presence or irradiated syngeneic spleen cells and complete RPMI-1640 supplemented with 10% v/v MLA-144 supernatant to maintain viability and proliferation.

Determination of T Cell Clone Surface Ag Phenotype by mAb and Complement Depletion

One week after Ag restimulation, part of each T cell clone culture was harvested, washed 3 times by centrifugation in complete RPMI-1640, and a viability count done. The cells were diluted to 1×106 viable cells/ml and their surface Ag phenotype was determined by cytotoxicity with mAbs+facilitating anti-Ig antisera+complement, as previously described (20). The counted cells were pelleted and resuspended in 1 ml anti-CD4, anti-CD8, anti-αβ TCR, or anti-αβ TCR Ab diluted optimally in complete RPMI-1640. For all Abs used, the optimal dilution was 1:15. Control antibody for the anti-CD4 (a rat IgG2b) and anti-CD8 (a rat IgG2a) was normal rat IgG. Similarly, the control for the hamster IgG monoclonals against mouse TCRs was normal hamster IgG. The normal IgGs were obtained from Organon Technika (West Chester, Pa.). After Ab and complement treatment, cells were pelleted by centrifugation, washed 3 times in complete RPMI-1640, and resuspended in 1 ml of complete RPMI-1640. A viability count was done by Trypan blue dye exclusion. The percentage of cells specifically killed or lysed by the experimental Ab and complement treatment was calculated by knowing the number of total and viable cells in each tube at the beginning and comparing the non-specific killing effect of the control Abs+facilitating antiserum+complement with the killing by the experimental Abs+facilitating antiserum+complement treatment.

Determination of T Cell Clone Specificity by Proliferation in Response to Ag

At the time of the 2 wk re-stimulation of the clones to maintain their proliferation, the cloned cells were harvested, washed in complete RPMI-1640, and a viability count done. A portion of the cells was saved to be used in the proliferation assay. The proliferation assay was done with 10,000 viable cloned cells/well+irradiated syngeneic spleen cells+various doses of purified 44 kD OFA protein from RFM 5T lymphoma cells, a purified 44 kD protein from normal RFM thymus (not OFA), recombinant murine immature laminin receptor protein (iLRP) or various control proteins bound to nitrocellulose particles or an equivalent amount of unconjugated nitrocellulose particles in 96 well plates. All wells contained complete RPMI-1640 medium supplemented with 100 U/ml of recombinant mouse IL-2. After 24 hours of culture at 37 C in 95% air/5% CO2 humidified atmosphere, 10 μl of 5-bromodeoxyuridine (BUdR) is added to each well to a final concentration of 10 μM BUdR/well. The cells are then incubated for another 24 hours as before. Proliferation is assayed using the Biotrak bromodeoxyuridine incorporation assay (Amersham, Arlington Heights, Ill.). At the end of the second incubation, the plates are centrifuged at 300×g for 10 minutes and the labeling medium removed. The cells are dried for 1 hour at 60 C, fixed with an ethanol fixative for 30 minutes at RT and then the fixative is removed and blocking buffer (1% protein in 50 mM Tris-HCl; 150 mM NaCl, pH 7.4) is added. The cells are incubated for 30 minutes at RT, the blocking buffer removed and 100 μl of 1:100 diluted peroxidase-labeled anti-BUdR added to each well and the plates incubated for 90 minutes at RT. The antibody solution is then removed and the wells washed 3 times with 300 μl/well of wash buffer. 200 μl of 3,3′,5,5′-tetramethylbenzidine (TMB) in 15% (v/v) DMSO is added to each well and the plate covered and incubated for 5-30 minutes at RT. When the required color density is reached, the reaction is stopped by adding 25 μl of 1M sulphuric acid to each well and the plate read on a microELISA reader at 450 nm.

ELISA of IFN-γ, IL-4, and IL-10 Production by T Cell Clones

Cytokine assay kits for murine IFN-γ, IL-4, and IL-10 from R&D Systems (Minneapolis, Minn.) were used. They utilize horseradish peroxidase-labeled anti-cytokine antibody to detect cytokine captured on the anti-cytokine Ab-coated plates. TMB is the substrate that is added and the color reaction is stopped with 2 N sulphuric acid and the color read at 450 nm. The IFN-γ standard curve was linear between 5 pg/ml and 500 pg/ml and the minimum amount detectable was 2 pg/ml. The IL-4 standard curve was linear between 8 pg/ml and 500 pg/ml and the minimum amount detectable was 2 pg/ml. The IL-10 standard curve was linear between 20 pg/ml and 1000 pg/ml and the minimum amount detectable was 5 pg/ml.

Determination of T Cell Clones' Cytotoxic T Cell Activity Against Syngeneic MCA1315 Fibrosarcoma Cells

Cytotoxicity assays were performed using the CytoTox96 nonradioactive cytotoxicity assay kit produced by Promega (Fisher Scientific, Norcross, Ga.). The assay quantitatively measures lactate dehydrogehase (LDH), a stable cytosolic enzyme that is released upon cell lysis. Released LDH in culture supernatants is measured with a 30 min. coupled enzymatic assay resulting in the conversion of a tetrazolium salt to a red formazan product (43). The amount of color formed is proportional to the number of lysed cells. Color was quantitated using a Titertek Multiskan MC ELISA reader (Fisher Scientific, Norcross, Ga.) which measured absorbance at 492 nm. The setup of the assay was the same as previously described for testing RFM mouse T cell clone cytotoxicity against RFM thymic lymphoma cells (13) except that the medium used was RPMI-1640 and both the clones and the target cells are from BALB/c mice. All cytotoxicity assays were done with 10,000 irradiated MCA1315 cells/well and an effector to target ratio of 50:1 in 96 well plates. Control wells were set up to account for spontaneous LDH release from effectors, spontaneous LDH release from targets, and maximal LDH release from targets as well as the experimental wells. The percent specific cytotoxicity was calculated using the formula listed below:

$$\% Cytotoxicity = \frac{( Experimental - Effector Spontaneous ) - Target Spontaneous}{Target Maximum - Target Spontaneous}$$

There is much less of a spontaneous release of LDH in this assay than of 51Cr in a traditional 51Cr release cytotoxicity assay and, therefore, higher specific cytotoxicity percentages are achieved.

Determination of the Ability of Anti-IL-10 to Convert Noncytotoxic CD8, iLRP-Immune T Cell Clones to Cytotoxic Clones

To determine whether the IL-10-secreting, CD8 T cell clones from iLRP-immune mice were inhibited from cytotoxic activity against OFA+ syngeneic tumor cells by the IL-10 they were secreting, the cells were harvested one day before the normal 2 week restimulation culture and set up in complete RPMI-1640 medium containing 100 U/ml recombinant murine IL-2 and 10 μg/ml rat monoclonal anti-mouse IL-10 IgM (clone AB-71-005; BioSource International, Camarillo, Calif.) or rat monoclonal anti-B220 IgM as a control antibody. The cells were cultured for 24 hours as described previously (44) and then harvested, washed 3 times with complete RPMI-1640 medium and viability counts done. The cells were then diluted appropriately and added to a 4 hour cytotoxicity assay against syngeneic MCA1315 fibrosarcoma cells as described above except that anti-IL-10 or control IgM was added to a final concentration of 10 μg/ml in the cytotoxicity assays.

Results

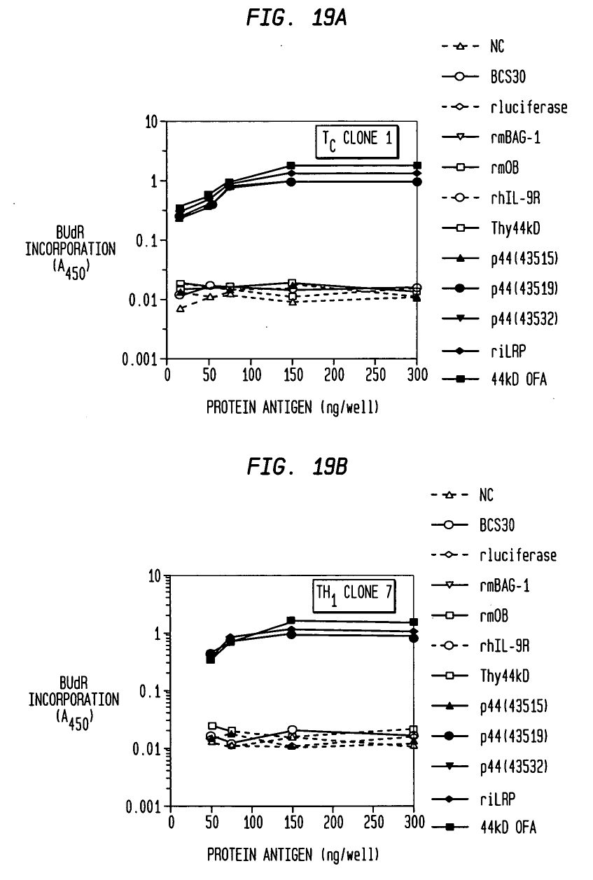

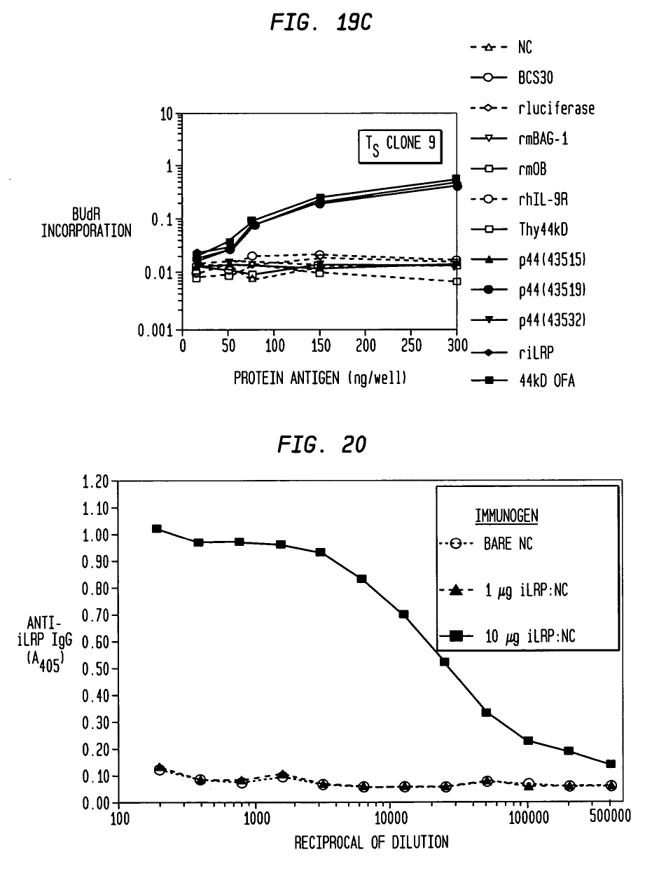

RFM mouse OFA-specific T cell clones proliferate specifically to both purified OFA and recombinant immature laminin receptor protein (riLRP). Stable CD4 and CD8 T cell clones established from RFM mouse long-term survivors of X-irradiation-induced lymphomagenesis which were specific for OFA, as previously observed (13), were cultured in the presence of various doses of purified OFA, recombinant iLRP, or various other control proteins. FIG. 19 shows that OFA-specific cytotoxic CD8+ T (TC) cell clone 1(A), the CD4+ TH1 T cell clone 7(B), and the CD8+ IL-10-secreting, inhibitory TS cell clone 9(C) all proliferated robustly only in response to purified OFA, recombinant iLRP, or various monoclonal anti-iLRP affinity-purified 44 kD iLRP preparations bound to nitrocellulose particles. None of the clones responded more to any of the OFA-negative control proteins at any dose tested than to bare nitrocellulose particles or to a 44 kD protein purified from normal RFM mouse thymus (which is not OFA) (p>0.85). Also, the dose response proliferation to any of the iLRP:nitrocellulose particles was the same as that to OFA:nitrocellulose particles. Therefore, while the TC clone 1 and TH1 clone 7 both responded to OFA and iLRP at a dose as low as 15 ng/well and had an optimal response to both protein preparations at 150 ng/well, the IL-10-secreting inhibitory T cell clone 9 did not respond significantly to a dose of OFA: or iLRP:nitrocellulose particles less than 75 ng/well and had not reached an optimal response at 300 ng/well. Thus, though the TCR affinity may be different from clone to clone, each OFA-reactive clone responds the same to OFA as it does to iLRP. While these dose response differences between the clones to OFA were seen earlier (13), this figure shows that the same dose response difference occurs when iLRP is the stimulating antigen.

Immunization of BALB/c Mice with Recombinant iLRP:Nitrocellulose Particles Induces IgG Anti-iLRP Antibody and that Antibody Specifically Binds to both riLRP and OFA.

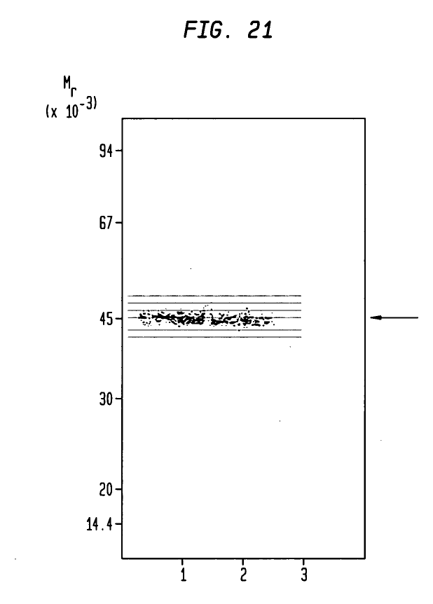

When sera from BALB/c mice which had been injected twice at two week intervals with bare nitrocellulose particles or various doses of iLRP bound to nitrocellulose particles were collected two weeks after the last immunization and assayed for anti-riLRP IgG by ELISA, it is found that no detectable IgG anti-riLRP antibody is induced by bare nitrocellulose or 1 μg of iLRP, but significant anti-riLRP IgG is induced by immunization with 10 μg (FIG. 20). The half maximal titers for mice immunized with NC, 1 μg iLRP:NC, and 10 μg iLRP:NC are <200, <200, and 25,600. Thus, immunization with iLRP induces IgG anti-OFA antibody, but that response is dependent on the dose of iLRP used for immunization. FIG. 21 shows that with Western blot analysis, the anti-riLRP IgG antibody binds equivalently to both riLRP and to purified murine OFA, but does not bind to a detectable amount to any protein in a normal thymus extract. Thus, immunization with riLRP induces IgG antibody which recognizes not only riLRP, but also purified OFA.

Immunization of BALB/c Mice with iLRP:NC Particles Induces MCA1315-Reactive, CD4 and CD8 T Lymphocyte Clones

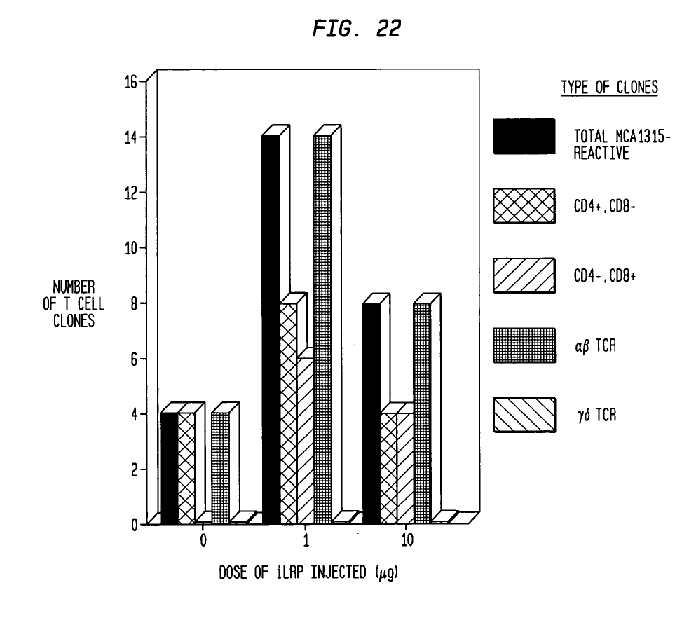

As immunization with different doses of iLRP:NC particles induced different antibody responses and different resistances to syngeneic tumor challenge, they also induced different numbers and types of T lymphocytes reactive to OFA+ MCA1315 fibrosarcoma cells (FIG. 22). Four tumor-reactive T cell clones were able to be established from BALB/c mice injected with bare nitrocellulose particles and all were CD4+, CD8 T cells. Mice injected with 1 μg of iLRP:NC particles yielded 8 CD4+, CD8 and 6 CD4, CD8+ MCA1315 tumor-reactive T cell clones. Immunization with 10 μg iLRP:NC particles yielded 4 CD4+, CD8 and 4 CD4, CD8+ tumor-reactive T cell clones. All clones that were established expressed TCRs (FIG. 22). Immunization of BALB/c mice with iLRP:NC particles induces OFA-reactive T lymphocyte clones.

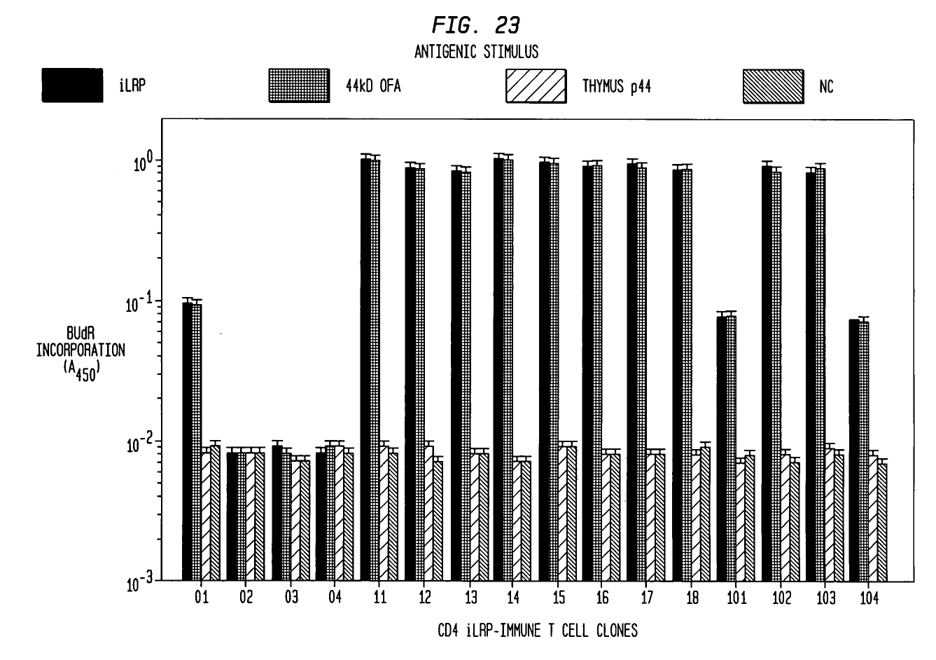

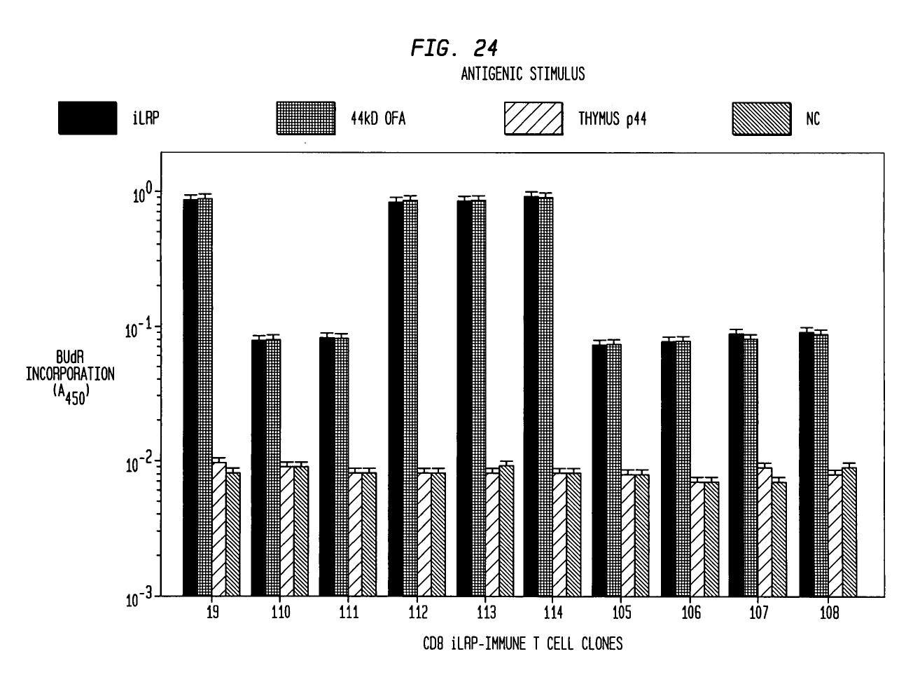

FIGS. 23 and 24 show that while all tumor-reactive clones from mice injected with iLRP:NC particles proliferated specifically in response to 75 ng/well of iLRP:NC or 44 kD OFA:NC particles, only one of the 4 tumor-reactive clones established from the mouse that was injected with bare nitrocellulose particles (clone 01) proliferated to 75 ng/well of iLRP:NC and 44 kD OFA:NC (FIG. 23). That clone incorporated about 10 times less BUdR in response to iLRP:NC and to OFA:NC than did most of the CD4 clones from mice injected with iLRP:NC particles (FIG. 23). Similarly, two of the CD4 clones from 10 μg iLRP:NC particle-injected mice (clones 101 and 104) proliferated about 10 times less to 75 ng/well of iLRP:NC particles in the presence of irradiated, syngeneic spleen cells and IL-2 than the other two clones from that mouse or any of the CD4 clones from the mouse injected with 1 μg iLRP:NC particles when similarly challenged (FIG. 23). There were no CD8 clones from the bare NC particle-injected mouse (FIG. 24). The mouse which was injected with 1 μg of iLRP:NC particles had 2 clones (clones 110 and 111) that responded about 10 times less than the other CD8 or CD4 clones from that mouse to 75 ng/well of iLRP:NC particles in the presence of irradiated syngeneic spleen cells and IL-2 (FIGS. 23 and 24). All of the CD8 clones from the mouse injected with 10 μg of iLRP:NC particles responded about 10 times less well to 75 ng/well of iLRP:NC particles than did the clones which responded well to that dose of 75 ng/well of iLRP:NC particles established from other mice (FIG. 24). While there was a difference in the amount of proliferation various CD4 and CD8 clones had in response to iLRP protein:NC particles, all of the clones proliferated to 75 ng/well of purified OFA to the same extent they proliferated to 75 ng/well of iLRP:NC particles.

That the proliferation to these proteins is specific is shown by the fact that only baseline BUdR incorporation was seen in response to 75 ng/well of normal thymus p44 (not OFA):NC particles and to bare nitrocellulose particles (FIGS. 23 and 24). This indicates that these iLRP-induced T cell clones recognize OFA and iLRP interchangeably, like the iLRP-induced IgG antibodies (FIG. 21).

CD4 T Cell Clones which Respond to iLRP:NC Particles all Secrete Interferon-γ

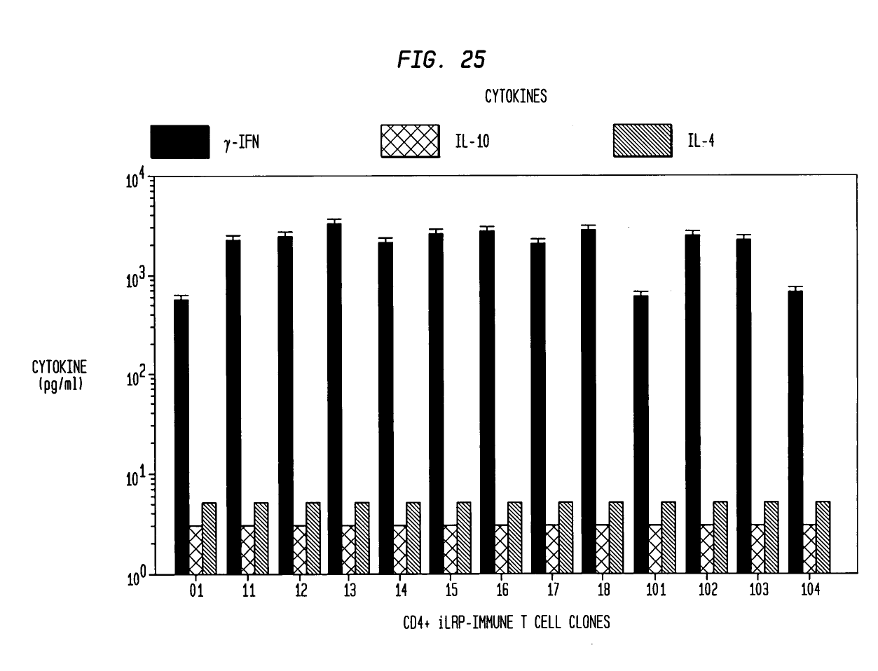

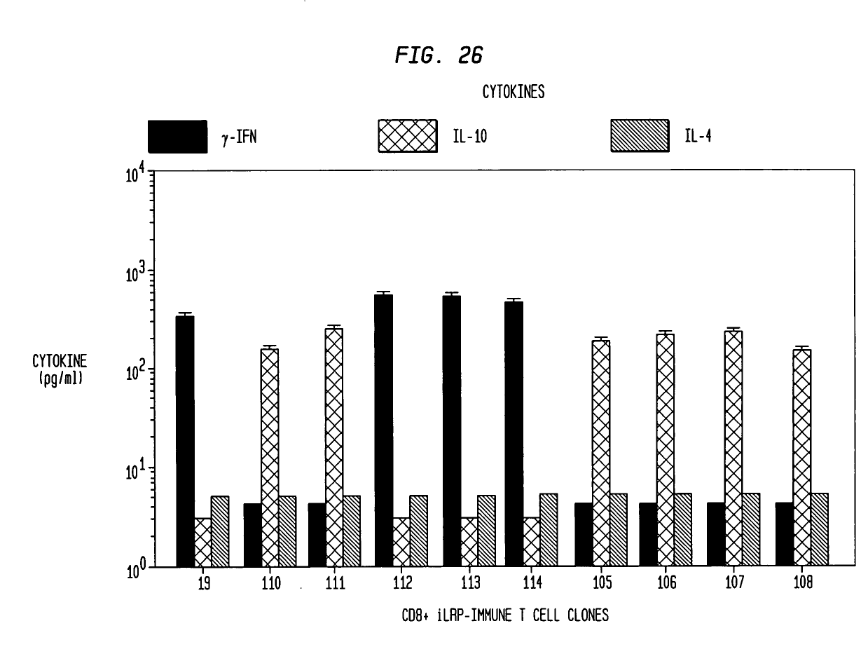

FIG. 25 shows that culture supernatants taken 1 week after restimulation of iLRP:NC-reactive clones with MCA1315 cells all contain >500 pg/ml of IFN-γ. However, just as there were differences in the amount of BUdR incorporated in response to 75 ng/well of iLRP:NC particles among the clones, similar differences in the amount of IFN-γ secreted among the clones subsequent to restimulation by tumor cells is also seen. While all of the CD4 clones from 1 μg iLRP:NC injected mice proliferate well to 75 ng of iLRP:NC (FIG. 24A), they also all secrete >2000 pg/ml of IFN-γ (clones 11-18) (FIG. 25). However, the CD4 clones from bare NC- and 10 μg iLRP:NC particle-injected mice which proliferated less well to iLRP:NC particles (FIG. 23) also produced only 500 to 700 pg/ml of IFN-γ (clones 01, 101, and 104) (FIG. 25). CD8 T cell clones that proliferate to iLRP:NC particles produce either interferon-γ or IL-10. While all the CD4 clones appear to be TH1 cells in that they secrete IFN-γ, but not IL-4 or IL-10 (FIG. 25), the cytokine profiles for iLRP:NC particle-reactive CD8 clones from iLRP:NC-injected mice show two distinct populations. Clones 19 and 112-114 from the mouse which was injected with 1 μg iLRP:NC particles secrete 300-550 pg/ml of interferon-γ while clones 110 and 111 from that same mouse do not secrete IFN-γ, but secrete 150-250 pg/ml of IL-10 (FIG. 26). All of the iLRP-reactive CD8 clones from the mouse injected with 10 μg of iLRP:NC particles (clones 105-108) secrete no detectable IFN-γ, but do secrete 150-250 pg/ml of IL-10 (FIG. 26). The IL-10-secreting CD8 clones are the CD8 clones that proliferated about tenfold less to 75 ng/well of iLRP:NC particles or purified OFA:NC particles than did the other CD8 clones (FIG. 24). There lower responsiveness by proliferation is mirrored by their lower responsiveness measured by cytokine (IL-10, in this case) secretion also.

Control experiments to be sure that the cytokines measured in these cultures were produced by the clones instead of the irradiated tumor cells or irradiated antigen-presenting cells were performed by re-stimulating the CD8 clones in the presence of irradiated tumor cells and irradiated syngeneic, T cell depleted spleen cells for 5 days. At the end of that time, the CD3 (non-T cell) population was separated from the CD3+ (T cell) population, washed and cultured for another 48 hours.

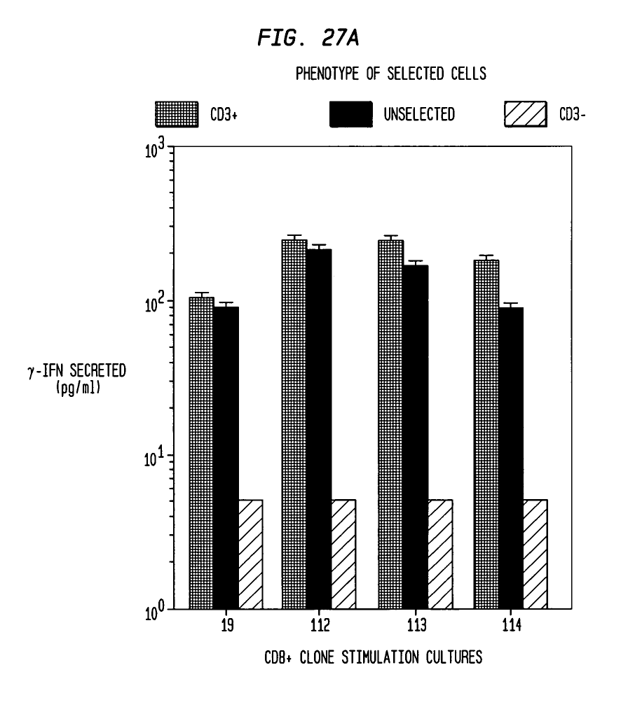

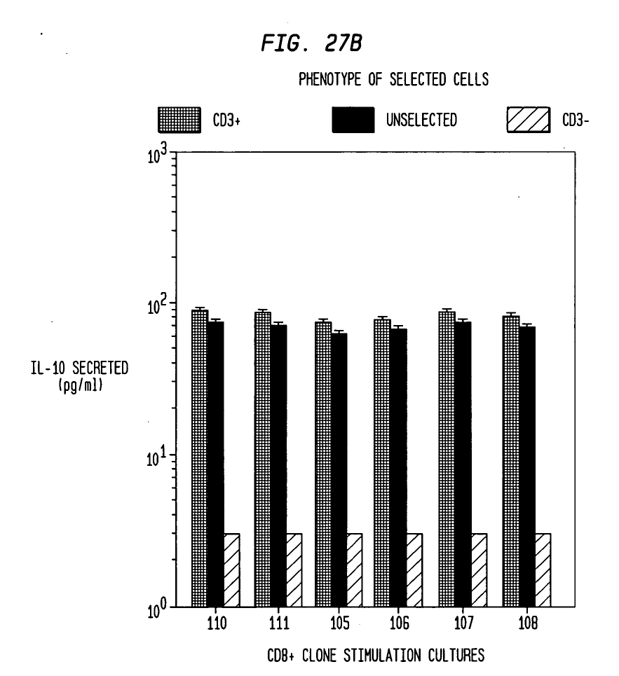

FIGS. 27A and 27B show that only the positively-selected T cells and not the non-T cell components of the stimulation cultures produced detectable amounts of IFN-γ or IL-10 in these experiments. We have previously shown that RFM mouse thymic lymphoma cells are not the source of IL-10 in similar restimulation cultures with OFA-specific, CD8 RFM T cell clones (44), but these data were collected for we did not know if MCA1315 fibrosarcoma cells might be the source in these experiments. Thus the cloned T cells are producing the cytokines. The CD8 T cell clones which secrete IFN-γ, but not the IL-10-secreting CD8 clones, are cytotoxic for MCA1315 fibrosarcoma cells.

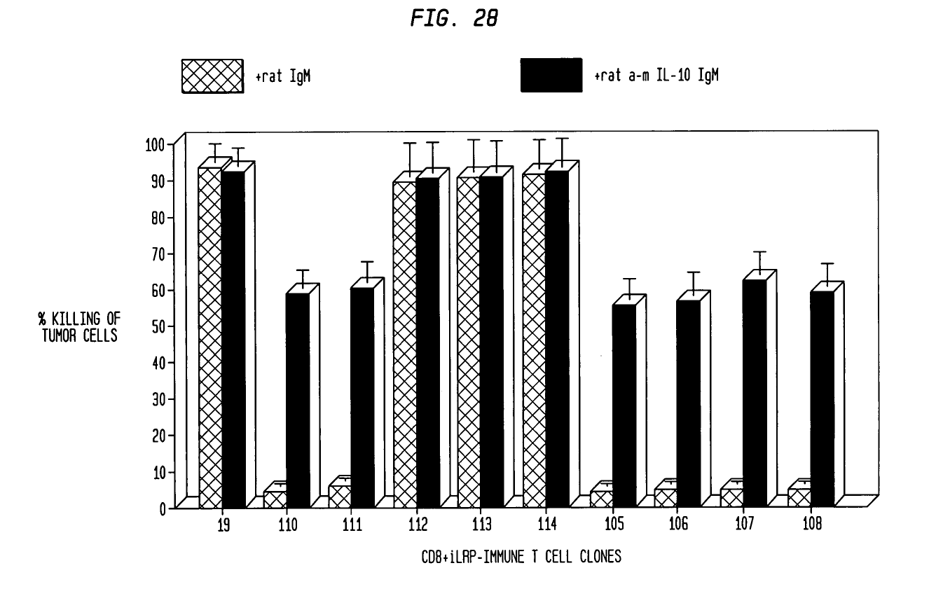

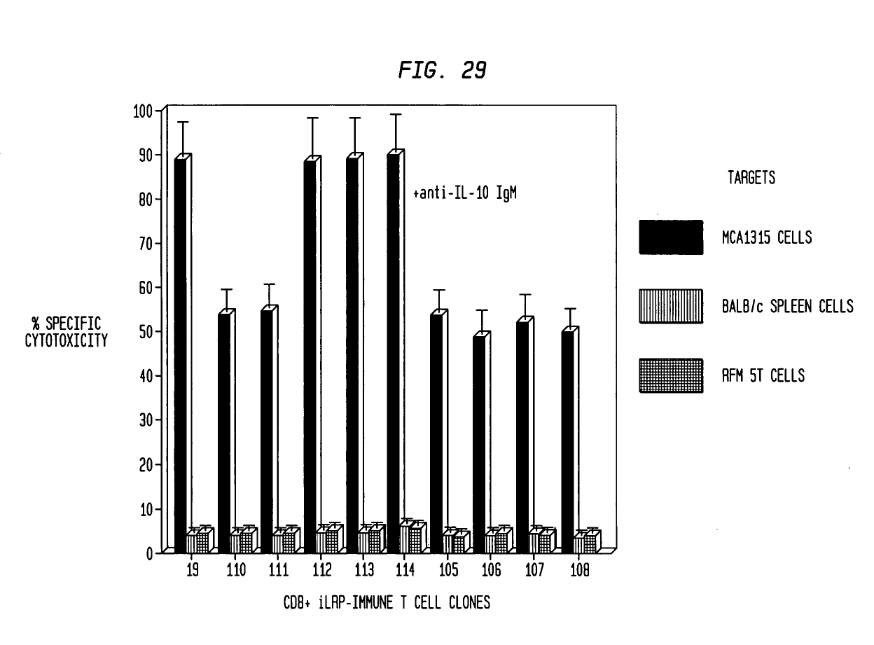

FIG. 28 shows that the BALB/c CD8 T cell clones that secrete IFN-γ (clones 19, 112-114) when incubated with irradiated MCA1315 cells killed those tumor cells. That this was specific cytotoxicity is shown by the fact that those clones did not kill syngeneic normal (OFA) spleen cells nor did they kill irradiated allogeneic OFA+ H-2f T lymphoma cells (FIG. 29). However, the CD8 clones which were found to secrete IL-10 upon antigen stimulation did not kill either the syngeneic or allogeneic tumor cells (both OFA+) or the normal syngeneic spleen cells (OFA) (FIGS. 28 and 29). However, if neutralizing anti-IL-10 antibody is incubated with the cells for 24 hours before and during the cytotoxicity assay, the previously non-cytotoxic CD8 T cells (clones 110, 111, and 105-108) are able to specifically kill OFA+ syngeneic MCA1315 fibrosarcoma cells (FIG. 28). The anti-IL-10, however had no effect on the cytotoxicity observed with the IFN-γ-secreting, cytotoxic CD8 clones.

In conclusion, OFA-specific TH1, TC, and IL-10-secreting TS cell clones established from long-term RFM mouse survivors of X-irradiation-induced lymphomagenesis showed identical proliferation dose responses to purified OFA and recombinant iLRP purified by several monoclonal antibodies while they did not respond to a number of control recombinant proteins or purified 44 kD protein from normal (OFA) RFM thymus. Similarly we showed that immunization of BALB/c mice with recombinant iLRP bound to nitrocellulose particles induced TH1, TC, and counteracting IL-10-secreting TS cell clones depending on the dose of iLRP used for immunization. Indeed, in the mice immunized with the least iLRP which produced no detectable anti-iLRP IgG, but had the most potentially protective iLRP-specific TC clones established, the fewest IL-10-secreting CD8 T cells were cloned. This agrees with our previous suggestion that induction of such suppressors along with or preferentially to effectors could lead to reduced protection subsequent to vaccination (30,31,44). It is important to recognize that a failure to induce solid tumor protection may result as much from activation of inhibitory CD8+ , IL-10-secreting T cells which are specific for OFA or TSTA as from poor immunogenicity of the emerging host tumor (30, 31). TSTA-specific inhibitory T cell clones have not been detected to date, suggesting that OFA-specific TS cells secreting IL-10 serve a regulatory role in inhibiting both OFA and TSTA-specific TC cytotoxicity in vitro and possibly in vivo (30, 31). All of the iLRP-reactive clones responded by proliferation and cytokine production to purified OFA in a manner identical to their response to iLRP. Since tumor cells often reduce the amount of class 1 MHC proteins made, it has been suggested that any vaccine should include both peptides that can induce TH1 cells and TC cells (53). This is because activation of TH1 cells will allow antigen-presenting cells to be utilized to activate immature TC and that once activated less class 1 is required for allowing target recognition and killing than for activation of naive TC (55). While not intending to be bound by any particular theory of operation, Applicants believe that intact iLRP has epitopes capable of inducing both types of T cells.

### REFERENCES II

1. Coggin, J. H., Jr., K. R. Ambrose, and N. G. Anderson. 1970. Fetal antigen capable of inducing transplantation immunity against SV40 tumors. J. Immunol. 105:524.
2. Coggin, J. H., Jr., K. R. Ambrose, B. B. Bellomy, and N. G. Anderson. 1971. Tumor immunity in hamsters immunized with fetal tissues. J. Immunol. 107:526.
3. Payne, W. J., Jr. and J. H. Coggin, Jr. 1985. Mouse monoclonal antibody to embryonic antigen: Development, cross-reactivity with rodent and human tumors, and preliminary polypeptide characterization. J. Natl. Cancer Inst. 75:527.
4. Coggin, J. H., Jr., S. D. Rohrer, E. D. Leinbach, R. B. Hester, P. I. Liu, and L. S. Heath. 1988. Radiation-induced lymphoblastic lymphomas/leukemias and sarcomas of mice express conserved, immunogenic 44 kilodalton oncofetal antigen. Am. J. Pathol. 130:136.
5. Coggin, J. H., Jr., L. Adkison, and N. G. Anderson. 1980. Fetal antigens shared as transplantation rejection antigens on chemically induced mouse and hamster sarcomas. Cancer Res. 40:1568.
6. Ambrose, K. R., N. G. Anderson, and J. H. Coggin, Jr. 1971. Interruption of SV40 oncogenenesis with human fetal antigen. Nature 233:194.
7. Leffel, M. S. and J. H. Coggin, Jr. 1977. Common transplantation antigens on 3-MCA-induced murine sarcomas detected by three methods of assays. Cancer Res. 37:4112.
8. Hellstrom, K. E. and I. Hellstrom. 1975. Studies on the mechanisms of tumor immunity to common, possibly embryonic antigens in mouse sarcomas. In: Fundamental Aspects of Neoplasia. A. A. Gotlieb, O. J. Plescia, and D. H. L. Bishop, eds. Springer-Verlag, New York, p. 89.
9. Coggin, J. H., Jr. and K. R. Ambrose. 1979. Embryonic and fetal determinants on virally and chemically-induced tumors. In: Methods in Cancer Research. W. H. Fishman and H. Busch, eds. Academic Press, New York, p. 18.
10. Gussack, G. S., S. D. Rohrer, R. B. Hester, P. I. Liu, and J. H. Coggin, Jr. 1988. Human squamous cell carcinoma lines express oncofetal antigen 44 kilodalton polypeptide defined by monoclonal antibody to mouse fetus. Cancer 62:57.
11. Coggin, J. H., Jr. 1994. Classification of tumor associated antigens in rodents and humans. Immunol. Today 15:246.
12. Ambrose, K. R., N. G. Anderson, and J. H. Coggin, Jr. 1971. Cytostatic activity and SV40 tumor immunity in hamsters. Nature 233:321.
13. Rohrer, J. W., C. Culpepper, A. L. Barsoum, and J. H. Coggin, Jr. 1995. Characterization of RFM mouse T lymphocyte anti-OFA immunity in apparent tumor-free, long-term survivors of sublethal X-irradiation by limiting dilution T lymphocyte cloning. J. Immunol. 154:2266.
14. Coggin, J. H., Jr. 1986. Oncofetal antigens. Nature 319:428.
15. Girardi, A. J., P. Reppuci, P. Dierlman, W. Rutala, and J. H. Coggin, Jr. 1973. Prevention of Simian Virus 40 tumors by hamster fetal tissue: Influence of parity status of donor females on immunogenicity of fetal tissue and immune cell cytotoxicity. Proc. Natl. Acad. Sci. (USA) 70:183.
16. Barsoum, A. L. and J. H. Coggin, Jr. 1991. Isolation and partial characterization of a soluble OFA from murine and human amniotic fluids. Int. J. Cancer 48:248.
17. Coggin, J., S. Rohrer, R. Hester, A. Barsoum, H. Rashid, and G. Gussack. 1993. 44-kD oncofetal transplantation antigen in rodent and human fetal cells: Implications of recrudescence in human and rodent cancers. Arch. Otolaryngol. Head and Neck Surgery 119:1257.
18. Rohrer, S. D., R. N. Sarli, A. L. Barsoum, R. B. Hester, and J. H. Coggin, Jr. 1992. Expression of 44 kilodalton oncofetal antigen as a premalignancy marker in X-irradiation-induced murine T-cell lymphoma. J. Natl. Cancer Inst. 84:602.
19. Barsoum, A. L. and J. H. Coggin, Jr. 1989. Immunogenicity of a soluble, partially purified oncofetal antigen from murine fibrosarcoma in syngeneic mice. J. Biol. Resp. Modifiers 8:579.
20. Rohrer, J. W., S. D. Rohrer, A. Barsoum, and J. H. Coggin, Jr. 1994. Differential recognition of murine tumor-associated oncofetal transplantation antigen and individually specific tumor transplantation antigens by syngeneic cloned BALB/c and RFM mouse T cells. J. Immunol. 152:754.
21. Menard, S., V. Castronovo, E. Tagliabue, and M. E. Sobel. 1997. New insights into the metastasis-associated 67 kD laminin receptor. J. Cell Biochem. 67:155.
22. Landowski, T. H., E. A. Dratz, and J. R. Starkey. 1995. Studies of the structure of the metastasis-associated 67 kDa laminin binding protein: Fatty acid acylation and evidence supporting dimerization of the 32 kDa gene product to form the mature protein. Biochem. 34:11276.
23. Castronovo, V., F. A. van den Brule, P. Jackers, N. Clausse, F. T. Liu, C. Gillet, and M. E. Sobel. 1996. Decreased expression of galectin-3 is associated with progression of human breast cancer. J. Pathol. 179:43.
24. Van den Brule, F. A., C. Cuicu, A. Berchuck, R. C. Bast, M. Deprez, F. T. Liu, D. N. Cooper, C. Pieters, M. E. Sobel, and V. Castronovo. 1996. Expression of the 67-kd laminin receptor, galectin-1, and galectin-3 in advanced human uterine adenocarcinoma. Hum. Pathol. 27:1185.
25. Mafune, K., T. S. Ravikumar, J. M. Wang, H. Yow, L. B. Chen, and G. D. Steele, Jr. 1990. Expression of Mr 32,000 laminin-binding protein messenger RNA in human colon carcinoma correlates with disease progression. Cancer Res. 50:3888.
26. Sobel, M. E. 1993. Differential expression of the 67 kDa laminin receptor in cancer. Cancer Biol. 4:311.
27. Barsoum, A. L., J. Kirk, R. Keith, J. P. Houchins, J. W. Rohrer, J. Weatherbee, and J. H. Coggin, Jr. 1998. Sequence homology between 44 kD OFA kD oncofetal antigen and high affinity immature laminin receptor protein. Proc. Am. Assoc. Can. Res. 39:A1809.
28. Van den Brule, F. A., J. Price, M. E. Sobel, R. Lambotte, and V. Castronovo. 1994. Inverse expression of the laminin-binding proteins, 67LR and galectin-3, correlates with the invasive phenotype of trophoblastic tissue. Biochem. Biophys. Res. Commun. 201:388.
29. Castronovo, V. 1993. Laminin receptors and laminin-binding proteins during tumor invasion and metastasis. Invasion Metastasis 13:1.
30. Coggin, J. H., Jr., A. L. Barsoum, and J. W. Rohrer. 1998. Tumors express both unique TSTA and cross-protective 44 kDa oncofetal antigen. Immunol. Today 19:405, 1998.
31. Coggin, J. H., Jr., J. W. Rohrer, and A. L. Barsoum. 1997. A new immunobiological view of radiation-promoted lymphomagenesis. Int. J. Radiat. Biol. 71:81.
32. Rabin, H., R. F. Hopkins III, F. W. Ruscetti, R. H. Neubauer, R. L. Brown, and T. G. Kawakami. 1981. Spontaneous release of a factor with the properties of T cell growth factor from a continuous line of primate tumor T cells. J. Immunol. 127:1852.
33. Abou-Zeid, C., E. Filley, J. Steele, and G. A. W. Rook. 1987. A simple new method for using antigens separated by polyacrylamide gel electrophoresis to stimulate lymphocytes in vitro after converting bands cut from Western blots into antigen-bearing particles. J. Immunol. Methods 98:5.
34. Laemmli, U. K. 1970. Cleavage of structural proteins during the assembly of the head of bacteriophage T4. Nature 227:68. 35. Towbin, H., T. Staehlin, and J. Gordon. 1979. Electrophoretic transfer of proteins from polyacrylamide gels to nitrocellulose sheets: Procedure and some applications. Proc. Natl. Acad. Sci. (USA) 76:4350.
36. Harlow, E. and L. Lome. 1988. Antibodies: A Laboratory Manual. Cold Spring Harbor Laboratories, NY, p. 494.
37. Redinbaugh, M. G. and R. B. Turley. 1986. Adaptation of the bicinchoninic acid protein assay for use with microtiter plates and sucrose gradient fractions. Anal. Biochem. 153:267.
38. Mayfield, J. E., D. J. Bricker, H. Godfrey, R. M. Crosby, D. J. Knight, S. M. Halling, D. Balinsky, and L. B. Tabatabai. 1988. The cloning, expression, and nucleotide sequence of a gene coding for an immunogenic Brucella abortus protein. Gene 63:1.
39. Takayama, S., T. Sato, S. Krajewski, K. Kochel, S. Irie, J. A. Millan, and J. C. Reed. 1995. Cloning and functional analysis of BAG-1: a novel Bcl-2-binding protein with anti-cell death activity. Cell 80:279.
40. Goding, J. W. 1980. Antibody production by hybridomas. J. Immunol. Methods 39:285.
41. Gajewski, T. F., and F. W. Fitch. 1988. Antiproliferative effect of IFN-gamma in immune regulation I. IFN-γ inhibits the proliferation of TH2 but not TH1 murine helper T lymphocyte clones. J. Immunol. 140:4245.
42. Rogers, L. A., A. Zlotnik, F. Lee, and K. Shortman. 1991. The maintenance of lytic specificity during the development of clones of cytotoxic T lymphocytes from single precursor cells. J. Immunol. Methods 143:241.
43. Decker, T. and M.-L. Lohmann-Mathes. 1988. A quick and simple method for the quantitation of lactate dehydrogenase release in measurements of cellular cytotoxicity and tumor necrosis factor (TNF) activity. J. Immunol. Methods 15:61.
44. Rohrer, J. W. and J. H. Coggin, Jr. 1995. CD8 T cell clones inhibit antitumor T cell function by secreting IL-10. J. Immunol. 155:5719.
45. Srvistava, P. S. and L. J. Old. 1988. Individually distinct transplantation antigens on chemically-induced mouse tumors. Immunol. Today 9:78.
46. Hellstrom, K. E. an I. Hellstrom. 1967. Cell bound-immunity to autologous and syngeneic mouse tumors induced by methylcholanthrene and plastic discs. Science 156:981.
47. DeLeo, A. B., H. Shikn, T. Takahashi, M. John, and L. J. Old. 1977. Cell surface antigens on chemically-induced sarcomas of the mouse. I. Murine leukemia virus-related antigens and alloantigens of cultured fibroblasts and sarcoma cells: Description of a unique antigen on BALB/c MethA sarcoma. J. Exp. Med. 146:720.
48. Coggin, J. H., Jr. 1989. Cross-reacting tumor-associated transplantation antigen on primary 3-methylcholanthrene-induced BALB/c sarcomas. Mol. Biother. 1:223.
49. Van den Eynde, B. J. and P. van der Bruggen. 1997. T cell defined tumor antigens. Curr. Opinion in Immunol. 9:684.
50. Melief, C. and W. Kast. 1995. T-cell immunotherapy of tumors by adoptive transfer of cytotoxic T lymphocytes and by vaccination with minimal essential epitopes. Immunol. Rev. 145:167.
51. Breenberg, P. D. 1991. Adoptive t cell therapy of tumors: Mechanisms operative in the recognition and elimination of tumor cells. Adv. Immunol. 49-281.
52. Coulie, P. G., P. Weynants, G. Lehmann, J. Herman, V. Brichard, T. Walfel, A. Van Pel, E. De Plaen, F. Brasseur, and T. Boon. 1993. Genes coding for tumor antigens recognized by human cytolytic T lymphocytes. J. Immunother. 14:104.
53. Hellstrom, I. and K. E. Hellstrom. 1998. T cell immunity to tumor antigens. Crit. Rev. in Immunol. 18:1.
54. Tsang, K. Y., S. Zaremba, C. A. Nieroda, M. Z. Zhu, J. M. Hamilton, and J. Schlom. 1995. Generation of human cytotoxic T cells specific for human carcinoembryonic antigen epitopes from patients immunized with recombinant vaccinia-CEA vaccine. J. Natl. Cancer Inst. 87:982.
55. Hellstrom, I., P. Gladstone, and K. Hellstrom. 1997. Cancer vaccines: Challenges and potential solutions. Molec. Med. Today 3:286.
56. Kawakami, Y. and S. A. Rosenberg. 1997. Human tumor antigens recognized by T cells. Immunol. Res. 16:313.
57. Boon, T. and L. J. Old. 1997. Cancer tumor antigens: Editorial overview. Curr. Opinion in Immunol. 9:681.
58. Velders, M. P., J. D. Nieland, M. P. Rudolf, K. Loviscek, S. Weijzen, K. E. de Visser, M. Fatima Macedo, M. Carbone, and W. M. Kast. 1998. Identification of peptides for immunotherapy of cancer. It is still worth the effort. Crit. Rev. in Immunol. 18:7.
59. Restifo, N. P., R. Esquivel, A. C. Asher, H. Stotter, R. J. Barth, J. R. Bennick, J. J. Mule, J. W. Yewdell, and S. A. Rosenberg. 1991. Defective presentation of endogenous antigens by a murine sarcoma:Implications for the failure of anti-tumor response. J. Immunol. 147:1453.
60. Finke, J. H., A. H. Zea, J. Stanley, C. L. Longo, H. Mizoguchi, R. R. Tubbs, R. H. Wiltrout, J. J. O'Shea, S. Kudoh, E. Klein, R. M. Bukowski, and A. C. Ochoa. 1993. Loss of T cell receptor chain and p561ck in T-cells infiltrating human renal cell carcinoma. Cancer Res. 53:5613.
61. Bost, K. L., S. C. Bieglik, and B. M. Jaffe. 1995. Lymphokine mRNA expression by transplantable murine B lymphocyte malignancies:Tumor-derived IL-10 as a possible mechanism for modulating the anti-tumor response. J. Immunol. 154:718.
62. Kudoh, S., C. Redovan, P. Raymann, M. Edinger, R. R. Tubbs, A. Novick, J. H. Finke, and R. M. Bukowski. 1997. Defective granzyme B gene expression and lytic response in T lymphocytes infiltrating human renal cell carcinoma. J. Immunother. 20:479.
63. Schild, H., M. Norda, K. Deres, K. Falk, O. Rotzschke, K. H. Weismuller, G. Jung, and H. G. Rammensee. 1991. Fine specificity of cytotoxic T lymphocytes primed in vivo either with virus or synthetic lipopeptide vaccine or primed in vitro with peptide. J. Exp. Med. 174:1665.
64. Toes, R. E. M., E. I. H. van der Voort, S. P. Schoenberger, J. Wouter Drijfhout, L. van Bloois, G. Storm, W. M. Kast, R. Offringa, and C. J. M. Melief. 1998. Enhancement of tumor outgrowth through CTL tolerization after peptide vaccination is avoided by peptide presentation on dendritic cells. J. Immunol. 160:4449.

### EXAMPLE 30

Immature Laminin Receptor Protein as an Oncofetal Antigen

Sequence Homology and Cross-Reactivity of the 44 kD Oncofetal Antigen with the High Affinity Immature Laminin Receptor Protein.

In this example, amino acid sequence of two peptides of OFA showed complete homology with “immature” laminin receptor protein (iLRP). The cDNA encoding iLRP was cloned from a 7 day gestation mouse fetal cDNA library, and the protein was expressed in E. coli. Anti-44 kD OFA mAbs, which bound specifically to native 44 kD OFA in ELISA and Flow Cytometric (FC) analyses of human and rodent tumor and fetal cells, cross-reacted with recombinant iLRP. In a concentration-dependent manner, recombinant iLRP blocked the binding of these anti-44 kD OFA mAbs to OFA⁺ tumor cells in FC analysis and to purified labeled OFA in an ELISA. A new panel of IgG anti-iLRP mAbs was generated by immunization of BALB/c mice with purified, recombinant iLRP. One of these anti-iLRP mAbs was used to purify the native 44 kD protein (P44), which is recognized by OFA specific mAbs, from an X-ray induced T-cell lymphoma of RFM mice. MALDI-TOF mass spectrometry of trypsin digested P44 revealed proteolytic fragments, which covered 67% of the sequence length that were entirely consistent with the predicted iLRP a.a. sequence. These results demonstrate that immunogenic 44 kD OFA is closely related or identical to an immunogenic form of iLRP restricted to early embryo and tumor cells.

Materials

All chemicals, unless otherwise stated, were purchased from Sigma Chemical Co. (St. Louis, Mo.). Biotinyl-E-aminocaproic acid N-hydroxysuccinimide ester and Vectastain horseradish peroxidase kits were obtained from Vector Laboratories (Burlingame, Calif.). Nitrocellulose sheets (BA-85, 0.45 μm pore size) were purchased from Schleicher and Schuell (Keene, N.H.).

Monoclonal Antibodies

Anti-OFA IgM monoclonal antibodies (38.46, 38.7, 69.1 and 115) used in these experiments were generated by syngeneic immunization with midgestational fetal cells as described previously (6). The hybridomas were grown in serum-free medium and culture supernatant collected, concentrated by ultrafiltration and fractionated on a Sephacryl S-300 HR. The high molecular weight peak exhibiting IgM activity was collected and adjusted to 1 mg/ml by ultrafiltration. Anti-LRP antibodies were generated by immunization of a BALB/c mouse with recombinant iLRP.

Preparation of IgM mAb 115 Matrix

For the affinity purification of OFA, anti-OFA mAb115 (10 mg, 3 mg/ml) in 0.2 M NaHCO₃, 0.5 M NaCl, pH 8.3, was coupled to 1 ml of N-hydroxysuccinimide activated Sepharose 4 fast flow (Pharmacia, Piscataway, N.J.) overnight at 10° C. according to the recommendations of the supplier. The coupling yield was 95%.

Recombinant iLRP

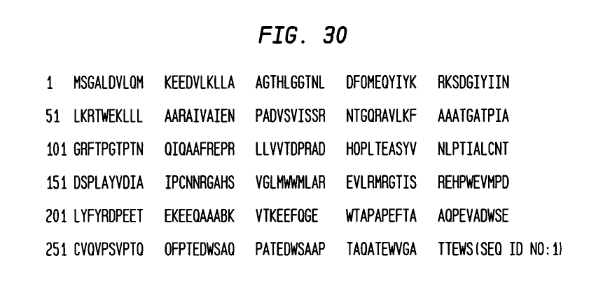

Oligonucleotides corresponding to sequenced amino acid segments (FIG. 30) were used to probe a 7-ay Swiss/Webster mouse embryo library purchased from Clontech (Palo Alto, Calif.). A full-length cDNA was identified and was cloned into an expression vector under the control of the tac promoter, and the protein was expressed in E. coli. Inclusion bodies were isolated and solubilized in 6M guanidine hydrochloride in 20 mM Tris pH8.0, 0.1N NaCl, 2 mM EDTA, 0.2% sodium azide. The solubilized protein was added to six volumes of 20 mM tris pH 8.0, 1 M guanidine HCl, 2 mM reduced glutathione, 0.2 mM oxidized glutathione, was renatured for 18 hours at 4° C., and then dialyzed against 20 mM tris pH 8.0, 0.1 M NaCl, 0.04% azide.

Internal Amino Acid Sequencing

(A) 44 kD OFA. Plasma membrane fractions from murine XR11-5T cell line (7), propagated subcutaneously in syngeneic RFM mice, were prepared as described previously (25). The membrane pellet was solubilized in 2% n-octylglucoside in 10 mM NaN₃, 10 mM iodoacetamide and 10 mM Tris-HCl, pH 8.0, containing the protease inhibitors: aprotinin (100 KIU/ml), phenylmethylsulfonyl fluoride (PMSF, 1 μM), leupeptin (15 μM), N-α-p-tosyl-L-lysine chloromethyl ketone (TLCK, 50 μM) and soybean typsin inhibitor (5 μg/ml). Solubilized membranes were centrifuged at 100,000 g for 1-hr to remove insoluble material. The membrane extract was passed first through a monoQ-column equilibrated with 0.05 M Tris-HCl, pH 8, and bound proteins eluted with a linear salt gradient (0.05 M Tris-HCl: 0.05 M Tris-HCl+1 M NaCl, pH 8) as described previously (26). The peak containing OFA activity was collected and incubated with mAb115-affinity beads. After thorough washing of the column with Tris-saline buffer, pH 7.4 containing 0.05% Tween-20, antibody bound material was eluted by heating the Sepharose beads in 1 volume of reducing SDS-PAGE sample buffer at 95° C. for 10 min. Six equal aliquots were placed in the wells of a 12% polyacrylamide gel and electrophoresis was performed using a Tris-Tricine-SDS running buffer system according to Laemmli (27). For obtaining internal a.a. sequences from SDS PAGE-separated protein, 44 kD Coomassie blue stained bands were carefully cut out with a clean scalpel for “In gel” tryptic digestion, preparative HPLC with peak detection and collection, and NH₂-terminal sequencing of the separated peptides using standard procedures (28,29). Computer searches of the protein databases for identity or similarity of the identified sequence with known proteins was carried out with the FASTA computer program of GCG.

(B) P44. P44 was isolated from a cytoplasmic extract of the murine XR11-5T cells. The extract was subjected to affinity chromatography using anti-LRP IgG mAb 43532, run on SDS-PAGE, and transferred to nitrocellulose. Sequencing and mass spectral analysis was carried out at the Microsequencing Laboratory of the Worchester Foundation for Biomedical Research (Shrewsbury, Mass.). Following trypsin digestion and isolation of peptides on reverse phase HPLC, peptides were analyzed by MALDI-TOF mass spectrometry to determine purity and size. One peptide was selected for sequencing.

Flow Cytometry Analysis

MCA-1315 cells grown in culture were harvested by treatment with PBS-EDTA, washed in staining buffer (PBS, pH 7.4, 2% BSA, and 0.1 sodium azide) and aliquoted at 2.5×10⁴ cells/sample. Cells were incubated with the appropriate dilution of anti-OFA mAb in staining buffer, either alone or with 1-5 μg of iLRP for 1 h at 4° C. An aliquot of cells was also stained with control mouse IgM (MOPC-104E) at the same dilution (10 μg/ml). The excess primary Ab was removed by washing, and FITC-labeled goat anti-mouse IgM (Organon Teknika Corp., West Chester, Pa.) was added for 30 min at 4° C. as second reagent for indirect immunofluorescent staining. Flow cytometry was performed using a FACS 440 (Becton Dickinson, San Jose, Calif.) equipped with WinMDI software. Flow cytometry data are depicted as histograms of cell number (y-axis) vs. fluorescence intensity (x-axis) on a log scale from representative experiments.

SDS-PAGE and Western Blot

Purified iLRP in SDS buffer, heated for 5 min at 95° C., was electrophoresed on 12% polyacrylamide gel (0.75 μg/lane) under reducing conditions and the proteins were transferred onto Immobilon polyvinylidene difluoride membranes (Millipore, Bedford, Mass. ) in 48 mM Tris and 39 mM glycine (20% methanol), pH 9.2, using a wet-type electroblotter for 60 min at 600 mA (30). The membranes were blocked overnight at 4° C. with Tris-buffered saline, 0.1% Tween-20, and 5% powdered milk and probed with either mAb 38.46, 38.7, 69.1 or 115, followed by biotinylated goat anti-mouse μ-chain specific antibody (diluted to 1:5000 v/v in blocking buffer) and the AB complex (Vector Labs, Burlingame, Calif.) according to the manufacturer's recommendation. The peroxidase reaction was initiated by using 4 chloro-naphthol and H₂O₂ in TBS, and was stopped by washing in water.

Biotinylation of OFA

Purified OFA was obtained as described for internal aa sequence of 44 kD OFA, except that the OFA was eluted with 0.1 M glycine-HCl, 0.5 m NaCl pH 2.7 adjusted to pH 8 with Tris-base, dialysed against 0.1 M NaHCO₃ and used for biotinylation. Purified OFA was conjugated to biotin by the succinimide ester method as described previously (31). Briefly, the protein was dialysed overnight against 0.1 M NaHCO₃, pH 8. The biotinsuccinimide ester was dissolved in dimethyl formamide at 1 mg/ml immediately before use, added to the protein at a ratio of 1:10 (mol/mol) and mixed immediately. The mixture was incubated at room temperature for 4 hr and then dialysed for 36 hours against PBS containing 0.1% sodium azide with several changes, and stored at 4° C.

ELISA

(A) Direct ELISA assay. Flat-bottomed 96-well plates were coated with iLRP 300 ng/100 μl/well and post coated with 1% BSA in PBS, pH 7.2. A direct binding curve for anti-OFA mAb was generated by incubating 100 μl of a serial dilution of the antibodies (original adjusted to 1 mg/ml) in 0.5% BSA in PBS at 37° C. for 1 hr. The plate was washed four times for 5 min each with PBS-T solution (PBS containing 0.05% Tween-20). The plate was further incubated with a biotinylated goat anti-mouse μ-chain specific antibody at 1:5000 dilution in 0.5% BSA in PBS for 1 hr. The plate was washed again as described previously and 100 μl of an AB reagent (avidin:biotinylated horseradish peroxidase, Vector Laboratories; one drop of each in 10 ml PBS-T) were added to each well of the microplate and incubated for 30 min at room temperature. The plate was washed as described previously. Finally, 100 μl of the substrate solution (ABTS: 2,2′-azino-di-(3-ethylbenz-thiazoline sulfonate) in 0.1 citrate buffer, pH 4 were added to each well. After an incubation period of 30 min at room temperature, the colored product was measured spectrophotometrically at 410 nm in a microELISA reader. The tests were done in triplicate.

(B) Competitive ELISA. In Competitive ELISA the 96-well plates were coated with goat anti-mouse IgM (250 ng/100 μl/well) and blocked with 1% BSA in PBS, pH 7.2. The plate was washed four times for 5 min each with PBS-T solution (PBS containing 0.05% Tween-20). The plate was further incubated with 100 μl of either one of the following IgM mAbs (38.46, 38.7, 69.1, or 115) at 5 μg/ml for 1 hr at 37° C. The plate was again washed four times as described previously. 50 μl of biotinylated purified OFA, predetermined to give an optimal reading were incubated for 16 hours at 10° C. together with increasing quantities (31 ng to 2 μg) of cold iLRP. The plate was washed again as described previously and 100 μl of the AB complex were added to each well of the microplate and incubated for 30 min at room temp. After washing the plate-an additional four times, 100 μl of the ABTS solution in 0.1 citrate puffer, pH 4 were added to each well. After an incubation period of 30 min, the colored product was measured spectrophotometrically at 405 nm in a microELISA reader. The tests were done in triplicate. The percent inhibition was calculated from the formula:

[1−(OD reading of Ab before absorption−OD reading of background)/OD reading after absorption−OD reading of background)]×100. Experimental values are presented as the mean±S.E.M. of the number of individual assays.

Amino Acid Sequence of OFA and P44 Peptides are Identical to those of iLRP

OFA. Sequence data obtained on one of the peaks from the lysyl endopeptidase digest of the purified 44 kD OFA revealed 2 peptides: a primary peptide consisting of 23 aa and a secondary peptide of 10 aa. The sequences of the peptides were LLAAGTHLGGTNLDFQMEQYIYK (residues 18-40) and SDGIYIINLK (residues 43-52) respectively (FIG. 30 (SEQ ID NO:1), underlined letters). Protein data searches showed that the sequence of both peptides matched the “metastasis-associated 67 kD high affinity laminin receptor” protein from several species (15,16).

(B) P44. Peptide-digest from P44 was fractionated on reverse phase, HPLC and fractions analyzed by MALDI-TOF mass spectrometry to determine purity and size. All of the peptide masses equaled predicted masses of peptide fragments that would be obtained by trypsin digestion of the previously described iLRP. Portions of the protein for which corresponding peptides were identified are shaded in FIG. 30. One fraction was selected for sequencing, and the sequence obtained was identical to a.a. residues 64-80 within laminin receptor protein precursor. The sequenced segment is shown in bold (FIG. 30).

Anti-OFA mAbs Bind to iLRP

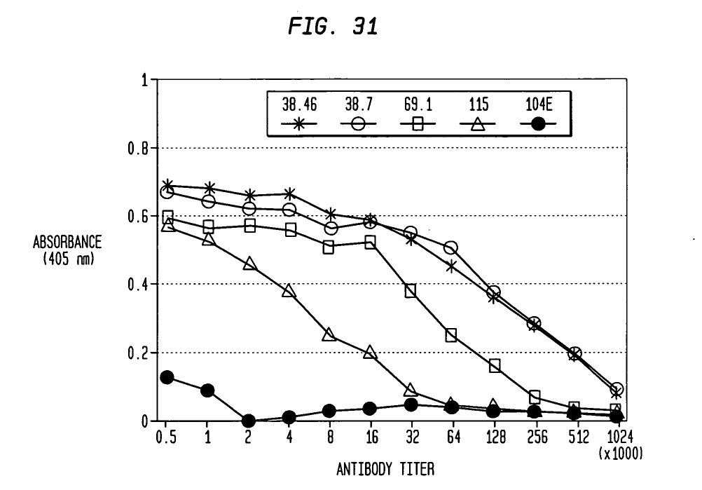

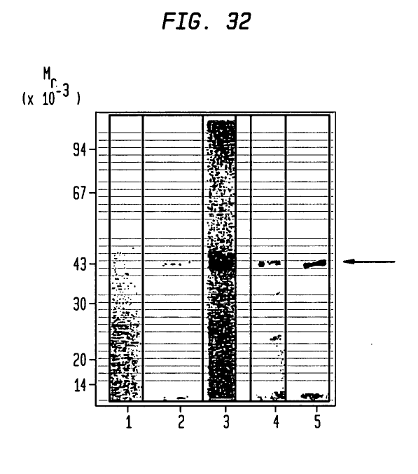

The binding of four anti-OFA IgM mAbs (38.46, 38.7, 69.1 and 115) to purified iLRP was checked in a direct ELISA assay. All anti-OFA mAb tested showed significant binding to iLRP in a concentration dependent fashion (FIG. 31) with 38.46 and 38.7 showing the highest (1/512 000 of the original concentration, equivalent to 1.95 ng/ml), 69.1 a moderate (1/128 000, equivalent to 7.8 ng/ml) and 115 the lowest binding titer (1/16 000, equivalent to 62.5 ng/ml).

To confirm that these mAbs bound to iLRP and not to a minor bacterial contaminant in the preparation of the iLRP, we performed an SDS-PAGE on the iLRP and prepared a western blot. When the blots were stained with the anti-OFA mAbs, all four antibodies gave only one band of the molecular size anticipated for iLRP (FIG. 32).

iLRP Inhibits the Binding of Anti-OFA mAbs to OFA

The specificity of binding of four mAbs (38.46, 38.7, 69.1 and 115) to purified iLRP was further checked by competitive ELISA, and by competitive Flow cytometry.

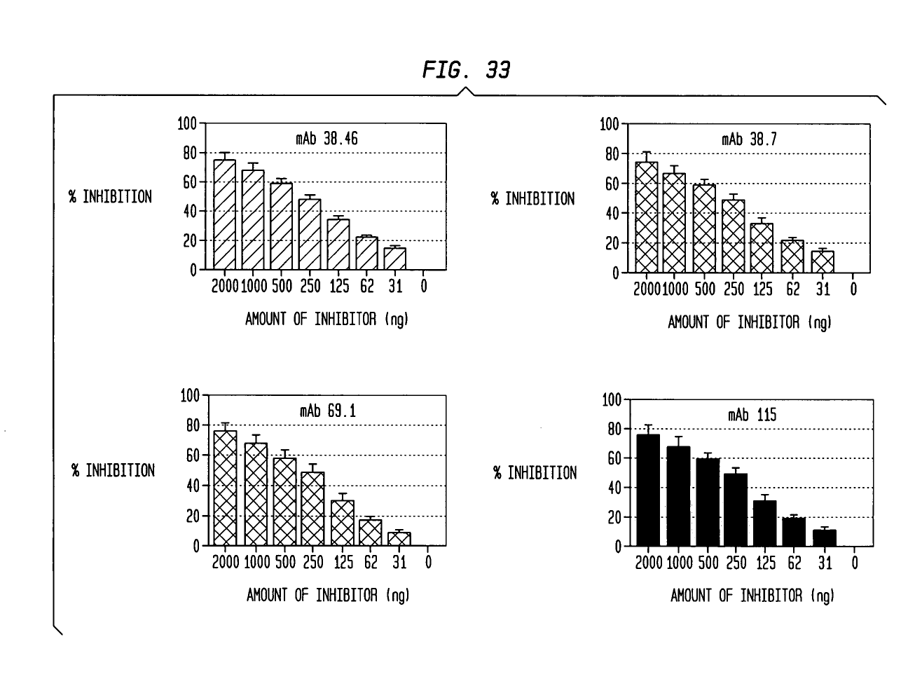

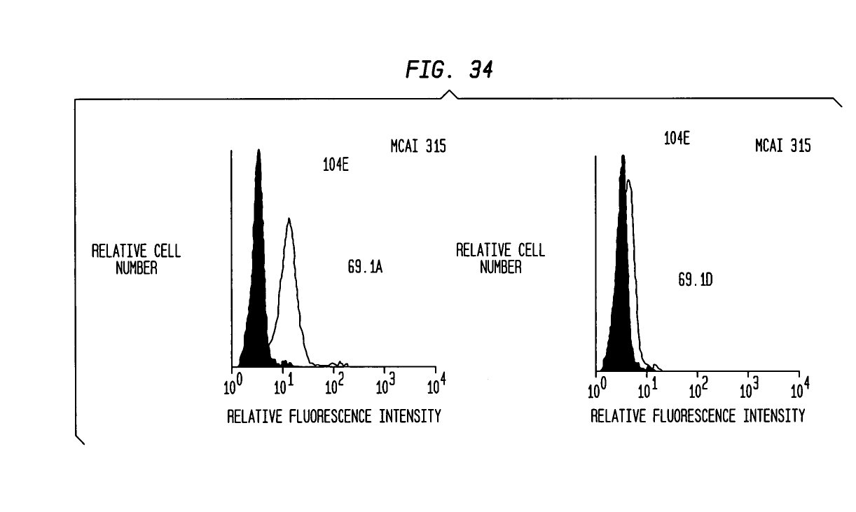

A. Competitive ELISA. In the competitive ELISA, graded amounts of iLRP (31 ng to 2 μg) were used to compete with a predetermined amount of biotinylated OFA for binding to anti-OFA IgM mAbs (38.46, 38.7, 69.1 and 115). With all four mAbs, iLRP could compete effectively in a concentration dependent fashion with OFA for binding to a fixed amount of anti-OFA antibodies bound to the surface of the microwells through an anti-mouse IgM antibody (FIG. 33). B. Competitive flow cytometry. Direct binding flow cytometry assays of the anti-OFA mAbs 38.46, 38.7 69.1 and 115 to several tumor cell types reveal that staining is strongest with 69.1, moderate with 115 and weak with both 38.46 and 38.7 under the same experimental conditions (data not shown). We chose mAb 69.1 to test if addition of iLRP to the antibody would compete with 44 kD OFA on the surface of MCA1315 BALB/c fibrosarcoma cells. Different graded amounts of iLRP (1 μg-5 μg) competed effectively with cell surface OFA, with 5 μg showing the highest competition (data shown for 5 μg only, FIG. 34).

Discussion

The results reported here show several shared characteristics of OFA and iLRP. A. A. sequences of two peptides from affinity purified 44 kD OFA were identical to that of 32 kD iLRP from several species (residues 18-40 and 43-52, underlined in FIG. 30). An IgG monoclonal antibody generated against iLRP was used to purify a native, 44 kD protein (P44) from murine XR11-5T cells. This purified protein reacts with OFA-specific IgMs (data not shown) and internal a.a. sequence of one of the peptides was identical to iLRP (residues 64-80, shown in bold in FIG. 30) and confirmed the same a.a. sequence reported for the peptides detected in 44 kD OFA isolated from mAb 115-purified protein from XR11-5T cells. MALDI-TOF mass spectrometry of trypsin digested P44 revealed proteolytic fragments covered 67% of the sequence length that were entirely consistent with the predicted iLRP a.a. sequence. The epitope for several mAbs to OFA has been localized within 25 a.a. at the C-terminal end of iLRP (data not shown). This finding indicates that identical sequence for the B cell epitope(s) in the unsequenced portion of the iLRP matches sequence in the 32% of the remaining unsequenced 44 kD OFA. Moreover, direct antibody binding ELISA assays show significant binding of all the four anti-OFA antibodies to iLRP (FIG. 31), and the antibodies detect one band at an approximate molecular weight of about 44 kD in the western blot (FIG. 32). This binding could be specifically inhibited by competition between iLRP and labeled OFA (competitive ELISA, FIG. 33) or by competition between soluble iLRP and cell surface OFA on MCA1315 cells (inhibition flow cytometry, FIG. 34).

OFA and iLRP share several other properties. For example, OFA is immunologically conserved in mammals (3-5,32,33). The nucleotide sequence of the iLRP gene is strongly conserved in birds and mammals (14).

OFA is developmentally regulated. The expression of the 44-kD OFA in the fetus is stage specific. It appears shortly after gestation, peaks at midgestation and then falls gradually thereafter to non-detectable levels (8). The expression of the 67LR, a product of the iLRP gene, was quantitated in human trophoblastic specimens at different gestational ages using Northern and Western blot techniques. Expression of the 67LR in humans was found to increase starting at fetal age 7 weeks and reach a maximum at 12 weeks, when invasion is maximal, and then to decrease (34).

OFA protein has been detected in carcinomas, lymphomas and sarcomas, but not in a vast survey of normal tissues from humans and rodents or in normal patient autologous tissues where available, using flow cytometry and immunoprecipitation with Coomassie staining (6,7,35). 67 kD laminin receptor is present on the surfaces of both normal and malignant cells, but overexpressed on the cancer cell surface. Overexpression of the 67 LR correlates with proliferation (36) as well as the invasive and metastatic capacity (37) of the cancer cells. This overexpression can be attributed to overproduction of 32 kD iLRP in the cancer cell, since transfection of Chinese hamster ovary (CHO) with the hamster iLRP gene in pcDNA/neo vector resulted in overexpression of 67 LRP (38). 67LR is overexpressed not only in epithelial tumors but also in melanomas and lymphomas [reviewed in 14].

OFA is immunogenic in the syngeneic host (3,10,32,39,40). Recently it has been reported (11-13) that 44 kD OFA is a significant T-cell immunogen arousing both CD4 and CD8 cytotoxic [TC] and non-cytotoxic inhibitory [TS] cells in mice. In another report, it was shown that recombinant iLRP, purified P44 and 44 kD OFA restimulate OFA-specific T-cell subclasses in vitro, originally stimulated in vivo by primary murine XR11-4T and XR11-5T cells expressing 44 kD OFA (J. W. Rohrer et al.: Manuscript in preparation). On the other side, an association between iLRP overexpression and the presence of ãä T-cells in human lung cancer was recently reported (41,42). There is also a relationship between the levels of laminin receptor expression on cultured lung cancer cells and their susceptibility to specific lysis by ãä+, but not áâ+, TILs. This specific cell killing was not T-cell receptor mediated, but it was inhibited by addition of the anti-67 LR mAb MLuC5 and by a synthetic peptide of the laminin A chain (43).

Immunogenic 44 kD OFA is closely related to the immature LRP. They are considered as equivalents for purposes of the present invention. This protein is overexpressed on malignant rodent and fetal cells and on human cancer and fetal cells and is associated with invasiveness.

### REFERENCES III

1. Ambrose, K. R., N. G. Anderson, and J. H. Coggin 1971. Interruption of SV40 oncogenesis with human fetal antigen. Nature (Lond) 233:194.
2. Coggin, J. H., Jr., and K. R. Ambrose. 1979. Embryonic and fetal determinants on virally and chemically induced tumors. Methods Cancer Res 18:371.
3. Coggin, J. H., K. R. Ambrose, and N. G. Anderson. 1970. Fetal antigen capable of inducing transplantation immunity against SV40 hamster tumor cells. J. Immunol. 105:524.
4. Coggin, J. H., Jr., L. Adkinson, and N. G. Anderson. 1980. Fetal antigens shared as transplantation rejection antigens on chemically induced mouse and hamster sarcomas. Cancer Res. 40:1568.
5. Coggin, J. H., Jr., and N. G. Anderson. 1974. Cancer, differentiation, and embryonic antigens: Some Central Problems. Adv. Cancer Res. 19:105.
6. Payne, W. J., Jr., and J. H. Coggin, Jr. 1985. Mouse monoclonal antibody to embryonic antigen: development, cross-reactivity with rodent and human tumors, and preliminary polypeptide characterization. J. Natl. Cancer Inst. 75:527.
7. Coggin, J. H., Jr., S. D. Rohrer, E. D. Leinbach, R. B. Hester, P. I. Liu, and L. S. Heath. 1988. Radiation-induced lymphoblastic lymphomas\leukemias and sarcomas of mice express conserved, immunogenic 44-kilodalton oncofetal antigen. Am. J. Pathol 130:136.
8. Coggin, J. H., Jr., S. D. Rohrer, R. B. Hester, A. L. Barsoum, H. U. Rashid, and G. S. Gussack. 1993. 44-kd oncofetal transplantation antigen in rodent and human fetal cells. Implications of recrudescence in human and rodent cancers. Arch. Otolaryngol. Head Neck Surg. 119:1257.
9. Coggin, J. H., Jr. 1986. Oncofetal antigens. Nature (Lond) 319:428.
10. Barsoum, A. L., and J. H. Coggin, Jr. 1989. Immunogenicity of a soluble partially purified oncofetal antigen from murine fibrosarcoma in syngeneic mice. J. Biol. Resp. Mod. 8:579.
11. Rohrer, J. W., S. D. Rohrer, A. L. Barsoum, and J. H. Coggin, Jr. 1994. Differential recognition of murine tumor-associated oncofetal transplantation antigen and individually specific tumor transplantation antigens by syngeneic cloned BALB/c and RFM mouse T cells. J. Immunol. 152:754.
12. Rohrer, J. W., and J. H. Coggin, Jr. 1995. CD8 T cell clones inhibit antitumor T cell function by secreting IL-10. J. Immunol. 155:5719.
13. Rohrer, J. W., C. Culpepper, A. L. Barsoum, and J. H. Coggin, Jr. 1995. Characterization of RFM mouse T lymphocyte anti-oncofetal antigen immunity in apparent tumor-free, long-term survivors of sublethal X-irradiation by limiting dilution T lymphocyte cloning. J. Immunol. 154:2266.
14. Ménard, S., V. Castronovo, E. Tagliabue, and M. E. Sobel. 1997. New insights into the metastasis-associated 67 kD laminin receptor. J. Cell. Biochem 67:155.
15. Yow, H., J. M. Wong, H. S. Chen, C. Lee, G. Steele Jr., and L. B. Chen. 1988. Increased mRNA expression of a laminin-binding protein in human colon carcinomas: Complete sequence of a full-length cDNA encoding the protein. Proc. Natl. Acad. Sci. USA 85:6394.
16. Rao, C. N., V. Castronovo, M. C. Schmitt, U. M. Wewer, A. P. Claysmith, L. A. Liotta, and M. E. Sobel. 1989. Evidence for a precursor of the high-affinity metastasis-associated murine laminin receptor. Biochemistry 28:7476.
17. Wang, K. S., R. J. Kuhn, E. G. Strauss, S. Ou, and J. H. Strauss. 1992. High-affinity laminin receptor is a receptor for Sindbis Virus in mammalian cells. J. Virol. 66:4992.
18. Rabacchi, S. A., R. L. Neve, and U. C. Drager. 1990. A positional marker for the dorsal embryonic retina is homologous to the high-affinity laminin receptor. Development 109:521.
19. Makrides, S., S. T. Chitpatima, R. Bandyopadhyay, and G. Brawerman. 1988. Nucleotide sequence for a major messenger RNA for a 40 kilodalton polypeptide that is under translational control in mouse tumor cells. Nucleic Acids Res. 16:2349.
20. Auth, D., and G. Brawerman. 1992. A 33-kDa polypeptide with homology to the laminin receptor: Component of translation machinery. Proc. Natl. Acad. Sci. USA 89:4368.
21. Garcia-Hernandez, M., E. Davies, and P. R. Staswick. 1994. Arabidopsis p40 homologue, a novel acidic protein associated with the 40 S subunit of ribosomes. J. Biol. Chem. 269:20744.
22. Tohgo, A., S. Takasawa, H. Munkata, H. Yonekura, N. Hayashi, and H. Okamoto. 1994. Structural determination and characterization of a 40 kDa protein isolated from rat 40S ribosomal subunit. FEBS Lett. 340:133.
23. Rosenthal, E. T., and L. Wordeman. 1995. A protein similar to the 67 kDa laminin binding protein and p40 is probably a component of the translational machinery in Urechis caupo oocytes and embryos. J. Cell Sci. 108:245.
24. Westendorf, J. M., P. N. Rao, and L. J. Gerace. 1990. Mitotic phosphoprotein. J. Cell Biol. 111:93A.
25. Barsoum, A. L., and J. H. Coggin, Jr. 1993. Isolation and characterization of 200 kD oncofetal antigen from radiation induced murine lymphoma tumors. Int. J. Biochem. 25:483.
26. Barsoum, A. L., and J. H. Coggin, Jr. 1991. Isolation and partial characterization of a soluble oncofetal antigen from murine and human amniotic fluids. Int. J. Cancer 48:248.
27. Laemmli, U. K. 1970. Cleavage of structural proteins during the assembly of the head of bacteriophage T4. Nature 227:68.
28. Doolittle, R. F. 1977. Advanced Methods in Protein Sequence Determination. S. B. Needleman, ed. Springer, New York, p. 38.
29. Aebersold, R., J. Leavitt, L. E. Hood, and S. B. H. Kent. 1987. Sixth International Symposium on Methods in Protein Sequence Analysis. K. Walsh, ed. Humana, Clifton, N.J., p. 277.
30. Towbin, H., T. Staehlin, and J. Gordon. 1979. Electrophoretic transfer of proteins from polyacrylamide gels to nitrocellulose sheets: procedure and some applications. Proc. Natl. Acad. Sci. USA 76:4354.
31. Berger, M., T. A. Gaither, R. M. Cole, T. M. Chused, C. H. Hammer, and M. M.

Frank. 1982. Biotinylation of human C3. Molec. Immunol. 19:857.
32. Coggin, J. H., K. R. Ambrose, B. B. Bellomy, and N. G. Anderson. 1971. Tumor immunity in hamsters immunized with fetal tissues. J. Immunol. 107:526.
33. Ambrose, K. R., N. G. Anderson, and J. H. Coggin. 1971. Interruption of SV40 oncogenesis with human fetal antigens. Nature 233:194.
34. van den Brule, F. A., J. Price, M. E. Sobel, R. Lambotte, and V. Castronovo. 1994. Inverse expression of two laminin binding proteins, 67LR and galectin-3, correlates with the invasive phenotype of trophoblastic tissue. Biochem. Biophys. Res. Commun. 201:388.
35. Gussack, G. S., S. D. Rohrer, R. B. Hester, P. I. Liu, and J. H. Coggin, Jr. 1988. Human squamous cell carcinoma lines express oncofetal 44-kD polypeptide defined by monoclonal antibody to mouse fetus. Cancer 62:283.
36. Demeter, L. M., M. H. Stoler, M. E. Sobel, T. R. Broker, and L. T. Chow. 1992. Expression of high-affinity laminin receptor mRNA correlates with cell proliferation rather than invasion in human papillomavirus-associated cervical neoplasms. Cancer Res. 52:1561.
37. Sobel, M. E. 1993. Differential expression of the 67 kDa laminin receptor in cancer. Cancer Biol. 4:311.
38. Landowski, T. H., E. A. Dratz, and J. R. Starkey. 1995. Studies of the structure of the metastasis-associated 67 kDa laminin binding protein: Fatty acid acylation and evidence supporting dimerization of the 32 kDa gene product to form the mature protein. Biochemistry 34:11276.
39. Coggin, J. H. 1989. Shared cross-protective OFAs on chemically induced rodent sarcomas [Letter]. Immunol. Today 10:76.
40. Coggin, J. H. Jr. 1989. Cross-reacting tumor associated transplantation antigen on primary 3-methylcholanthrene-induced BALB/c sarcomas. Mol. Biother. 1:223.
41. Zocchi, M. R., M. Ferrarini, and C. Rugarli. 1990. Selective lysis of the autologous tumor by delta TCS1+ gamma/delta tumor infiltrating lymphocytes from human lung carcinomas. Eur. J. Immunol. 20:2685.
42. Ferrarini, M., S. M. Pupa, M. R. Zocchi, C. Rugarli, and S. Menard. 1994. Distinct pattern of HSP72 and monomeric laminin receptor expression in human lung cancers infiltrated by gamma/delta T lymphocytes. Int. J. Cancer 57:486.
43. Ferrarini, M., S. Heltai, S. M. Pupa, S. Menard, and M. R. Zocchi. 1996. Killing of laminin receptor-positive human lung cancers by tumor-infiltrating lymphocytes bearing gammadelta+ T-cell receptors. J. Natl. Cancer Inst. 88:436.
44. Cioce, V., I. M. Margulies, M. E. Sobel, and V. Castronovo. 1993. Interaction between the 67 kilodalton metastasis-associated laminin receptor and laminin. Kindney Int. 43:30.
45. Landowski, T. H., and S. Uthayakumar. 1995. Control pathways of the 67 kDa laminin binding protein: surface expression and activity of a new ligand binding domain. Clin. Exp. Metastasis 13:357.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

Applicants' parent application, U.S. application Ser. No. 08/835,069, filed Apr. 4, 1997, is also hereby incorporated by reference in its entirety.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A method for rendering T-suppressor cells cytotoxic, comprising administering to an individual who is a cancer patient an anti-IL-10 antibody.


SEQUENCE LISTING (1 sequence)

SEQ ID NO: 1 - protein, 294 aa, Unknown Organism
  Description of Unknown Organism Deduced amino acid sequence of 67LR cDNA
MSGALDVLQMKEEDVLKLLAAGTHLGGTNLDFQMEQYIYKRKSDGIYIINLKRTWEKLLLAARAIVAIENPADVSVISSRNTGQRAVLKFAAATGATPIAGRFTPGTPTNQIQAAFREPRLLVVTDPRADHQPLTEASYVNLPTIALCNTDSPLAYVDIAIPCNNRGAHSVGLMWWMLAREVLRMRGTISREHPWEVMPDLYFYRDPEETEKEEQAAABKVTKEEFQGEWTAPAPEFTAAQPEVADWSECVQVPSVPTQQFPTEDWSAQPATEDWSAAPTAQATEWVGATTEWS